(19) United States
(12) Patent Application Publication
(10) Pub. No.: US 2026/0242820 A1
(43) Pub. Date: Aug. 20, 2026

(54) AUTONOMOUS GENE CIRCUITS FOR THERAPY RELEASE

(71) Applicant: The Brigham and Women's Hospital, Inc., Boston, MA (US)

(72) Inventors: Shailesh Agarwal, Boston, MA (US); Vicki Ann Rosen, Chestnut Hill, MA (US)

(21) Appl. No.: 19/153,847

(22) PCT Filed: Feb. 9, 2024
(86) PCT No.: PCT/US2024/015271

§ 371 Date: Aug. 5, 2025

Related U.S. Application Data
(60) Provisional application No. 63/444,350, filed on Feb. 9, 2023.

(51) Int. Cl. C12N 15/85; C07K 14/52; C07K 14/71; C12N 5/071; C12N 5/077; C12N 5/0789; C12N 15/87

(52) U.S. Cl. CPC C12N 15/85; C07K 14/521; C07K 14/71; C12N 5/0647; C12N 5/0653; C12N 5/0669; C12N 5/0686; C12N 15/87; C07K 2319/30; C12N 2510/02; C12N 2510/04; C12N 2800/90; C12N 2830/34

(57) ABSTRACT

Current cell therapies and gene therapies used to increase or suppress the innate immune are often only temporarily effective due to clearance of reservoirs and the need to minimize risks of adverse systemic effects. Therefore, localized autonomous therapies are needed. Provided herein are transposon vectors and constructs for autonomously regulating expression and/or activity of proteins in inflammatory environments, and methods of their use.

### CROSS-REFERENCE TO RELATED APPLICATIONS

This patent application claims the benefit of, and priority to, U.S. Provisional Patent Application No. 63/444,350, filed Feb. 9, 2023, the content of which is incorporated herein by reference in its entirety.

### REFERENCE TO AN ELECTRONIC SEQUENCE LISTING

The contents of the electronic sequence listing (129319.01024.xml; Size: 61,040 bytes; and Date of Creation: Feb. 9, 2024) is herein incorporated by reference in its entirety.

### BACKGROUND OF THE INVENTION

Current cell therapies and gene therapies used to increase or suppress the innate immune are often only temporarily effective due to clearance of reservoirs and the need to minimize risks of adverse systemic effects. Therefore, localized autonomous therapies are needed.

### SUMMARY OF THE INVENTION

In an aspect, provided herein is a transposon vector comprising a construct, the construct comprising: a gene encoding a therapeutic protein operably linked to a heterologous promoter; wherein the therapeutic protein inhibits an activator protein; and wherein the activator protein activates transcription of the gene encoding the therapeutic protein.

In embodiments, the activator protein is an inflammatory protein; and the therapeutic protein is an anti-inflammatory protein. In embodiments, the promoter is selected from a C-C motif chemokine ligand 2 (CCL2) promoter, a bone morphogenetic protein responsive element (BRE) promoter, and a CAGA promoter.

In embodiments, the therapeutic protein is selected from C-C motif chemokine ligand 22 (CCL22), an activin receptor type IIA-Fc fusion protein (ActRIIA-Fc), a transforming growth factor beta receptor II-Fc fusion protein (TGFβRII-Fc), and a BMPRIA-Fc fusion protein.

In embodiments, the heterologous promoter is a C-C motif chemokine ligand 2 (CCL2) promoter; and the therapeutic protein is C-C motif chemokine ligand 22 (CCL22). In embodiments, the construct comprises a sequence having at least 95% identity to SEQ ID NO: 1.

In embodiments, the heterologous promoter is a bone morphogenetic protein responsive element (BRE) promoter; and the therapeutic protein is activin receptor type IIA-Fc fusion protein (ActRIIA-Fc). In embodiments, the construct comprises a sequence having at least 95% identity to SEQ ID NO: 3.

In embodiments, the heterologous promoter is a CAGA promoter; and the therapeutic protein is transforming growth factor beta receptor II-Fc fusion protein (TGFβRII-Fc). In embodiments, the construct comprises a sequence having at least 95% identity to SEQ ID NO: 5.

In embodiments, the heterologous promoter is a BRE promoter; and the therapeutic protein is a BMPRIA-Fc fusion protein. In embodiments, the construct comprises a sequence having at least 95% identity to SEQ ID NO: 13.

In another aspect, provided herein is a composition comprising any of the transposon vectors described herein; and a transposase enzyme.

In another aspect, provided herein is a kit comprising any of the transposon vectors described herein; and a transposase enzyme; wherein the transposon vector and transposase enzyme are provided in separate containers. In embodiments, the kit further comprises reagents for electroporation.

In another aspect, provided herein is a construct comprising a sequence having at least 95% identity to SEQ ID NO: 1, SEQ ID NO: 3, SEQ ID NO:5, or SEQ ID NO: 13.

In another aspect, provided herein is a host cell transfected with any of the transposon vectors or constructs described herein; wherein the host cell is capable of secreting the therapeutic protein. In embodiments, the host cell is a human cell or a mouse cell. In embodiments, the host cell is selected from an adipocyte, a bone marrow-derived cell, and a hematopoietic stem cell. In embodiments, the host cell is a primary cell. In embodiments, the host cell is an immortalized cell. In embodiments, the host cell is a human embryonic kidney cell.

### BRIEF DESCRIPTION OF THE DRAWINGS

FIG. 1 is a schematic depicting transgene delivery into purified adipocytes.

FIG. 2 illustrates a vector for CCL22 production upon exposure to TNFα using the CCL2 promoter.

FIGS. 3A-3B illustrate purification of adipocytes from human adipose tissue. (A) Processing of fat via mincing, homogenization, and filtration; (B) Purified adipocytes.

FIGS. 4A-4B illustrate transgene expression of (A) Bmpr1afc and (B) Tgfbr2fc by transfected adipocytes.

FIGS. 5A-5B illustrates that transfected adipocytes secrete (A) BMPR1AFc and (B) TGFβRII-Fc peptides.

FIG. 6 illustrates that TGFβRII-Fc and BMPR1A-Fc peptides secreted by transfected adipocytes are bioactive.

FIG. 7 TNFα-responsive CCL22 expression.

FIG. 8 illustrates that withdrawal of TNFα abolishes CCL22 production.

FIG. 9 illustrates secretion of CCL22 detected by ELISA.

FIGS. 10A-10B illustrate TREG migration assays with CCL22. (A) Transwell migration assay. (B) FACS analysis.

FIG. 11 illustrates a TREG migration assay.

FIG. 12 illustrates engraftment of electroporated tdTomato+ mouse adipocytes in the subcutaneous dorsum.

FIG. 13 is a coculture experiment showing mRNA expression of Bglap and Runx2.

FIG. 14 shows expression of BRE-ActR2AFc in mouse HSCs.

FIG. 15 shows expression of CAGA-TGFbR2-Fc in human adipocytes.

FIG. 16 shows in the number of TREGs in mice transplanted with adipocytes comprising CCL2-CCL22.

FIG. 17 illustrates the CCL2-CCL22 vector according to embodiments described herein.

FIG. 18 illustrates the BRE-ActRIIAFc vector according to embodiments described herein.

FIG. 19 illustrates the CAGA-TGFbRIIFc vector according to embodiments described herein.

FIG. 20 illustrates the BRE-BMPRIAFc transposon vector according to embodiments described herein.

### DETAILED DESCRIPTION

The present disclosure provides constructs, transposon vectors, and methods for expressing therapeutic proteins in the presence of a pathologic environment. Expression of a therapeutic protein is activated by a protein that exhibits high expression in the pathologic environment (i.e. an activator protein), and in turn suppresses expression of the activator protein, resulting in an autonomously regulated gene circuit. The constructs described herein may be provided in transposon systems for efficient and stable expression in a cell selected for therapeutic delivery.

### Transposon Vectors

In a first aspect, provided herein is a transposon vector comprising a construct, the construct comprising a gene encoding a therapeutic protein operably linked to a heterologous promoter; wherein the therapeutic protein inhibits an activator protein; and wherein the activator protein activates transcription of the gene encoding the therapeutic protein.

As used herein, the term “transposon” or “transposable element” refers to a polynucleotide that is able to excise from a donor polynucleotide, such as a vector, and integrate into a target site, such as genomic or extrachromosomal DNA of a cell. A transposon includes a polynucleotide sequence to be transposed (i.e., a construct comprising a heterologous promoter and a gene encoding a therapeutic protein) flanked by cis-acting nucleotide sequences on the termini of the transposon sequence. Cis-acting nucleotide sequences include at least one inverted repeat (also referred to as an inverted terminal repeat, or ITR), to which a transposase binds. As such, a “transposon vector” refers to a vector comprising a transposon capable of being transferred to a target site in genomic or extrachromosomal DNA.

As used herein, the term “transposase” refers to a polypeptide that catalyzes the excision of a transposon from a donor polynucleotide (e.g., a transposon vector) and the subsequent integration of the transposon into the genomic or extrachromosomal DNA of a target cell. Preferably, the transposase binds an inverted sequence or a direct repeat. The transposase may be provided to a cell as a polypeptide. Alternatively, the transposase may be provided as a polynucleotide that includes a coding sequence encoding the transposase. The polynucleotide can be RNA, such as an mRNA encoding the transposase, or a DNA sequence encoding the transposase. A transposase DNA coding sequence and the transposon may be on the same vector. Alternatively, the transposase coding sequence and the transposon may be on different vectors. In exemplary embodiments, the transposase is a mammalian piggyBac transposase (Di Matteo et al., 2012, Mobile Genetic Elements Volume 859). Other nonlimiting examples of transposition systems that may be included in the present compositions include Tn5, Tn3, Mu, Staphylococcus aureus Tn552, Tyl, Transposon Tn7, Tn/O and IS10, Tc1/mariner transposase (sleeping beauty), Tel, P Element, Tn3, bacterial insertion sequences, retroviruses, and retrotransposon of yeast (see, e.g., Mikkelsen, et al., 2003, In Vivo Mol Ther 8 (4), 654-665, Colegio et al., 2001, J. Bacteriol. 183:2384-8; Kirby et al., 2002, Mol. Microbiol. 43:173-86; Devine and Boeke, 1994, Nucleic Acids Res., 22:3765-72; International Patent Application No. WO 95/23875; Craig, 1996, Science 271:1512; Craig, 1996, Review in: Curr Top Microbiol Immunol. 204:27-48; Kleckner et al., 1996, Curr Top Microbiol Immunol. 204:49-82; Lampe et al., 1996, EMBO J. 15:5470-9; Plasterk, 1996, Curr Top Microbiol Immunol 204:125-43; Gloor, 2004, Methods Mol. Biol. 260:97-114; Ichikawa and Ohtsubo, 1990, J Biol. Chem. 265:18829-32; Ohtsubo and Sekine, 1996, Curr. Top. Microbiol. Immunol. 204:1-26; Brown et al., 1989, Proc Natl Acad Sci USA 86:2525-9; and Boeke and Corces, 1989, Annu Rev Microbiol. 43:403-34).

As used herein, the term “nucleic acid” or “polynucleotide” refers to deoxyribonucleic acid (DNA), ribonucleic acid (RNA) and DNA/RNA hybrids. Polynucleotides may be single-stranded or double-stranded. Nucleic acids include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA (+)), minus strand RNA (RNA (−)), synthetic RNA, genomic DNA (gDNA), PCR amplified DNA, complementary DNA (cDNA), synthetic DNA, or recombinant DNA.

As used herein, the term “construct” refers to a recombinant polynucleotide, i.e., a polynucleotide that was formed artificially by combining at least two polynucleotide components from different sources (natural or synthetic). For example, the constructs described herein comprise the coding region of a gene encoding a therapeutic protein operably linked to a promoter. Constructs can be generated using conventional recombinant DNA methods. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

As used herein, the term “vector” refers to a nucleic acid molecule capable of propagating another nucleic acid to which it is linked. The term includes the vector as a self-replicating nucleic acid structure as well as a vector incorporated into the genome of a host cell into which it has been introduced. The term vector encompasses “plasmids”, the most commonly used form of vector. Plasmids are circular double-stranded DNA loops into which additional DNA segments (e.g., those encoding peptides) may be ligated. In some embodiments, the vector is a mini-circle DNA (mcDNA) vector. Mini-circle DNA vectors are episomal DNA vectors that are produced as circular expression cassettes devoid of any bacterial plasmid DNA backbone. See, for example, System Biosciences, Mountain View CA, MN501A-1. Their smaller molecular size enables more efficient transfections and offers sustained expression over a period of weeks as compared to standard plasmid vectors that only work for a few days. The vectors may further a comprise heterologous nucleic acid backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of encoded peptides.

The constructs described herein comprise a gene encoding a therapeutic protein operably linked to a promoter that increases transcription of the therapeutic protein when the construct is in the presence of an activator protein.

As used herein, the term “therapeutic protein” refers to a protein that, when present in an effective amount, produces a desired therapeutic and/or physiologic effect on a subject in need thereof. It includes a protein that has a measurable or conveyable effect on the subject, e.g. it alleviates or decreases a symptom of a disease, disorder, or condition. The desired physiologic effect may include reducing or modulating a pathologic condition in the local environment in which the protein is expressed. In embodiments, the therapeutic protein is an anti-inflammatory protein. As used herein, the term “anti-inflammatory protein” refers to a protein or peptide that promotes an immunosuppressive environment, such as by activating expression or activity of proteins that confer immunosuppression or by downregulating expression or activity of proteins that confer inflammation.

As used herein, the term “activator protein” refers to a protein that is characteristically expressed in a pathologic environment in the absence of the therapeutic protein or when the therapeutic protein is expressed at normal or low levels. For example, the activator protein may exhibit increased expression, or it may be solely expressed during a pathologic condition or disease state. The activator protein may be a native or endogenous protein to the subject in which it is expressed. Alternatively, the activator protein may be an infectious agent, such as a protein expressed by a bacteria or virus. In some embodiments, the activator protein causes or aggravates the pathologic environment. When cells comprising the construct described herein are introduced to the pathologic environment, the activator protein directly or indirectly induces expression of the therapeutic protein, which in turn modulates expression or activity of the activator protein and alleviates the pathologic or disease condition. In embodiments, the activator protein is an inflammatory protein. As used herein, the term “inflammatory protein” refers to a protein or peptide that promotes an inflammatory environment, such as by activating expression or activity of proteins that confer inflammation or by downregulating expression or activity of proteins that confer immunosuppression.

As used herein, the terms “protein” or “polypeptide” or “peptide” are used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

The proteins disclosed herein include wild type proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

In an embodiment, the therapeutic protein is C-C motif chemokine ligand 22 (CCL22), and the heterologous promoter is a C-C motif chemokine ligand 2 (CCL2) promoter (CCL2-CCL22 construct). CCL2 is a small pro-inflammatory cytokine of the CC chemokine family that recruits monocytes, memory T cells, and dendritic cells to sites of inflammation produced by tissue injury or infection. The CCL2 promoter is activated in the presence of activator protein, TNFα. In contrast, CCL22 is an immunosuppressant, and plays a role in the recruitment of regulatory T cells (Tregs) to sites of tissue injury. CCL22 is of interest as a therapeutic target for preventing rejection and deleterious side effects of allotransplantation. The inventors have placed CCL22 under the CCL2 promoter so that it may be upregulated in the presence of an inflammatory environment and high expression of TNFα. When the promoter is activated by TNFα, increased expression of CCL22 promotes an immunosuppressive environment, which leads to reduced expression and/or activity of TNFα, resulting in an autonomously controlled gene circuit.

A transposon vector comprising the CCL2-CCL22 construct may comprise SEQ ID NO: 2, or a sequence having at least 80%, 85%, 90%, 91%, 92%, 93%, 94%, 95%, 96%, 97%, 98%, or 99% identity thereto.

In an embodiment, the therapeutic protein is activin receptor type IIA-Fc fusion protein (ActRIIA-Fc), and the heterologous promoter is bone morphogenic protein responsive element (BRE) (BRE-ActRIIAFc construct). The BRE promoter is activated in the presence of Activin A. As described in the Examples, patients with fibrodysplasia ossificans progressive (FOP) have a mutation in the Activin A receptor type I (ACVR1) gene (R206H mutation). This mutation confers ACVR1 signaling hyperactivity in the presence of Activin A, which has been demonstrated to play a role in FOP lesion burden. ActRIIA-Fc serves as a ligand trap for Activin A and reduces its activity. In patients with FOP, binding of Activin A to the mutated ACVR1 (ACVR1 R206H) leads to up-regulation of BMP signaling. BMP receptor activation leads to phosphorylation of Smad 1/5 complex; phospho Smad1/5 translocates into the nucleus to transactivate promoters including BRE. Therefore, when Activin A is highly expressed, activation of the BRE promoter increases transcription of ActRIIA-Fc, which in turn inhibits Activin A activity, resulting in an autonomously controlled gene circuit.

A transposon vector comprising the BRE-ActRIIAFc construct may comprise SEQ ID NO: 4, or a sequence having at least 80%, 85%, 90%, 91%, 92%, 93%, 94%, 95%, 96%, 97%, 98%, or 99% identity thereto.

In another embodiment, the therapeutic protein is TGFβRII-Fc, and the heterologous promoter comprises a CAGA box (CAGA-TGFβRIIFc construct). A promoter that comprises a CAGA box may be referred to herein as a CAGA promoter. TGβRII-Fc is a ligand trap TGβ1/3 ligands. TGFβ activates the CAGA promoter; in particular TGFβ ligands bind to TGFβ receptor which phosphorylates Smad 2/3 through its kinase domain. Phospho-Smad 2/3 can then translocate into the nucleus and transactivate CAGA promoter, leading to gene expression.

A transposon vector comprising the CAGA-TGFβRIIFc construct may comprise SEQ ID NO: 6, or a sequence having at least 80%, 85%, 90%, 91%, 92%, 93%, 94%, 95%, 96%, 97%, 98%, or 99% identity thereto.

In another embodiment, the therapeutic protein is a bone morphogenetic protein receptor type IA (BMPR1A)-Fc fusion protein (BMPR1A-Fc), and the heterologous promoter is BRE (BRE-BMPR1A-Fc construct). This construct may be used to treat pathologies in which excessive BMP ligand is present, such as FOP and post-traumatic heterotopic ossification (in which the BMP receptor is not mutated). in this setting, BMPR1A-Fc is a ligand trap for BMP ligand.

A transposon vector comprising the BRE-BMPR1A-Fc construct may comprise SEQ ID NO: 14, or a sequence having at least 80%, 85%, 90%, 91%, 92%, 93%, 94%, 95%, 96%, 97%, 98%, or 99% identity thereto.

In any of the embodiments, the transposon vector may comprise solely the components necessary to transpose the construct into genomic or extrachromosomal DNA.

In any of the vectors and constructs described herein, the heterologous promoter may be a promoter that is activated by an inflammatory environment, and the therapeutic protein may be a protein that is able to suppress a protein that induces or aggravates the inflammatory environment.

In some embodiments, the heterologous promoter is selected from a CCL2 promoter (SEQ ID NO: 7), a BRE promoter (SEQ ID NO: 9), and a CAGA promoter (SEQ ID NO: 11). The heterologous promoter may be a promoter that is activated by TGFβ, Activin A, BMP, or any other inflammatory ligand.

In some embodiments, the therapeutic protein is selected from CCL22 (SEQ ID NO: 8), ActRIIA-Fc (SEQ ID NO: 10), TGFβRII-Fc (SEQ ID NO: 12), and BMPR1A-Fc (SEQ ID NO: 15). The therapeutic protein may be a protein that has anti-inflammatory capacity.

### Constructs

In a second aspect, provided herein are constructs useful in disease modulating gene circuits.

The CCL2-CCL22 construct described herein may comprise SEQ ID NO: 1 or a sequence having at least 80%, 85%, 90%, 91%, 92%, 93%, 94%, 95%, 96%, 97%, 98%, or 99% identity thereto.

The BRE-ActRIIAFc construct described herein may comprise SEQ ID NO: 3 or a sequence having at least 80%, 85%, 90%, 91%, 92%, 93%, 94%, 95%, 96%, 97%, 98%, or 99% identity thereto.

The CAGA-TGFβRIIFc construct described herein may comprise SEQ ID NO: 5, or a sequence having at least 80%, 85%, 90%, 91%, 92%, 93%, 94%, 95%, 96%, 97%, 98%, or 99% identity thereto.

The BRE-BMPR1A-Fc construct described herein may comprise SEQ ID NO: 13, or a sequence having at least 80%, 85%, 90%, 91%, 92%, 93%, 94%, 95%, 96%, 97%, 98%, or 99% identity thereto.

The term “substantial identity” of polynucleotide sequences means that a polynucleotide comprises a sequence that has at least 85% sequence identity to the SEQ ID. Alternatively, percent identity can be any integer from 85% to 100%. More preferred embodiments include at least: 85%, 86%, 87%, 88%, 89%, 90%, 91%, 92%, 93%, 94%, 95%, 96%, 97%, 98% or 99% compared to a reference sequence using a sequence alignment program; preferably BLAST using standard parameters. These values can be appropriately adjusted to determine corresponding identity of proteins encoded by two nucleotide sequences by taking into account codon degeneracy, amino acid similarity, reading frame positioning, and the like.

“Substantial identity” of amino acid sequences for purposes of this invention normally means polypeptide sequence identity of at least 85%. Preferred percent identity of polypeptides can be any integer from 85% to 100%. More preferred embodiments include at least 85%, 86%, 87%, 88%, 89%, 90%, 91%, 92%, 93%, 94%, 95%, 96%, 97%, 98%, or 99% to a reference sequence using a sequence alignment program; preferably BLAST using standard parameters.

### Compositions

In a third aspect, provided herein are compositions comprising any of the transposon vectors comprising the constructs described herein and corresponding transposase enzymes.

### Kits

In a fourth aspect, provided herein are compositions comprising any of the transposon vectors comprising the constructs described herein and corresponding transposase enzymes, wherein the vectors and enzymes are packaged in separate containers. The transposon vectors and transposase enzymes may be lyophilized. The kit may further include buffers and reagents necessary for transfecting a cell with the transposon vector and enzyme. The reagents may comprise any reagents necessary for transfecting the cells by electroporation. The kit may further include a dropper for dispensing controlled volumes of liquid, such as a controlled volume disposable Pasteur pipette.

### Host Cells

In a fifth aspect, provided herein is a host cell transfected with any of the transposon vectors or any of the constructs described herein, wherein the cell is capable of secreting the therapeutic protein. The host cell must be capable of responding to the activator protein in the local environment by activating transcription at the heterologous promoter. As used herein, the term “host cell” refers to any prokaryotic or eukaryotic cell that contains a vector or construct described herein. This term also includes cells that have been genetically engineered such that a construct described herein is integrated into its genome. Preferably, the host cell is a eukaryotic cell. More preferably, the host cell is a mammalian cell, including but not limited to, a mouse cell and a human cell. In preferred embodiments, the host cell is a human cell.

The host cell may be a primary cell or a cell of an immortalized cell line.

In exemplary embodiments, host cells include adipocytes, bone marrow-derived cell, hematopoietic stem cells, and human embryonic kidney cells (e.g HEK293).

The terms “transduced,” “transfected,” and “transformed” refer to processes by which an exogenous nucleic acid is introduced into a host cell. The vectors described herein may be used to transfect the construct into a host cell.

### Methods

In a seventh aspect, provided herein is a method of inducing local immunosuppression at a site of inflammation in a subject in need thereof, the method comprising transfecting a host cell with a transposon vector described herein, and injecting the cell at the site of local immunosuppression.

The site of inflammation may be a site of allotransplantation, a site of injury, a tumor site, etc.

In an embodiment, the method comprises extracting adipose tissue from the subject; dissociating the adipose tissue into individual cells; isolating adipocytes from the individual cells; transfecting the adipocytes with a transposon vector described herein; and introducing the transfected adipocytes into the subject at the site of inflammation. Introducing the construct at the site of allotransplantation promotes local immunosuppression at the site, thereby avoiding the effects of systemic immunosuppression that occurs with the current methods known in the art.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like. In preferred embodiments, the subject is a human.

In this aspect, the subject has undergone or will undergo a cell, tissue, or organ transplant or engraftment.

“Adipose tissue”, which is also referred to as “fat” or “body fat”, is a loose connective tissue composed of adipocytes, preadipocytes, fibroblasts, vascular endothelial cells, and a variety of immune cells (e.g., adipose tissue macrophages).

The method may be performed as described in PCT Publication No. WO2022060810A1, which is incorporated herein by reference in its entirety. Adipose tissue may be harvested in a semi-solid state from the subject using liposuction or may be harvested as whole adipose tissue using surgical techniques. In preferred embodiments, the adipose tissue is extracted from the subject by liposuction. Dissociation produces a cell suspension that is amenable to electroporation, unlike whole adipose tissue. Tissue dissociation can be accomplished using enzymatic dissociation, chemical dissociation or mechanical dissociation. In preferred embodiments, the tissue is dissociated by mechanical dissociation. The minced-up tissue produced by mechanical dissociation may then be washed to separate the cells from the tissue. Gentle agitation may also be used to help loosen the cells. Adipocytes may be isolated from the tissue by filtering the cells through a strainer, centrifuging the filtrate at 50 g for 3 minutes to separate the mixture into a top layer comprising free oil, a middle layer comprising dissociated adipocytes, and a bottom layer comprising buffer, and removing the top layer and bottom layer to isolate the middle layer comprising the adipocytes. Thus, in some embodiments, isolating adipocytes comprises (i) filtering the individual cells generated in step (b) to obtain a filtrate; (ii) centrifuging the filtrate; and (iii) harvesting the adipocytes from the centrifuged filtrate.

In preferred embodiments, the isolated adipocytes are transfected with the transposon vector or construct by electroporation. “Electroporation” is a physical transfection method that uses an electrical pulse to create temporary pores in cell membranes through which substances like nucleic acids can pass into cells. In the examples, experiments are described in which a variety of electroporation settings for transfection of adipocytes are tested, e.g., 100-1000V, 1-10 pulses, and 1-10 msec. Based on these experiments, it has been determined that the optimal electroporation settings for gene delivery and adipocyte survival are 500V, 4 pulses, and 5 msec per pulse. Thus, in some embodiments, electroporation is performed using those optimized electroporation settings.

In preferred embodiments, transfected adipocytes re introduced into the subject in a manner that allows for local delivery, e.g. by injection into the site of transplantation. The method may be performed before, after, or during the transplantation procedure.

The adipose tissue or adipocytes may be extracted from the subject being treated, or another subject. In preferred embodiments, the adipose tissue or adipocytes are extracted from a human.

In an exemplary embodiment, the site of inflammation is a site of allotransplantation, and the construct is a CCL2-CCL22 construct.

In another embodiment, provided herein is a method of inducing local immunosuppression at a site of injury in a subject in need thereof, the method comprising extracting whole bone marrow or hematopoietic stem cells from the subject; transfecting the cells with a transposon vector described herein; and introducing the transfected cells into the subject at the site of injury. Introducing the construct at the site of injury promotes local immunosuppression at the site, thereby avoiding the effects of systemic immunosuppression that occurs with the current methods known in the art.

In this aspect, the subject has been diagnosed with fibrodysplasia ossificans progressive (FOP). The method may be performed before, during, or after an injury has occurred. As described herein, the term “injury” refers to a site of an abrasion, bruise, wound, fracture, or other sites of impact that break the skin or other body tissues.

In embodiments, the cells may be extracted and transfected, then stored for a period of time under conditions that maintain viability of the cells before being reintroduced to the subject. In preferred embodiments, the cells are transfected by electroporation.

In an exemplary embodiment, the site of inflammation is an injury in a subject having FOP, and the construct is a BRE-ActRIIAFc construct.

In other embodiments, the site of inflammation is a tumor in a subject, wherein the tumor has fibrotic surroundings. For example, the tumor may be a melanoma, a breast cancer tumor, a pancreatic tumor, etc. The cells may be introduced into the tumor or into the surrounding tissue.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

As used herein, the term “introducing” and “administering” refer to dispensing, delivering or applying the substance to the subject. In terms of the cellular compositions described herein, “introducing” is intended to refer to delivering them to the subject by any suitable route for delivery of the therapeutic agent to the desired location within the subject. As discussed above, suitable modes of administration include, without limitation: intralesional, intraorgan, intratumoral, etc.

As used herein “control,” as in “control subject” or “control sample” has its ordinary meaning in the art, and refers to a sample, or a subject, that is appropriately matched to the test subject or test sample and is treated or not treated as appropriate.

### Miscellaneous

Unless otherwise specified or indicated by context, the terms “a”, “an”, and “the” mean “one or more.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are illustrative and should not be interpreted to limit the scope of the claimed subject matter.

### Example 1

Allotransplantation presents an enormous clinical reconstructive opportunity for individuals who have experienced significant musculoskeletal trauma or burn injuries. However, the adoption of this transformative surgical option has been limited, in large part, due to the risks and costs associated with traditional lifelong immunosuppression strategies including corticosteroids and calcineurin inhibitors. These risks are substantial and include metabolic disorders leading to diabetes, hyperlipidemia, poor wound healing, and a predisposition to developing post-transplant malignancies. Therefore, there is a critical need for strategies which enable local immunosuppression while maintaining immunocompetence across the recipient outside of the transplanted tissue. Here, the inventors build on a therapeutic strategy in which autologous adipocytes are modified to produce and secrete an immunomodulatory chemokine (C-C motif chemokine ligand 22 or CCL22), which augments local recruitment of immunosuppressive regulatory T-cells (Tregs), using a closed-loop regulatory mechanism.

Activated T-cells are recognized as the predominant cellular mediator of acute rejection in the context of solid organ and composite allotransplantation. These alloreactive T-cells are primed for alloantigen recognition via interaction with antigen presenting cells (APCs) often in the draining lymph nodes. Consistent with this role for activated T-cells, lymphocyte depletion agents such as alemtuzumab and antithymocyte globulin have had a longstanding role in post-transplantation immunosuppressive regimens.

Regulatory T-cells (Tregs) have received attention as a potential therapy to prevent allograft rejection due to their immunosuppressive function. The promise of Tregs in solid organ transplantation is under intense investigation through a series of clinical trials involving Treg infusion⁹. This function is believed to be mediated in part through binding of CD80/86 on Tregs with immunosuppressive CTLA-4 on T-cells and APCs. Indeed, antibody-mediated repression of CTLA-4 (e.g. ipilimumab) blocks Tregs, and is used as part of immunotherapy against tumors.

C-C motif chemokine ligand 22 (CCL22) recruits Tregs to sites of tissue injury. In the setting of autoimmune diabetes, CCL22-mediated Treg recruitment conferred protection against T-cell mediated pancreatic islet cell injury; this effect was verified by the return of autoimmune injury upon depletion of Tregs using anti-CD25 antibody. Others have shown that intra-tumoral CCL22 reduces the efficacy of anti-tumor immunotherapy, presumably through the CD80/86-CTLA4 immunosuppressive axis. The promising therapeutic opportunity offered by CCL22 in transplantation is supported by recent studies demonstrating improved graft survival in murine models. Pancreatic islet cell transplants transfected with adenovirus expressing Ccl22 enjoyed improved graft survival through 80 days. Artzi et al. have shown that local CCL22 injection augments Treg recruitment to skin allograft and reduces tissue rejection. In a rat model of hindlimb VCA, Little et al. showed that local delivery of microparticle-encapsulated CCL22 enabled long-term graft survival. Together, these findings suggest that CCL22 delivery presents a novel therapeutic strategy which deserves further investigation in allotransplantation.

Despite the local immunosuppression offered by CCL22, the prospect of permanent graft survival remains elusive due to limitations of current CCL22 delivery systems. Microparticles or scaffolds offer only transient CCL22 delivery until the reservoir is depleted; in addition these strategies may not be amenable to precisely-regulated delivery. Similarly, adenoviral gene therapy offers only transient delivery; while lentiviral delivery allows for stable genomic integration, the risks of systemic immunologic response are particularly concerning in patients already at risk for rejection due to a heightened immune response. A strategy which enables local, long-term production and delivery would obviate concerns regarding treatment durability.

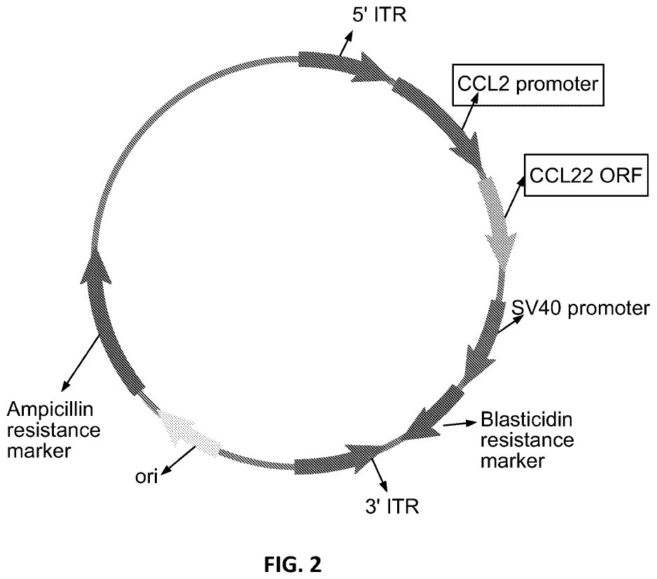

Although local production of an immunosuppressive cytokine presents a promising strategy for local immunosuppression, constitutive long-term production presents concerns regarding systemic immunosuppression. A closed-loop gene circuit to control CCL22 production would further enable regulated delivery precisely “tuned” to the graft requirements. One strategy to enable closed-loop, autonomous CCL22 production is based on activated expression in the presence of inflammatory cytokines. Tumor necrosis factor α (TNFα) is one such cytokine which is present during acute rejection, providing a potential stimulus for CCL22 production given the appropriate promoter. The promoter for C-C motif chemokine ligand 2 (CCL2) is one such sequence which is activated in the presence of TNFα and has been previously incorporated in engineered closed-loop cells. To that end, the inventors have developed a cell therapy composed of autologous adipocytes (“fat cells”) which are mechanically isolated and modified to express and secrete bioactive CCL22 using the TNFα-responsive CCL2 promoter (FIG. 2).

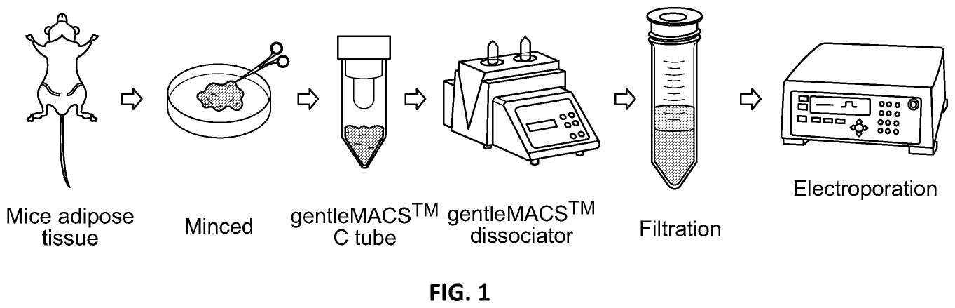

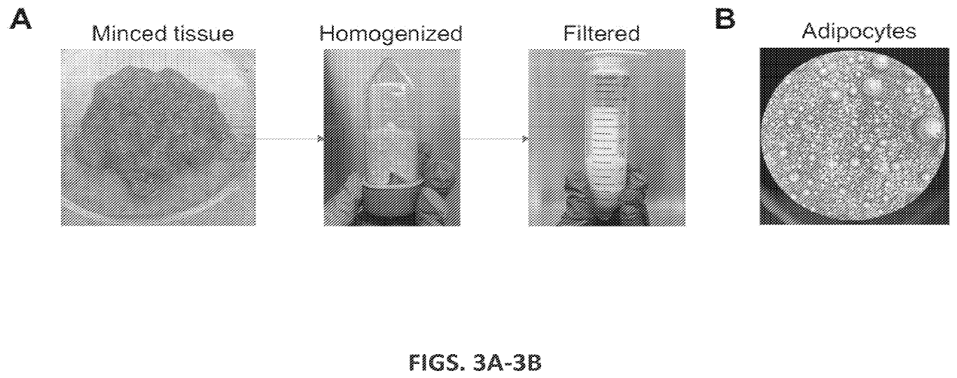

To mechanically isolate adipocytes from mouse or human adipose tissues, first the adipose tissue obtained via excision is morselized and homogenized mechanically. Then, homogenate is filtered (100-um filter), and the filtrate is centrifuged (50 g×3 mins); and floating adipocytes are harvested (FIGS. 1 and 3A). Purified adipocytes have been consistently obtained using this approach (FIG. 3B).

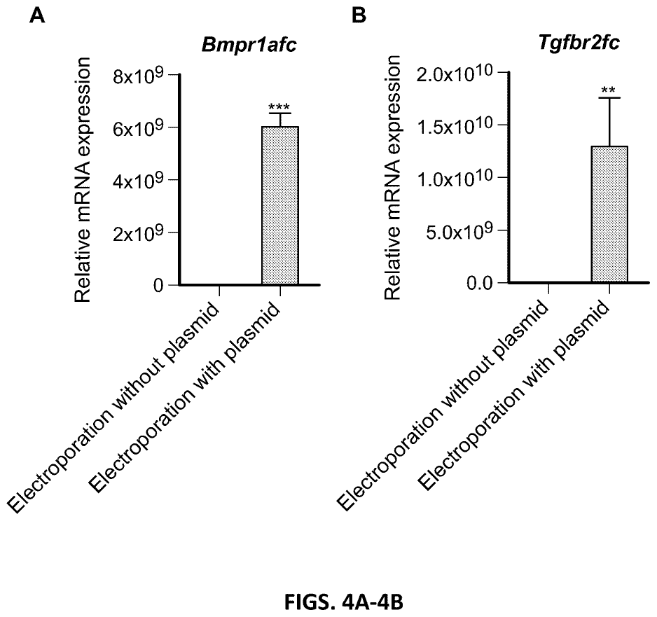

The inventors have developed a strategy for transgene delivery into purified adipocytes using electroporation (FIG. 1). Adipocytes obtained as above from adipose tissue were electroporated with square waves (500 V/5 msec/4 pulses) to deliver 20 μg of plasmid DNA (CMV-Bmpr1afc, CMV-Tgfbr2fc) encoding BMPR1A-Fc, a decoy receptor which binds and inhibits bone morphogenetic protein 2 (BMP2) activity²¹, or TGFβRII-Fc, a decoy receptor which binds and inhibits transforming growth factor β 1/3 (TGFβ1/3) activity. After 7 days in culture, qPCR demonstrated expression of the desired transgene in adipocytes transfected with CMV-driven plasmids when compared with negative controls (adipocytes electroporated without plasmid) (FIGS. 4A and 4B) (n>3 replicates).

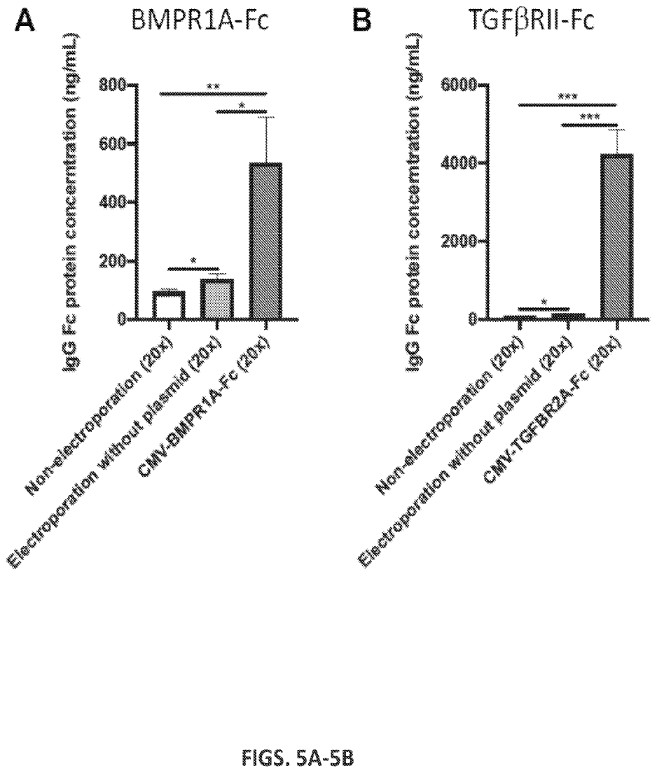

Upon demonstration of transgene expression, the inventors then sought to demonstrate that these adipocytes can secrete bioactive peptides. ELISA of 20×-concentrated media demonstrated secretion of the desired BMPR1A-Fc and TGFβRII-Fc peptides by adipocytes when compared with negative controls (n>3 replicates) (FIGS. 5A and 5B).

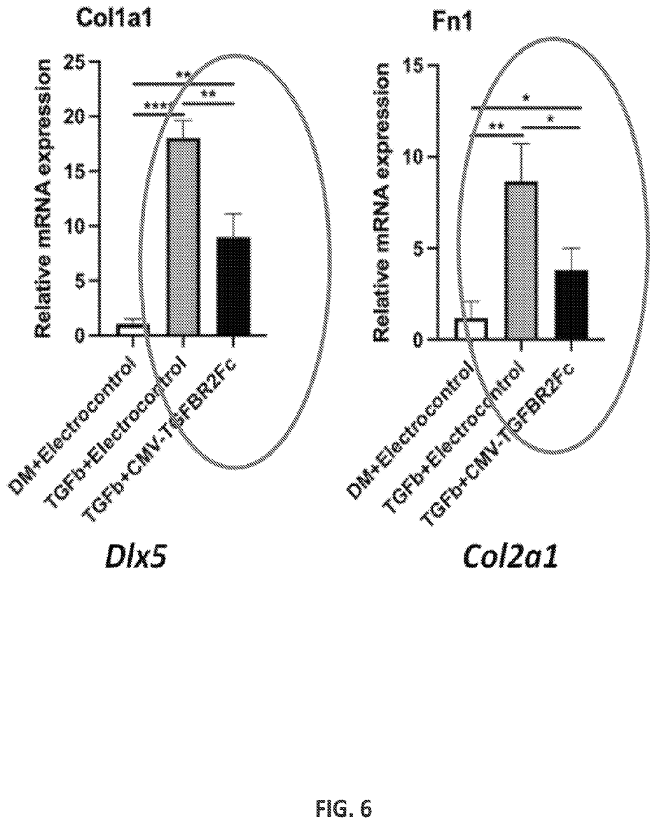

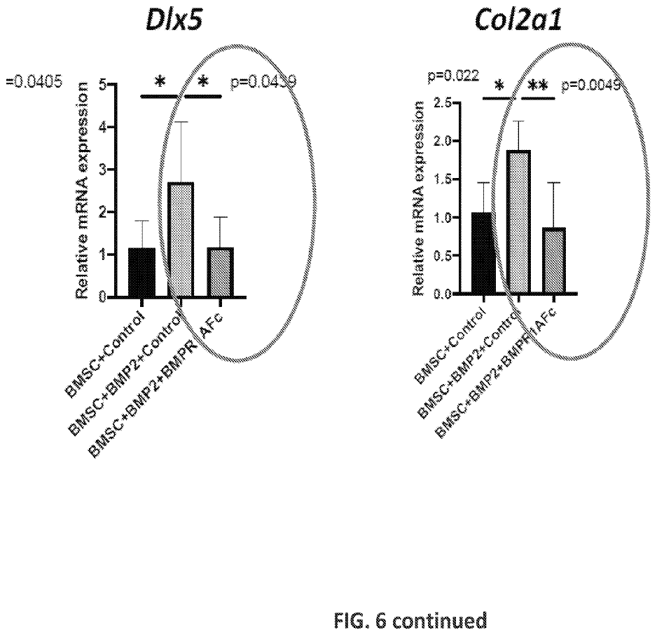

Co-culture experiments confirmed bioactivity of the desired peptides. In particular, upon co-culture of TGFβ1-treated bone marrow-derived mesenchymal cells (BM-MSCs) with adipocytes modified to secrete TGFβRII-Fc (inhibitor of TGFβ1/3), the inventors found reduced BM-MSC expression of fibrogenic Colla1 and Fn1 (FIG. 6) (n>3 replicates). Similarly, upon co-culture of BMP2-treated bone marrow-derived mesenchymal cells (BM-MSCs) with adipocytes modified to secrete BMPR1A-Fc (inhibitor of BMP2), the inventors found reduced BM-MSC expression of osteogenic Dlx5 and Col2a1 (FIG. 6) (n>3 replicates).

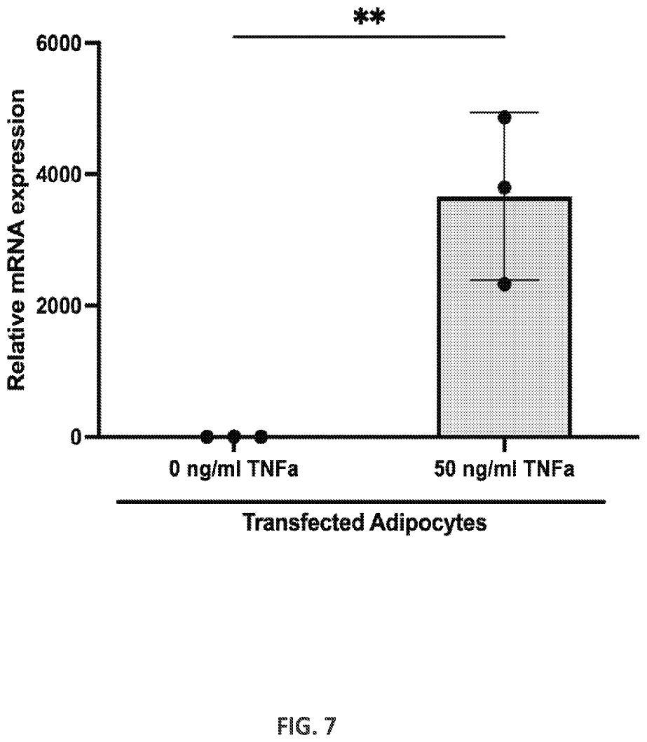

The success with these recombinant peptides led the inventors to develop a piggyBAC transposon mini-plasmid encoding CCL22 transgene expression. They further modified this plasmid to enable CCL22 expression driven by the CCL2 promoter (Promoter (CCL2)-CCL22). Adipocytes were electroporated (500 V/5 msec/4 pulses) with the mini-plasmid or no plasmid and then cultured in vitro in DMEM/10% FBS/1% pen-strep+/−TNFα (50 ng/ml). After 7 days in culture, a significant increase in CCL22 transgene expression was observed in adipocytes transfected with Promoter (CCL2)-CCL22 plasmid and treated with TNFα, when compared with adipocytes transfected with Promoter (CCL2)-CCL22 plasmid without TNFα treatment (n=3 replicate wells; 3 technical triplicates) (FIG. 7).

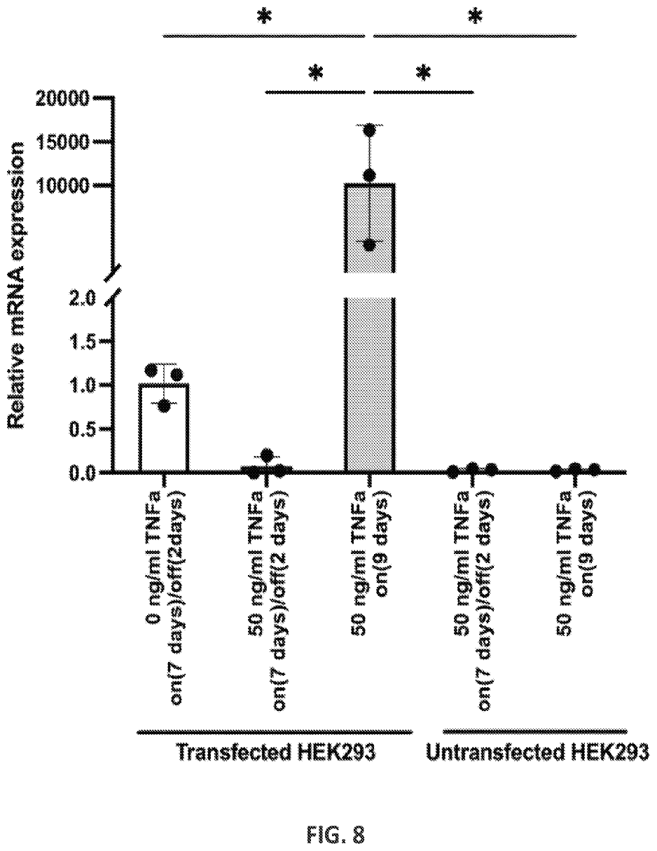

Following this, the inventors sought to demonstrate whether withdrawal of TNFα could lead to a compensatory reduction in CCL22 expression, indicative of a closed-loop system. HEK293 cells transfected with piggyBAC mini-plasmids encoding promoter (CCL2)-CCL22 and treated with TNFα (50 ng/mL) for 9 days exhibited persistent CCL22 gene expression, while those treated with TNFα (50 ng/ml) for 7 days followed by 2 days of TNFα withdrawal exhibited abolished CCL22 expression (FIG. 8).

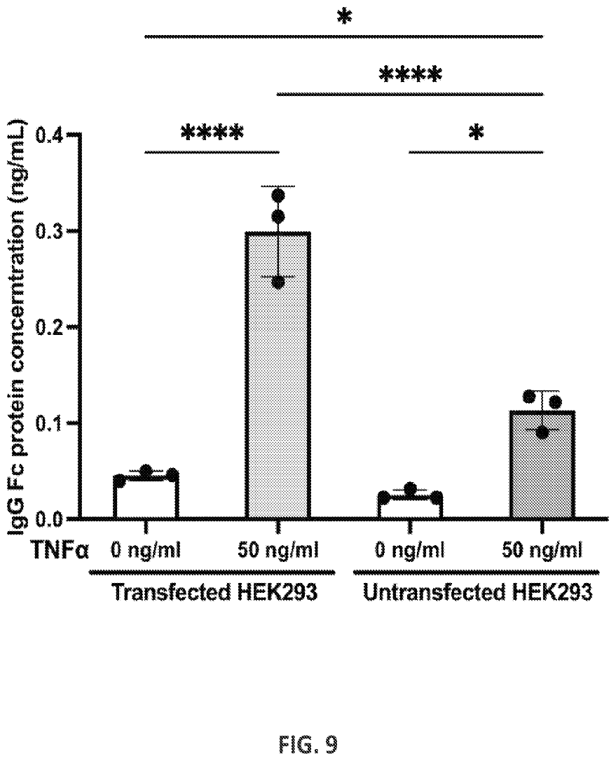

The inventors then sought to demonstrate secreted CCL22 is bioactive. ELISA (anti-CCL22 kit (Abcam)) of 20×-concentrated media demonstrated secretion of the desired CCL22 peptide by HEK293 when compared with negative controls (n>3 replicates) (FIG. 9).

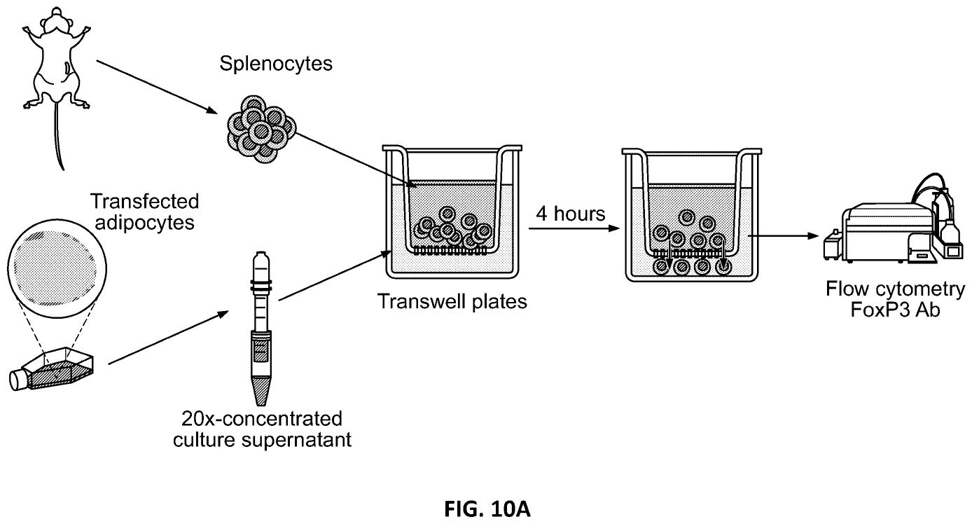

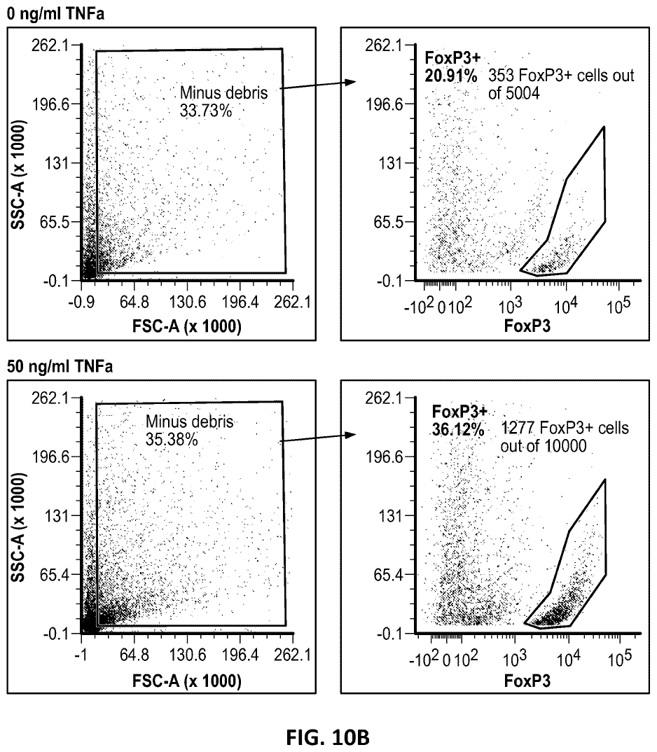

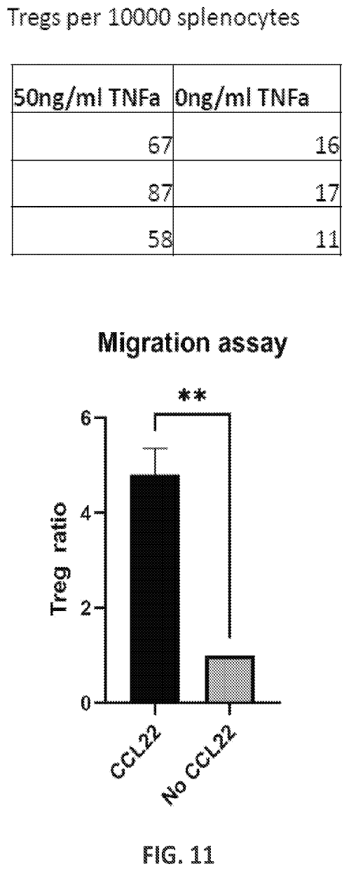

Next, to demonstrate CCL22 bioactivity, the inventors performed transwell migration assays (FIG. 10A). Mouse splenocytes were cultured in vitro and 10⁶ cells were loaded onto the upper chamber of Transwell plates (Corning) in DMEM/10% FBS/1% pen-strep. The bottom chamber was loaded with either 1) 20×-concentrated culture media from 1) transfected (Promoter (CCL2)-CCL22) HEK293 treated with 50 ng/ml TNFα OR 2) transfected (PromoterCCL2)-CCL22) HEK293 cells treated with 0 ng/ml TNFα. In the latter, TNFα was added into the 20×-concentrated media to control for the effect of residual TNFα on splenocyte differentiation. The inventors found increased presence of FOXP3+ Tregs in the bottom chamber within 12 hours upon treatment with cultured media from TNFα-treated HEK293 cells based on FACS (36% v. 21%) (FIGS. 10B and 11).

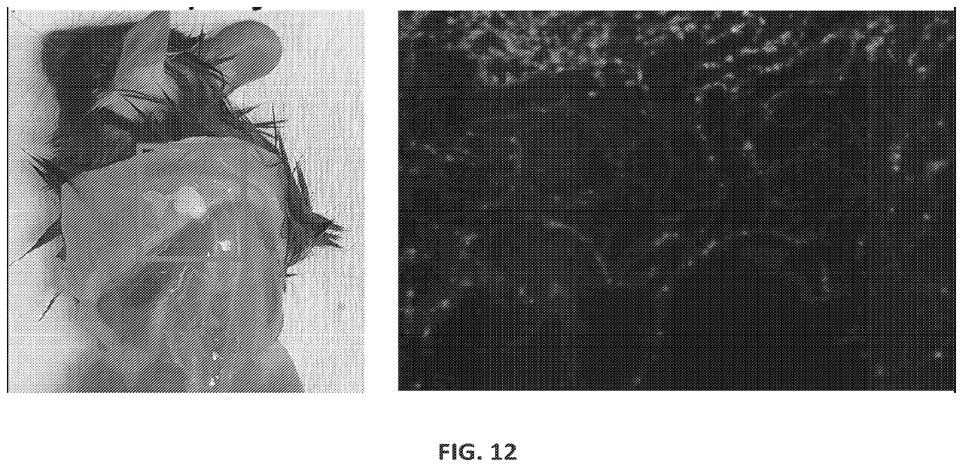

Next, the inventors showed that electroporated adipocytes are able to engraft into the subcutaneous dorsum, the planned recipient site for both the skin allograft and the isogenic adipocytes. Adipocytes were isolated from C57BL/6 mice with red fluorescent protein and subjected to the electroporation settings required for transfection (500V/5 msec/4 pulses). Electroporated adipocytes were injected into the subcutaneous dorsum of unlabeled wild type C57BL/6 mice; recipient mice were euthanized after 2 weeks and the dorsum evaluated for gross and histologic presence of injected adipocytes. There was robust adipocyte engraftment on the basis of discernible subcutaneous adipose tissue located at the site of injection with the desired RFP+ signal on histologic evaluation (FIG. 12).

The inventors tested the system in vivo using the following procedure:
1. Transfected adipocytes with PromoterCCL2-CCL22 construct, or null plasmid
2. Treated with TNFα in vitro×1 week in culture
3. Injected adipocytes into the tail of mice
4. Euthanized mice after 5 days
5. Harvested tail skin and performed flow cytometry for Tregs (Foxp3)

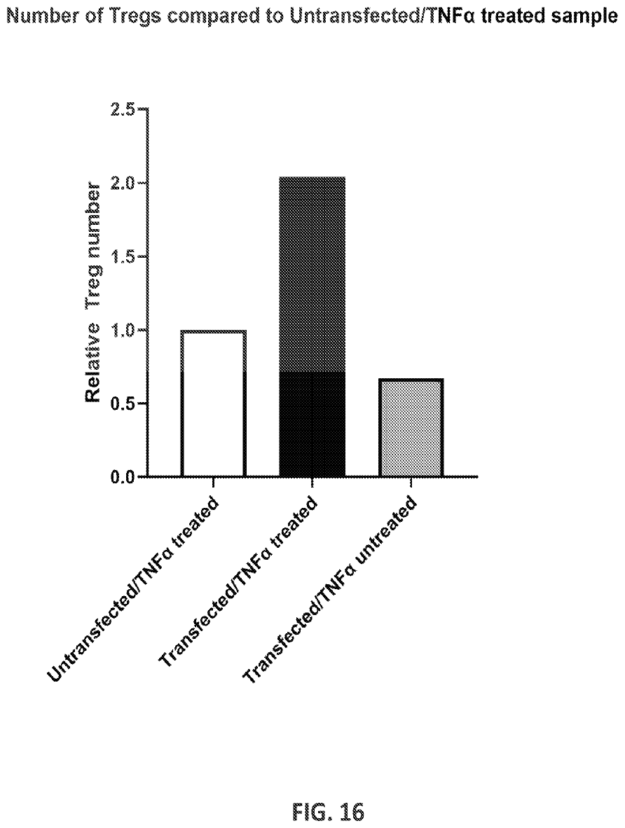

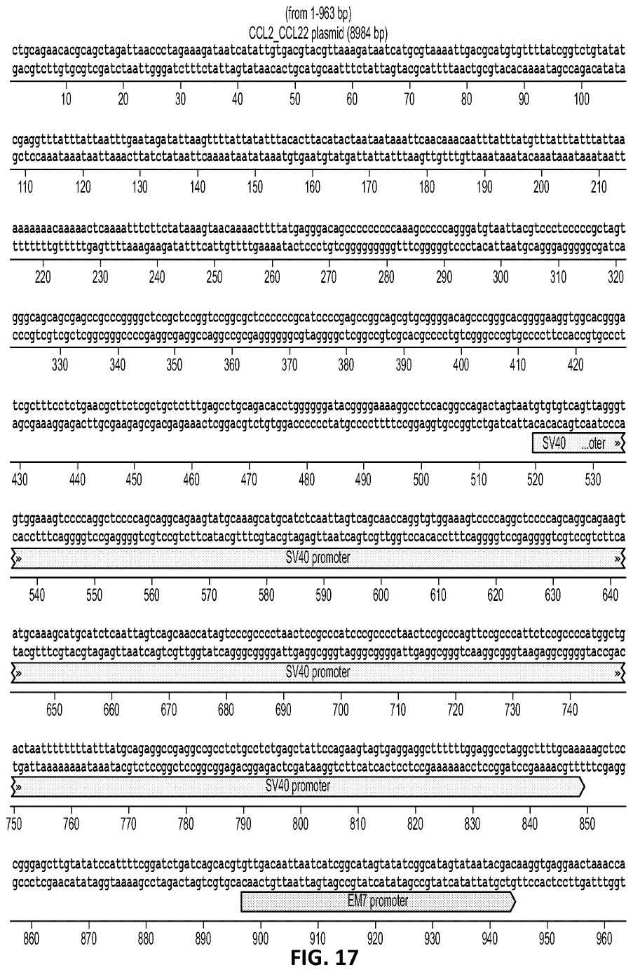

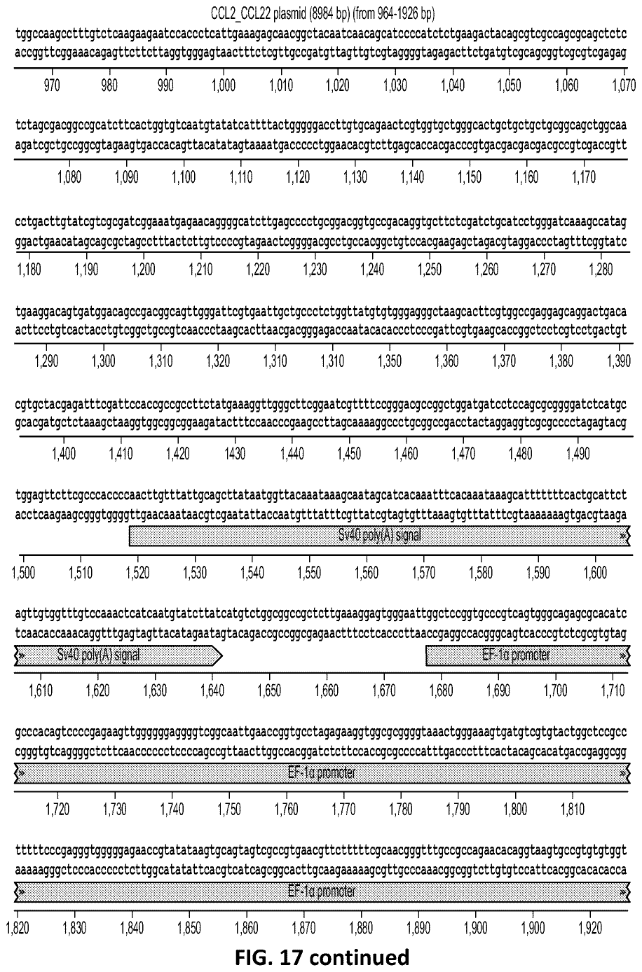

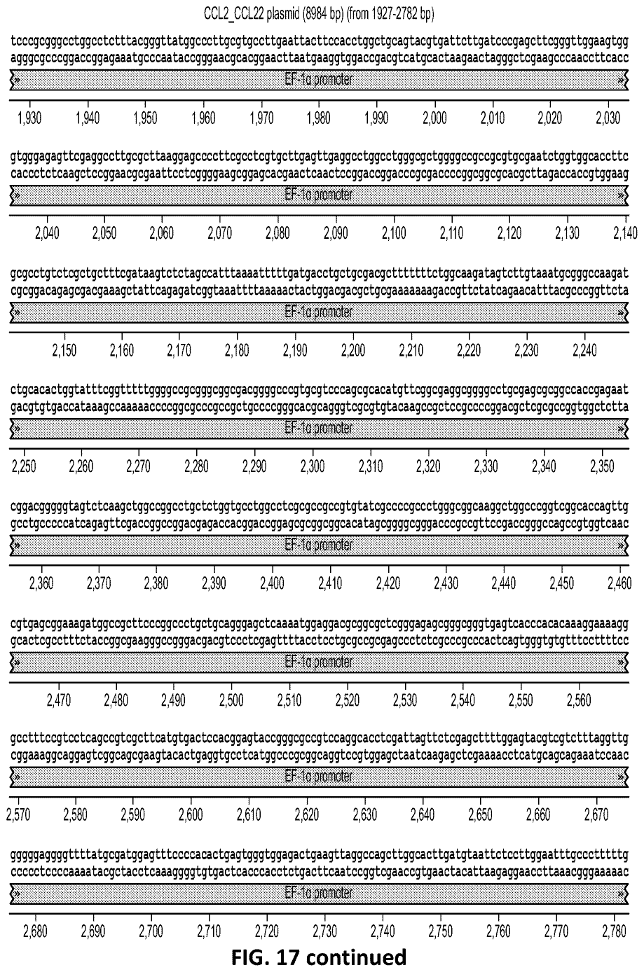

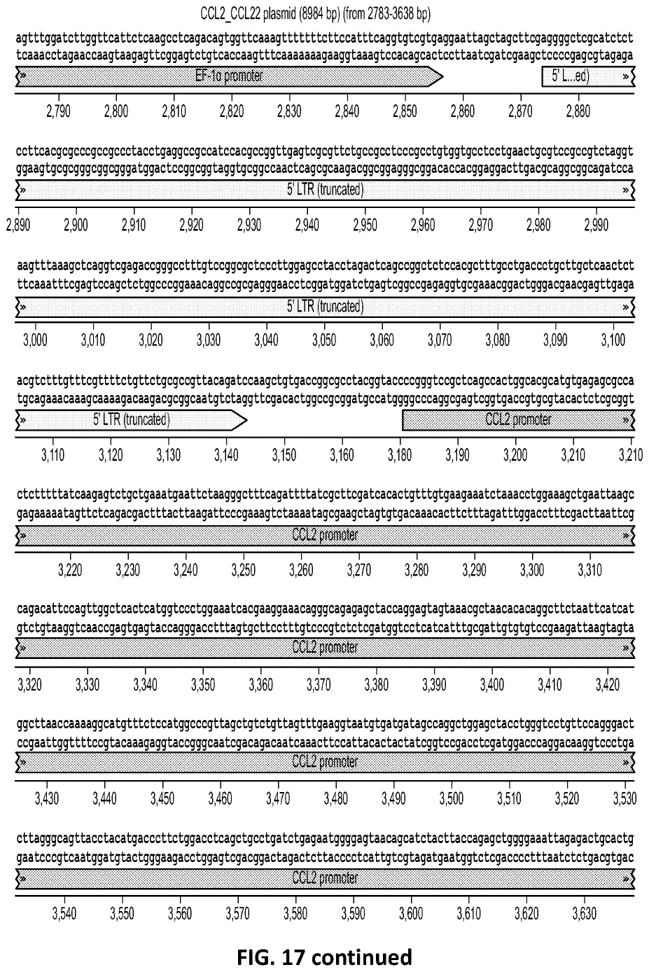

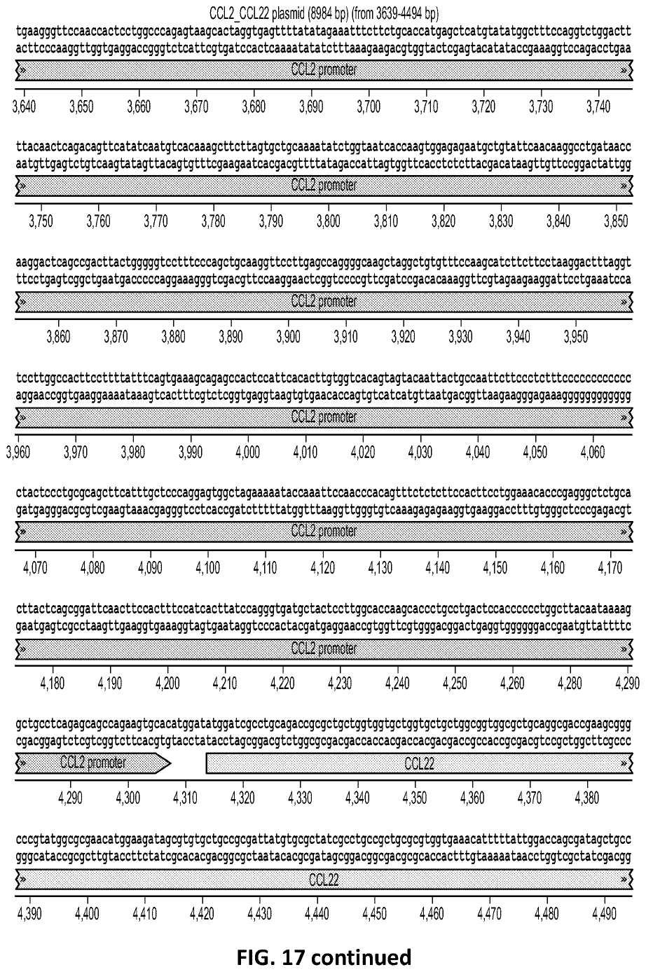

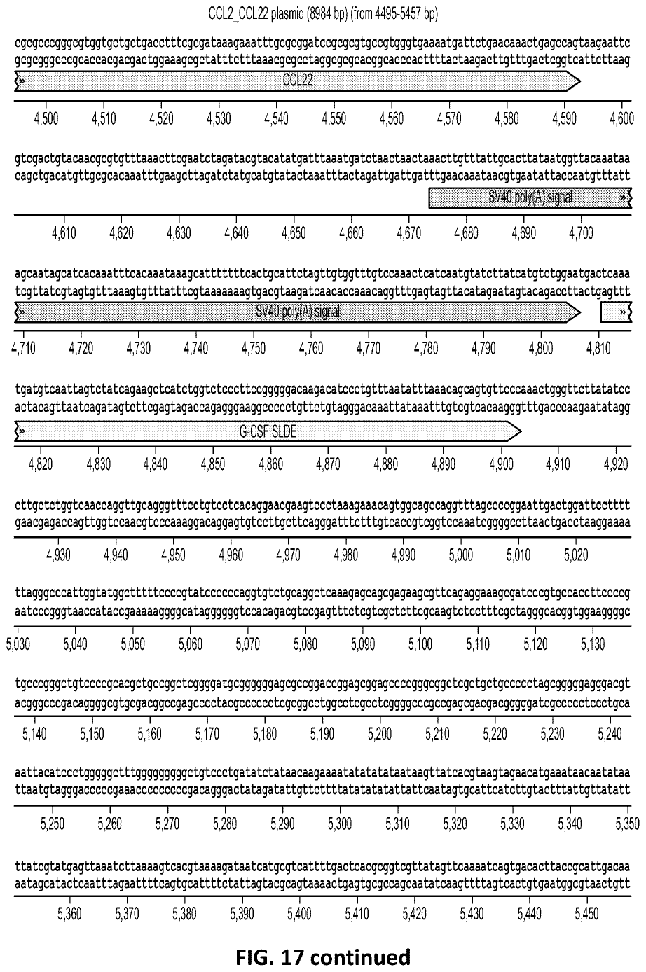

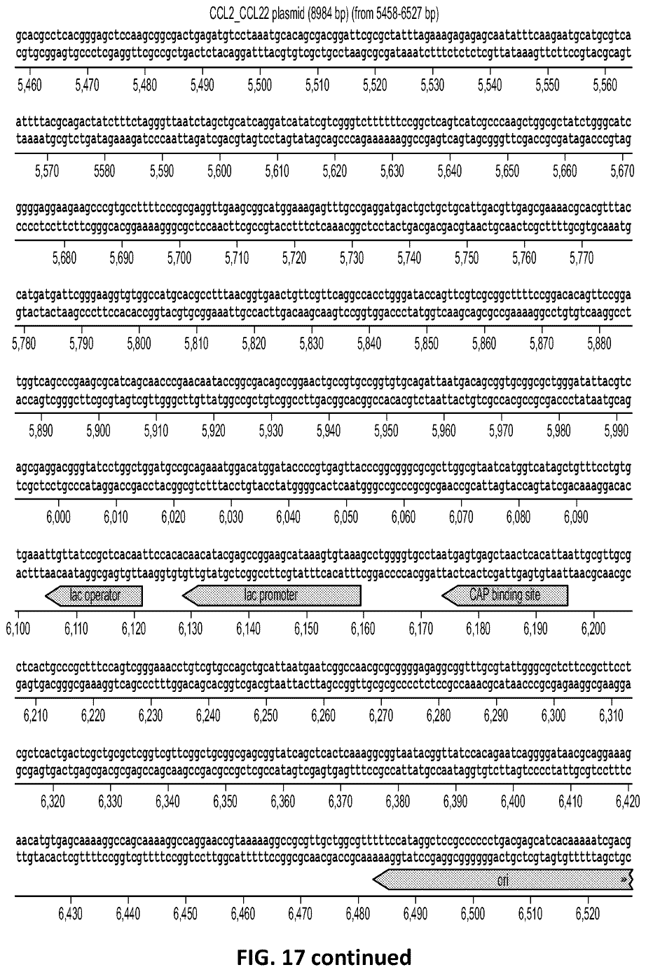

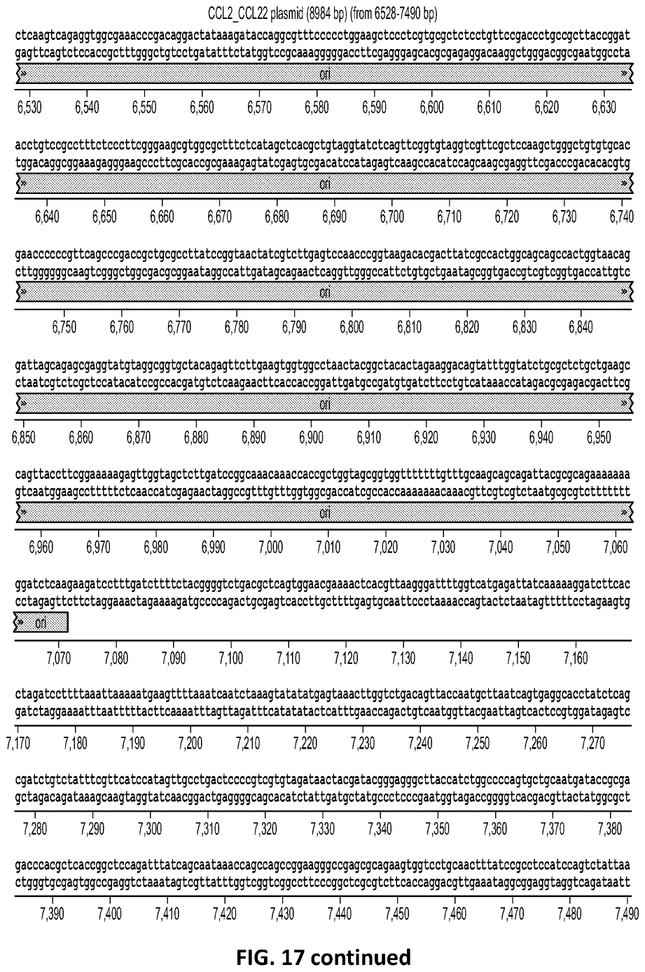

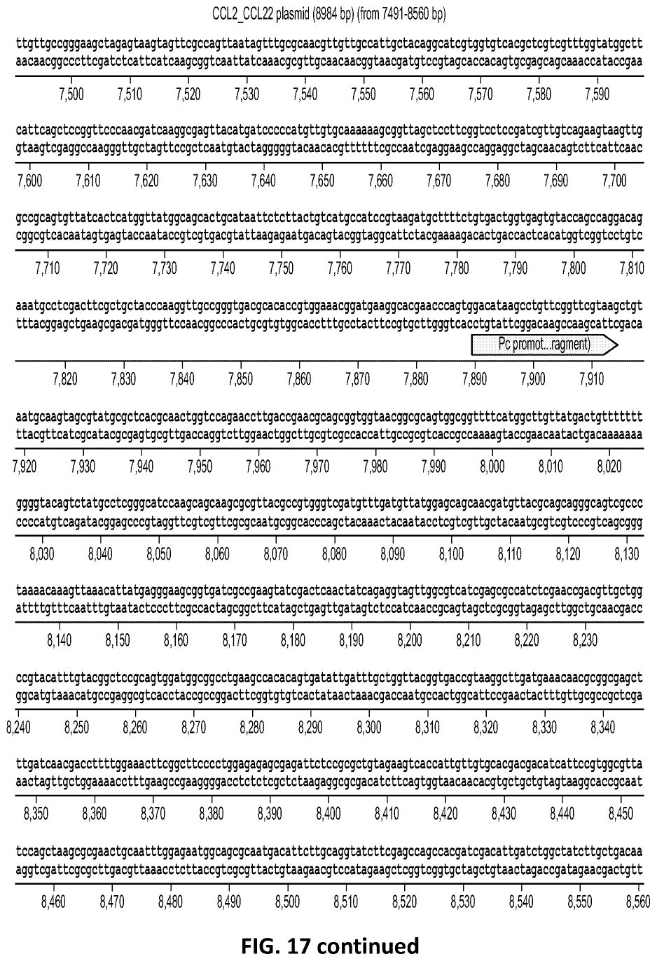

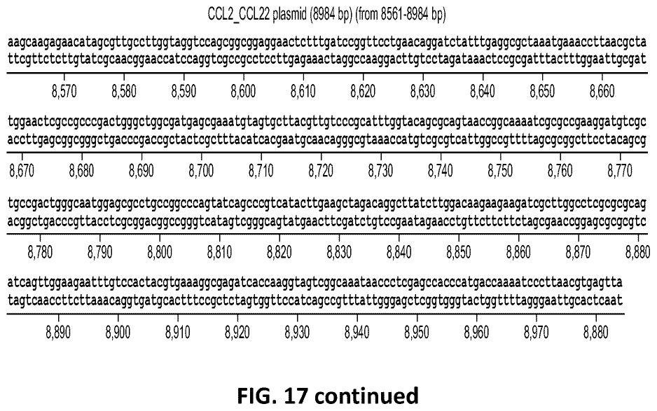

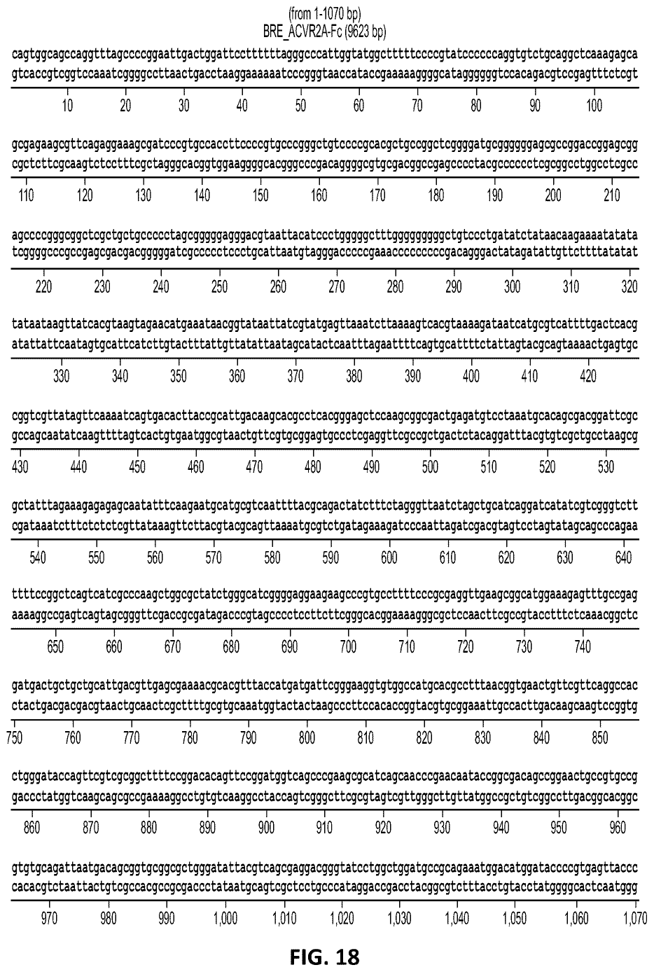

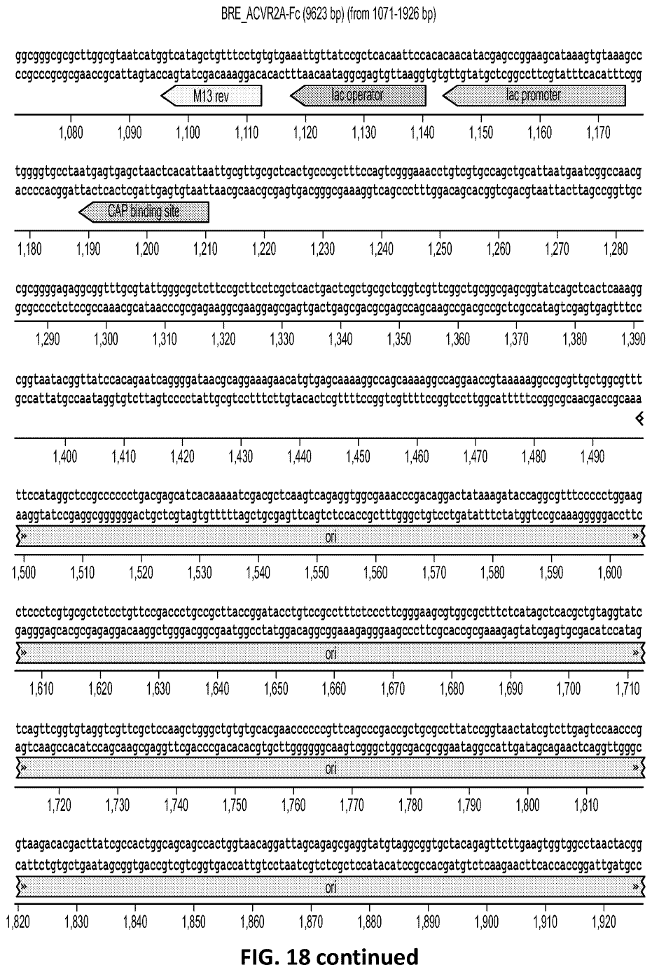

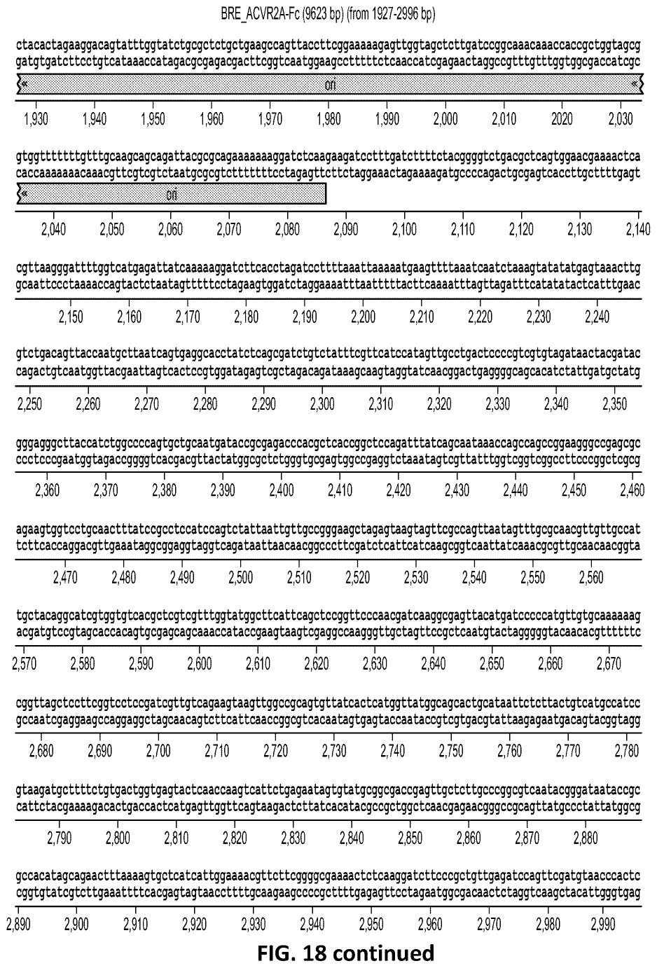

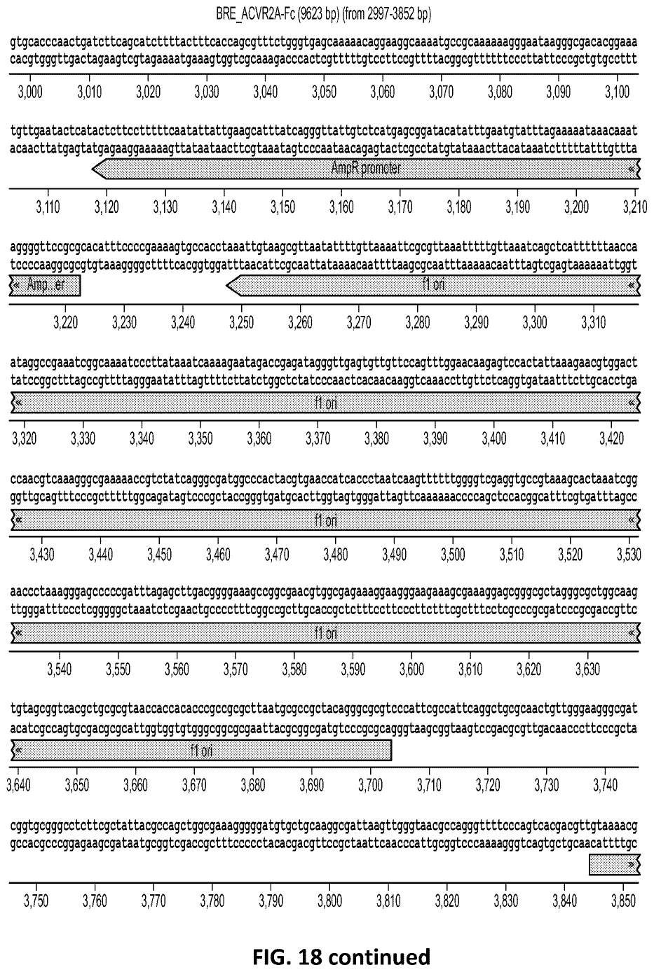

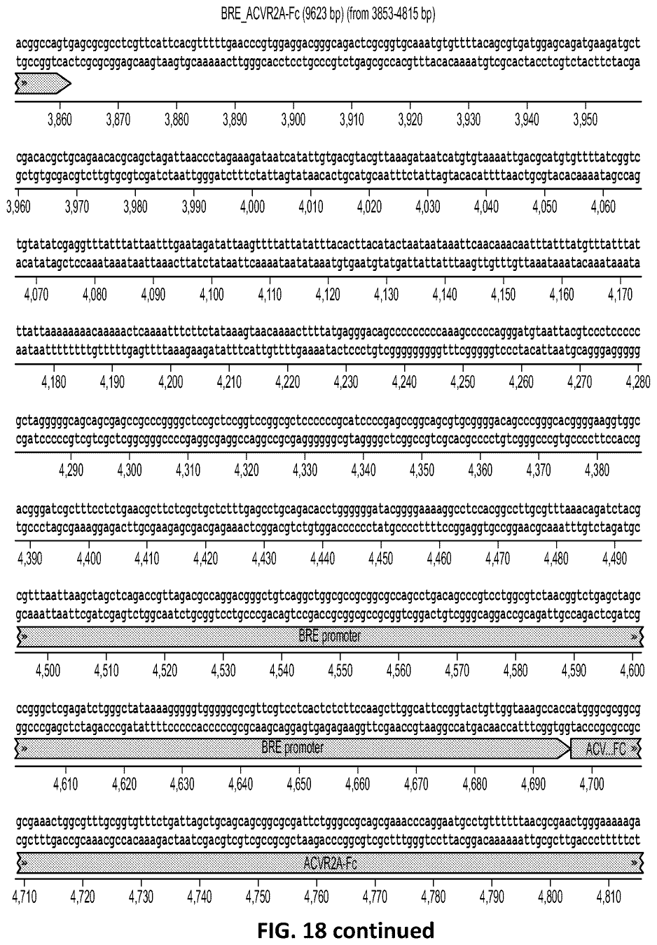

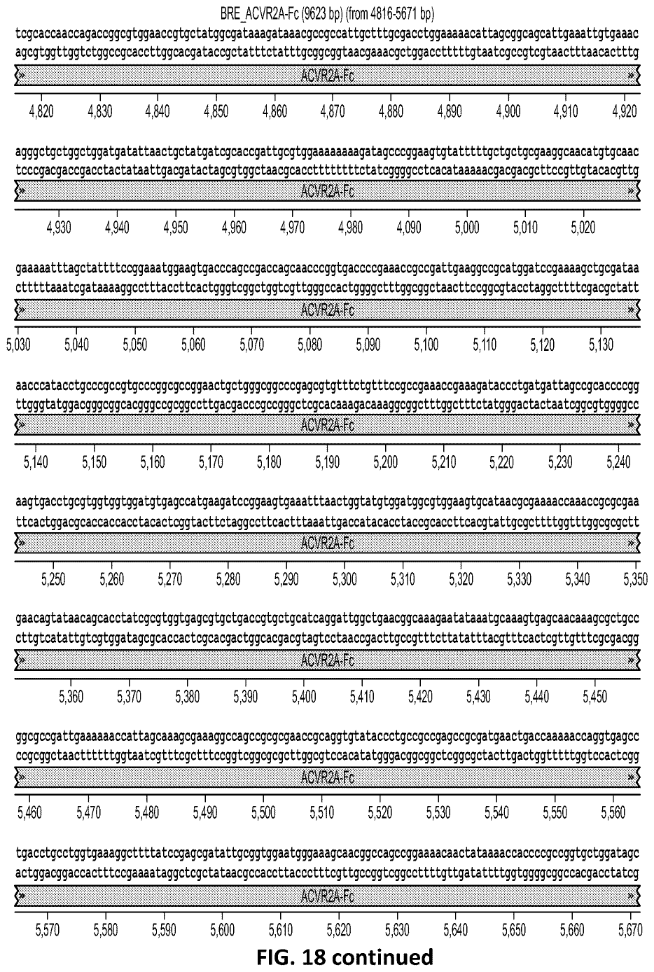

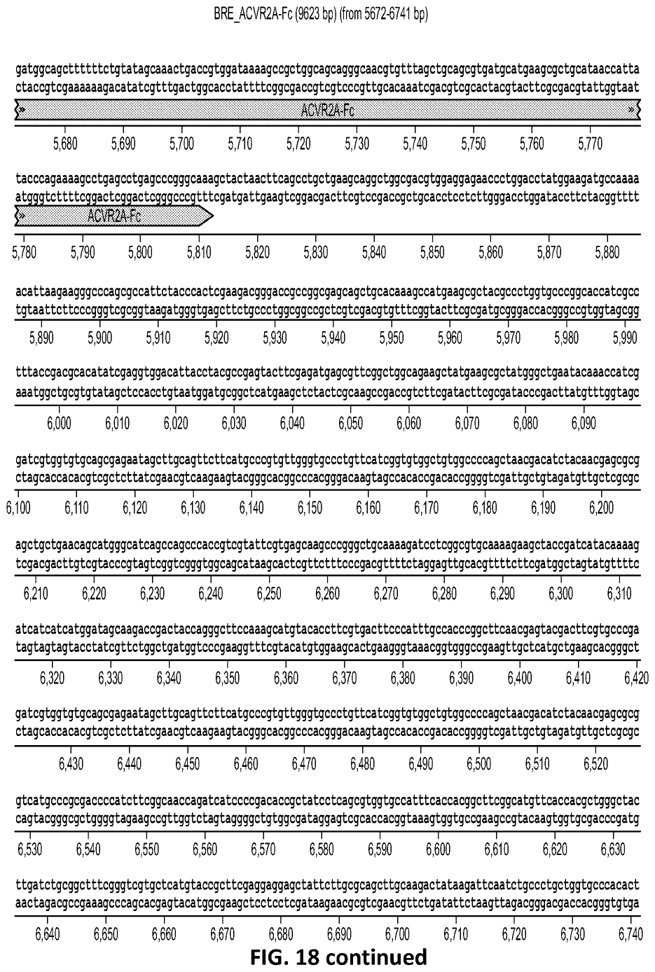

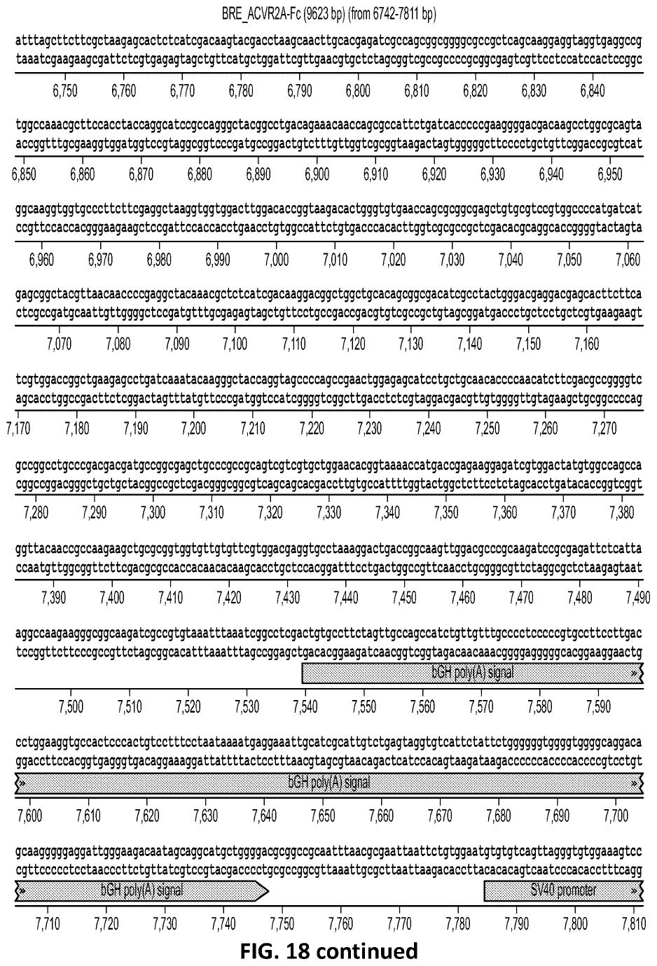

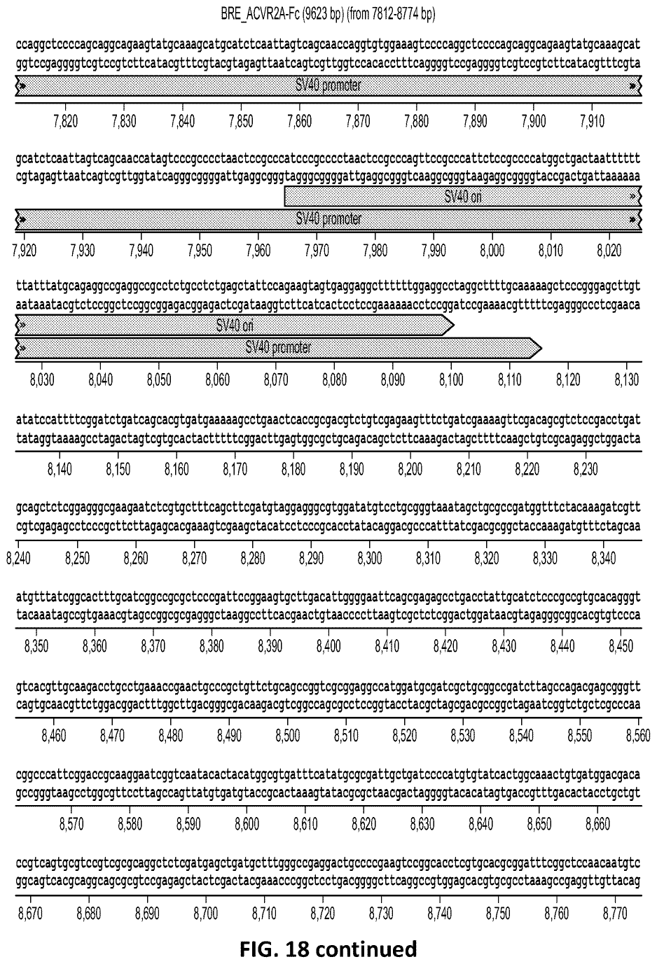

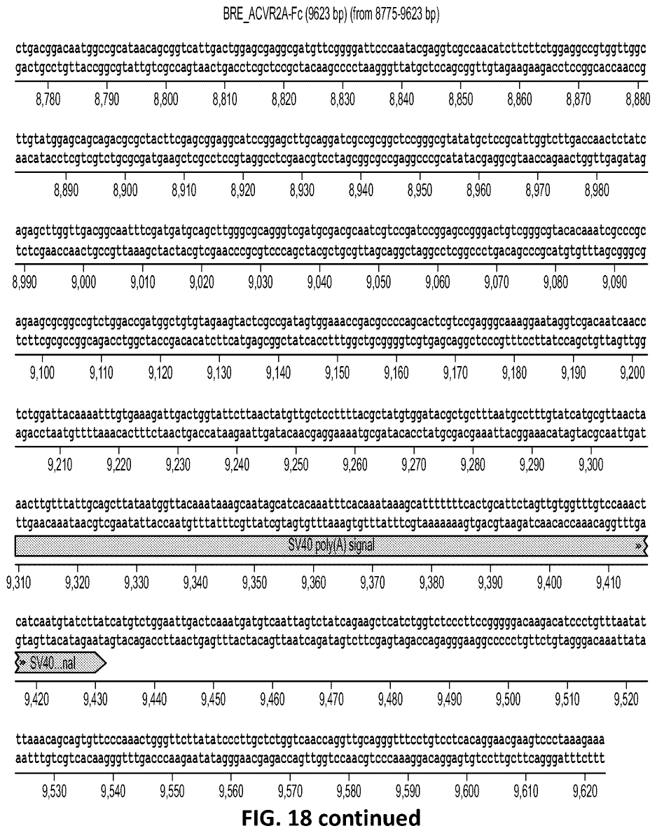

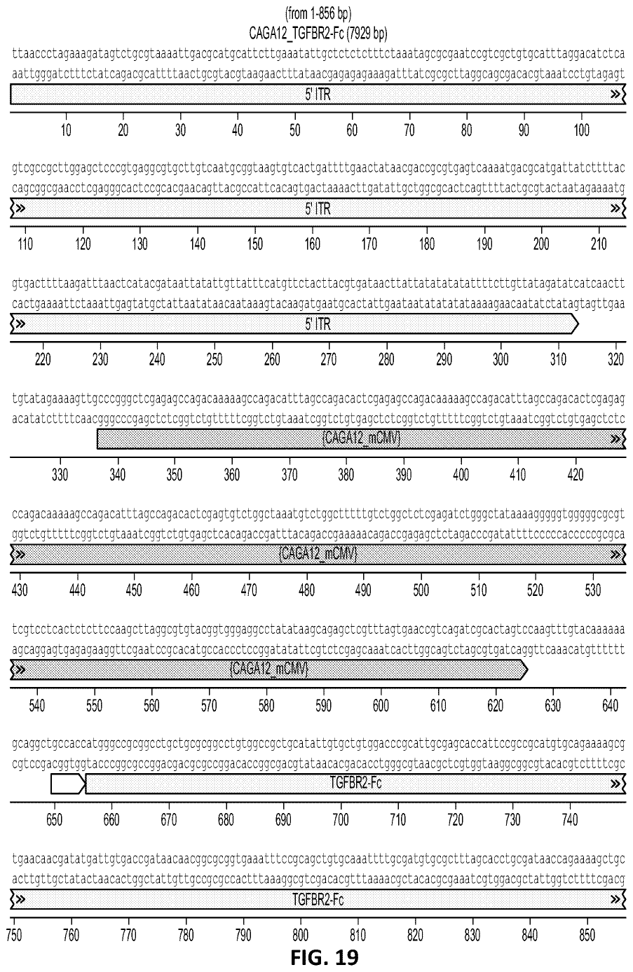

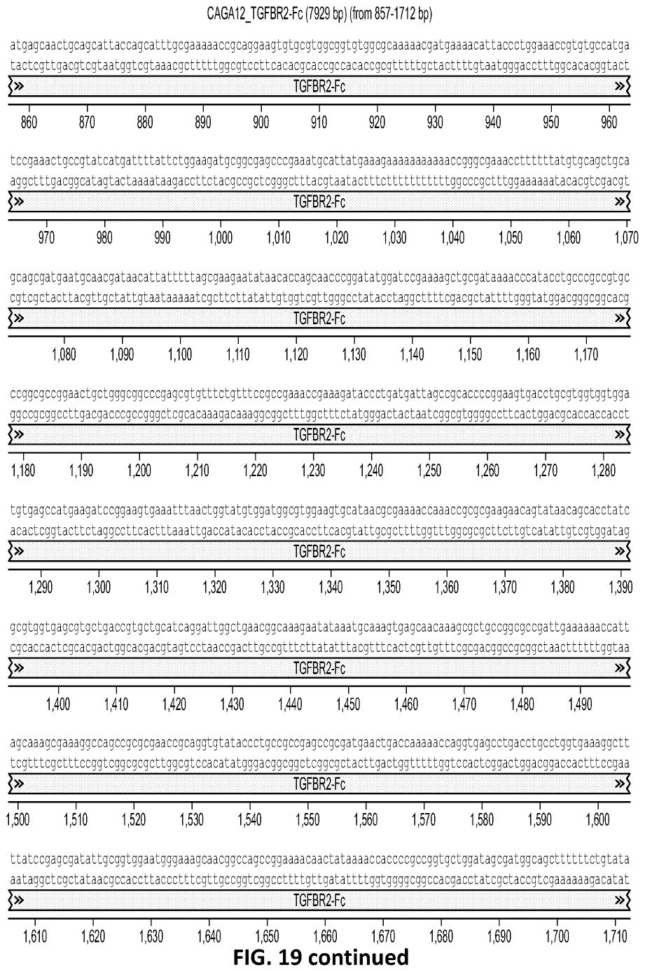

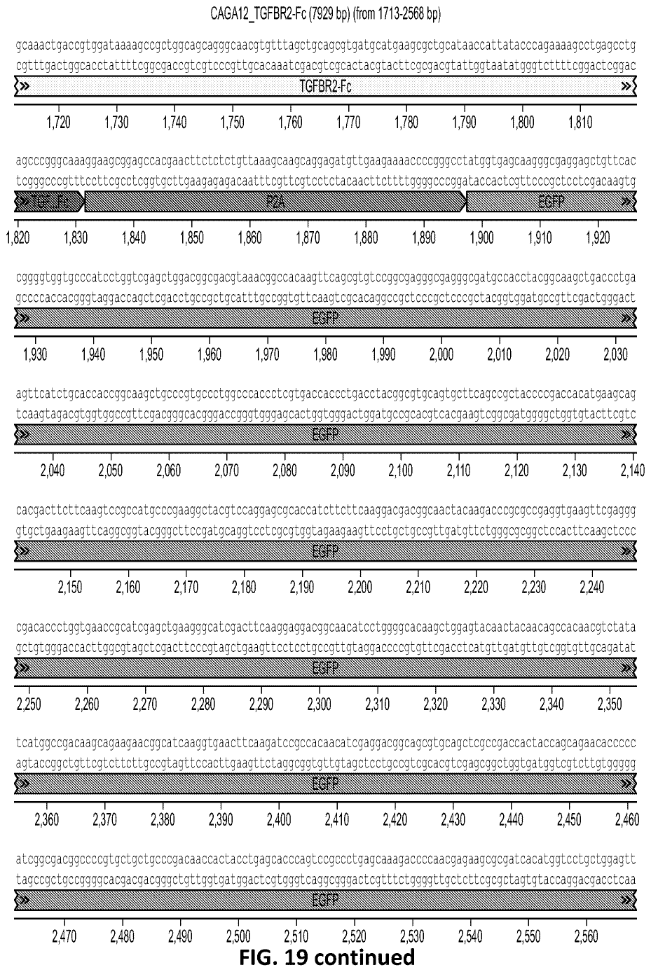

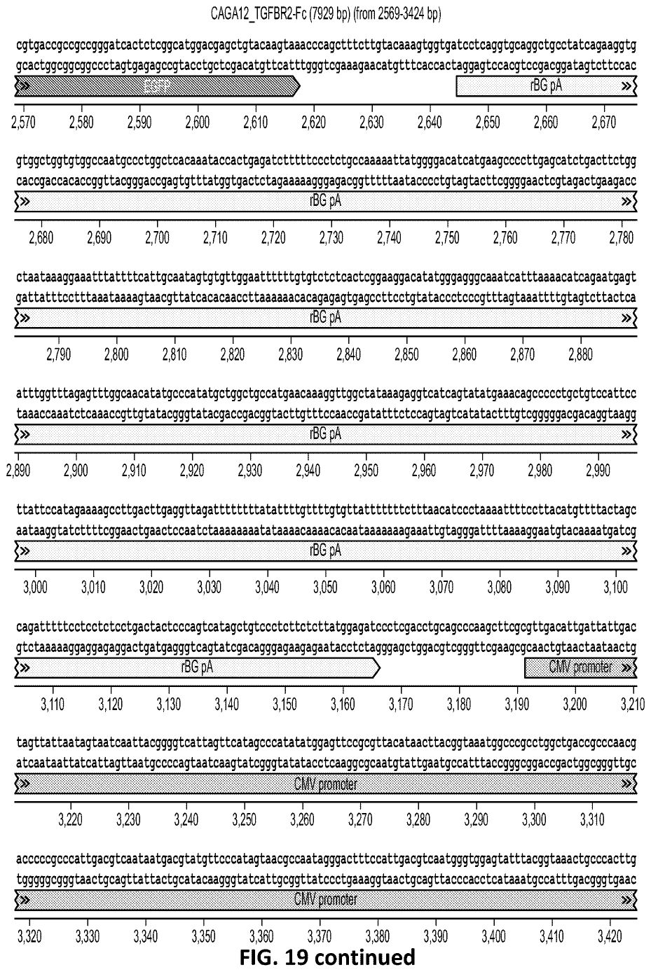

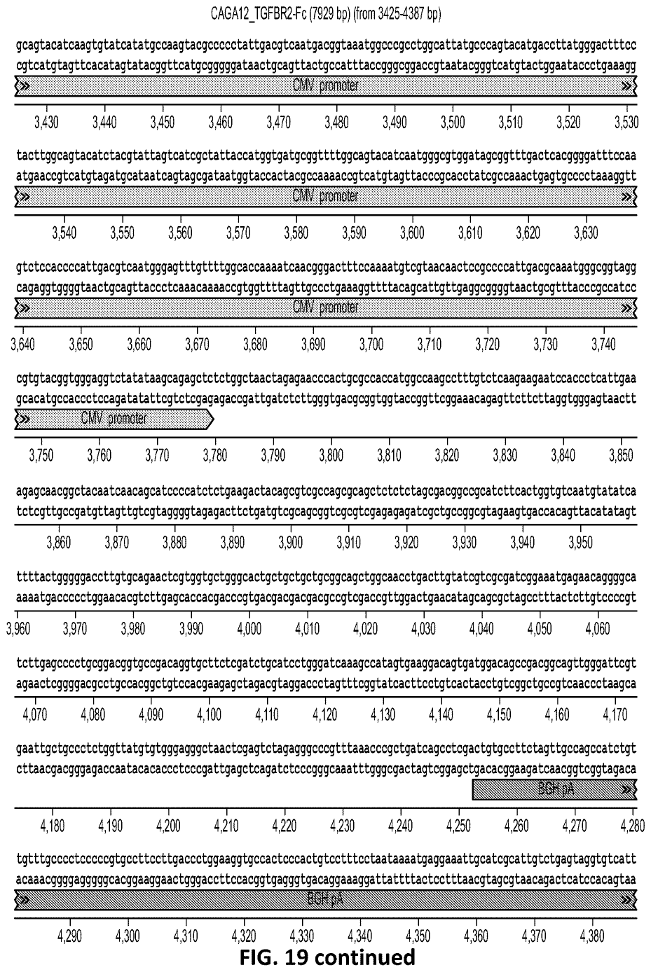

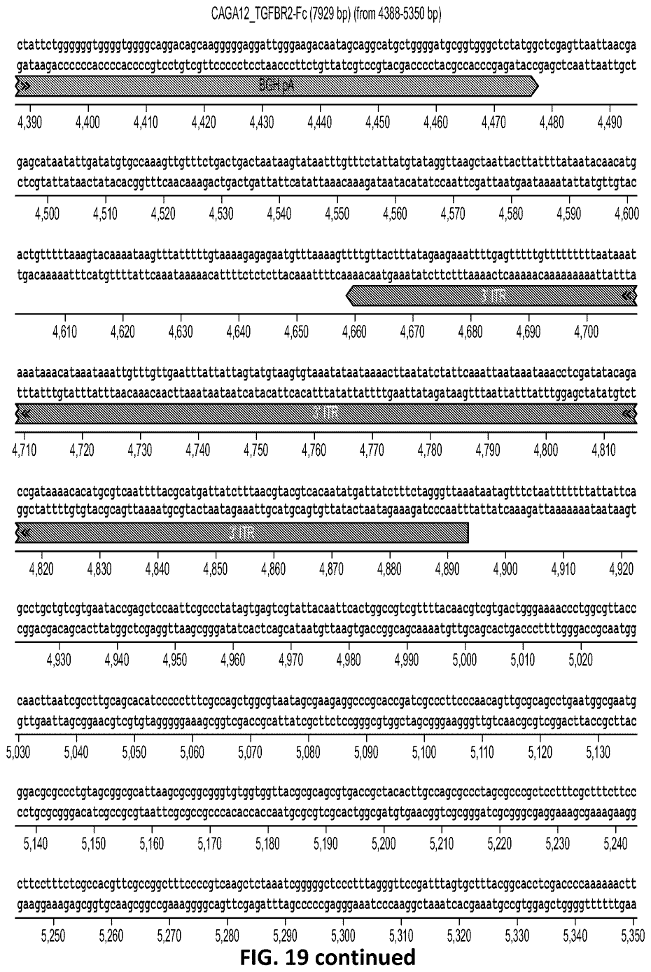

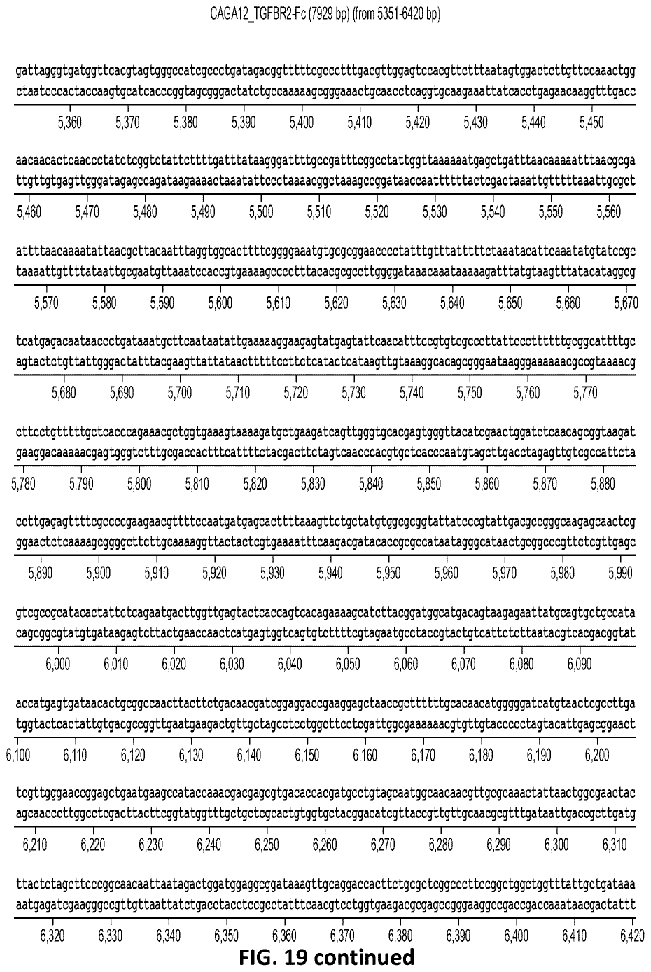

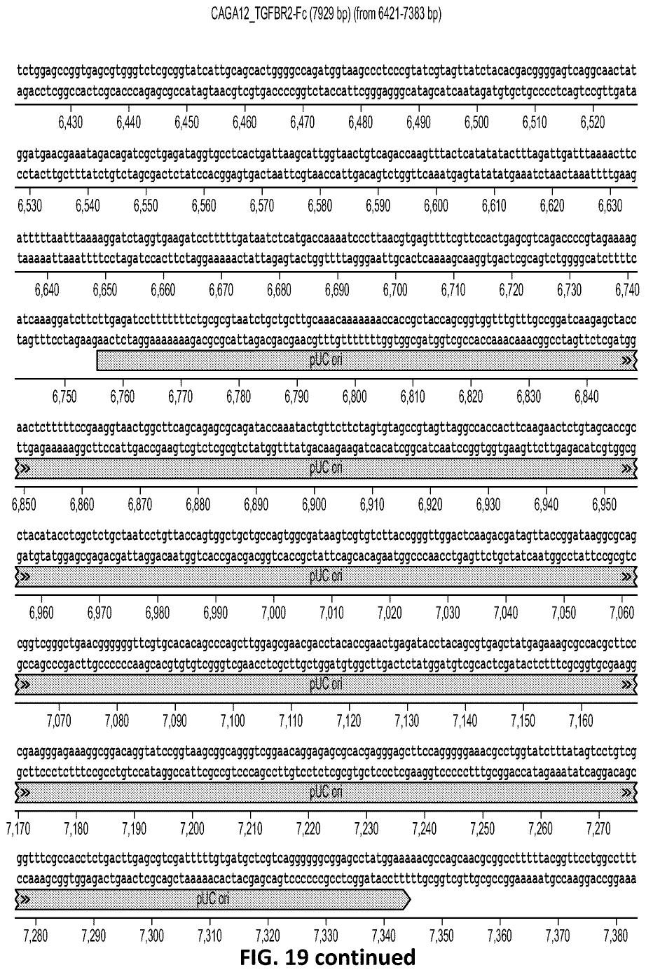

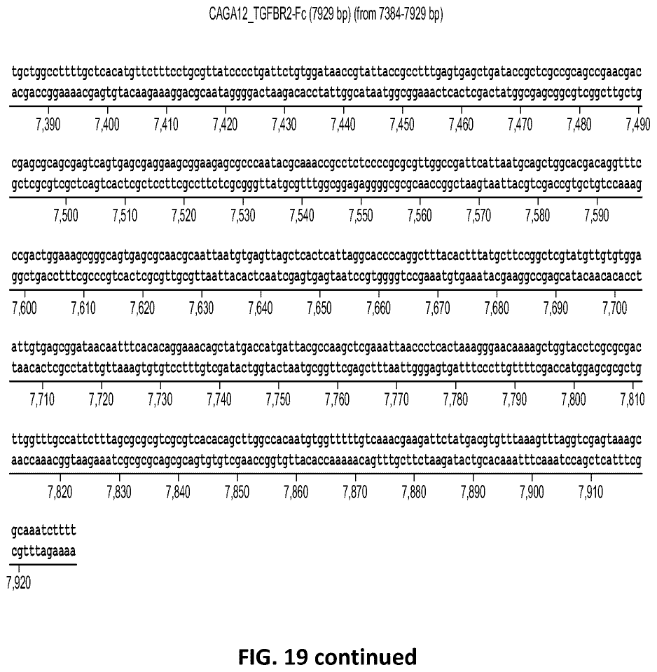

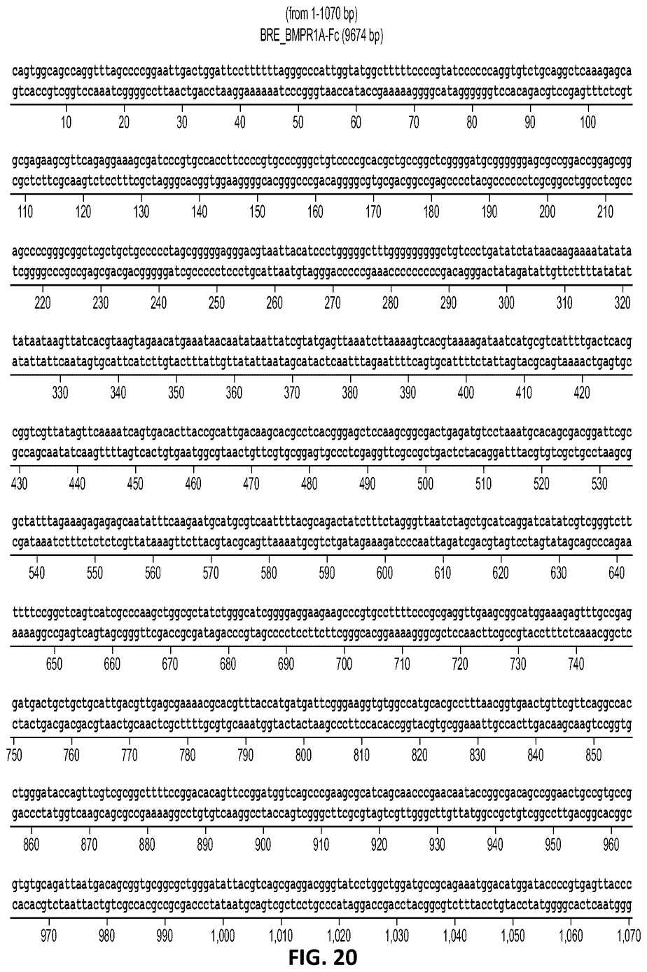

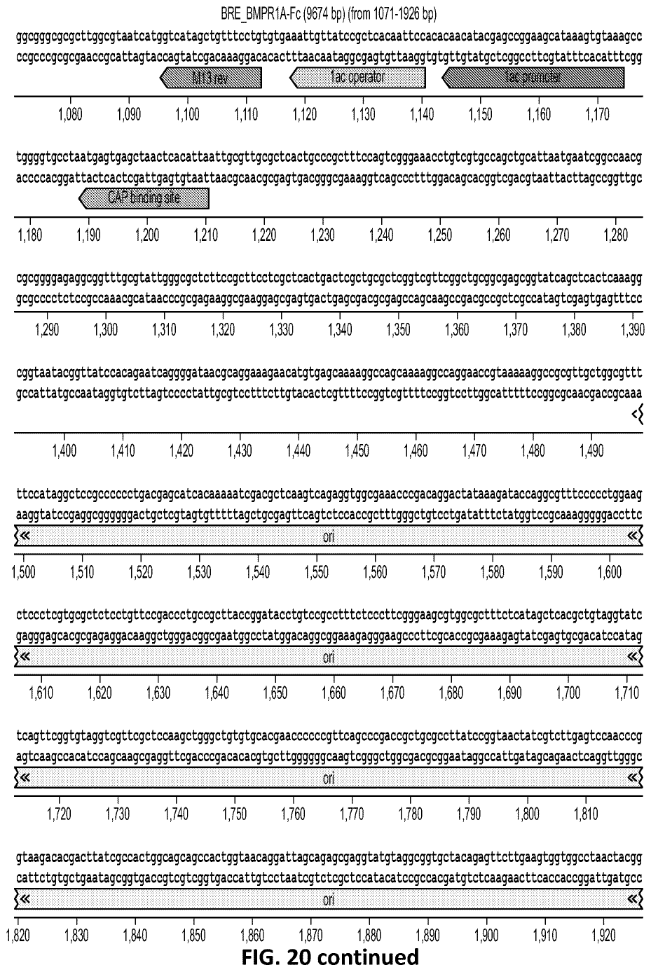

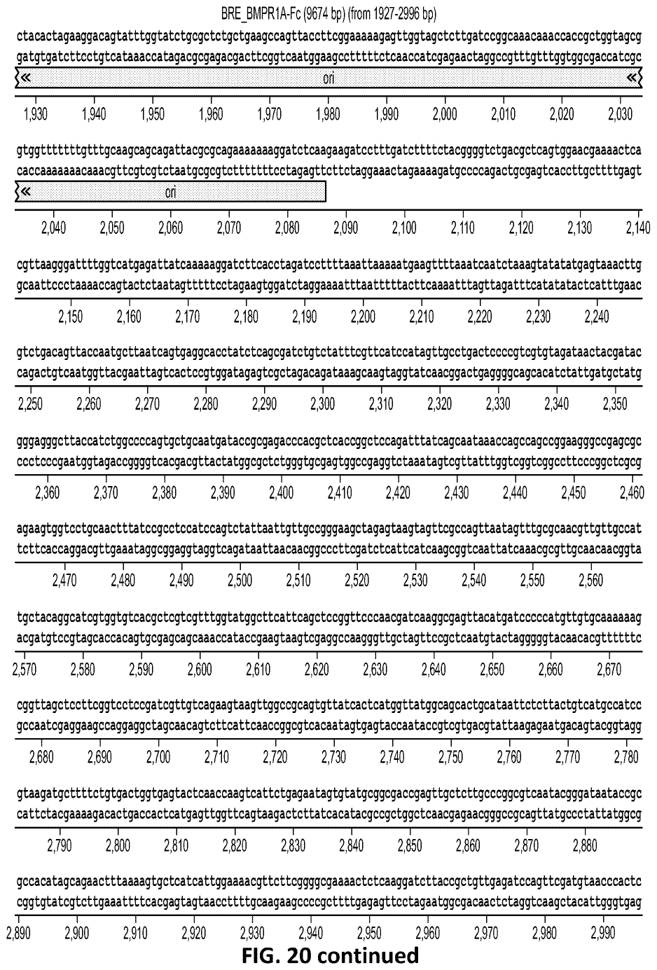

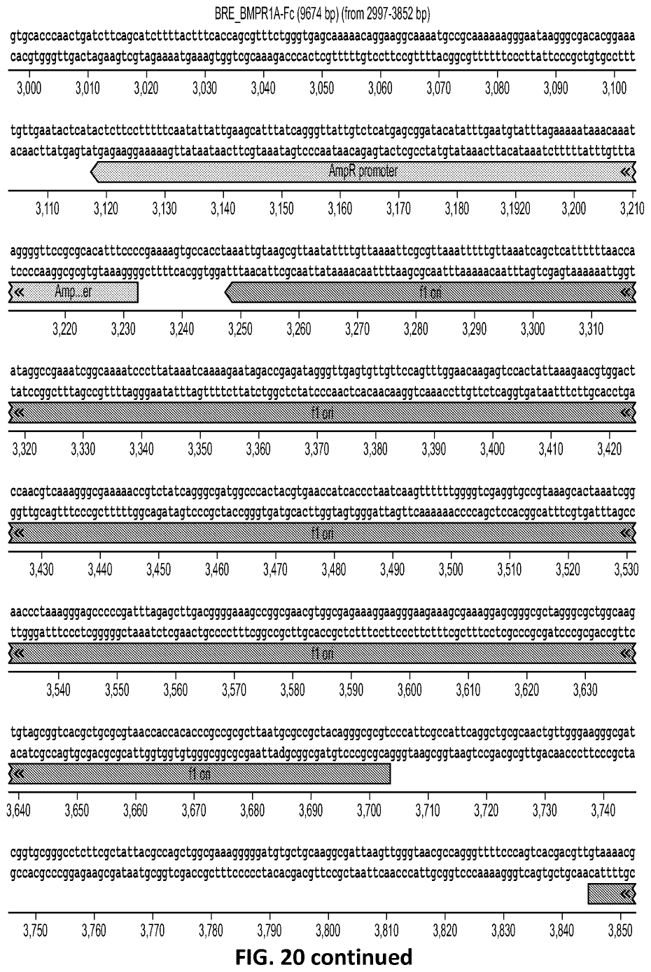

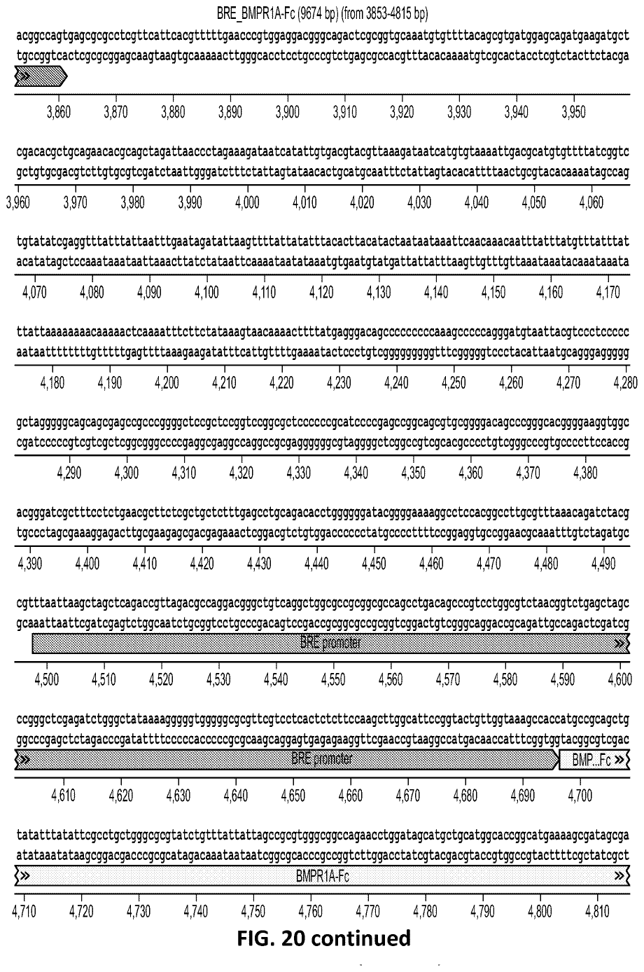

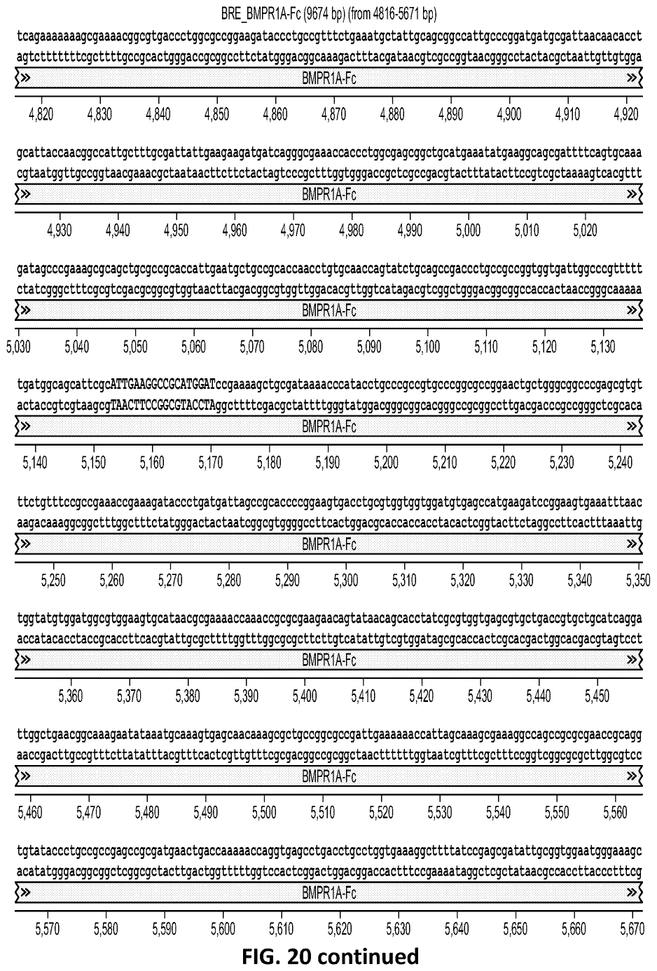

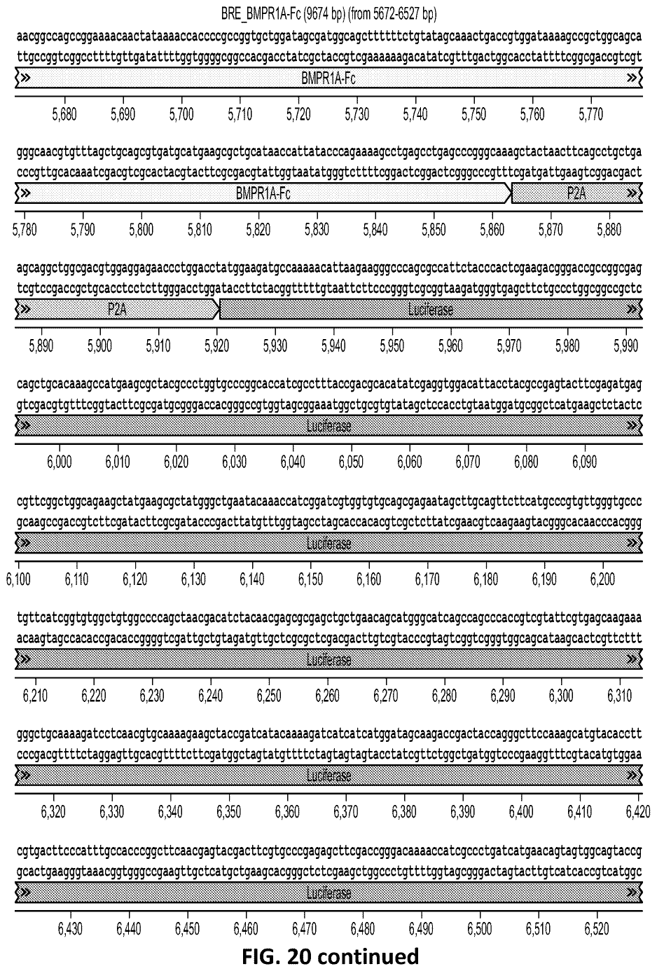

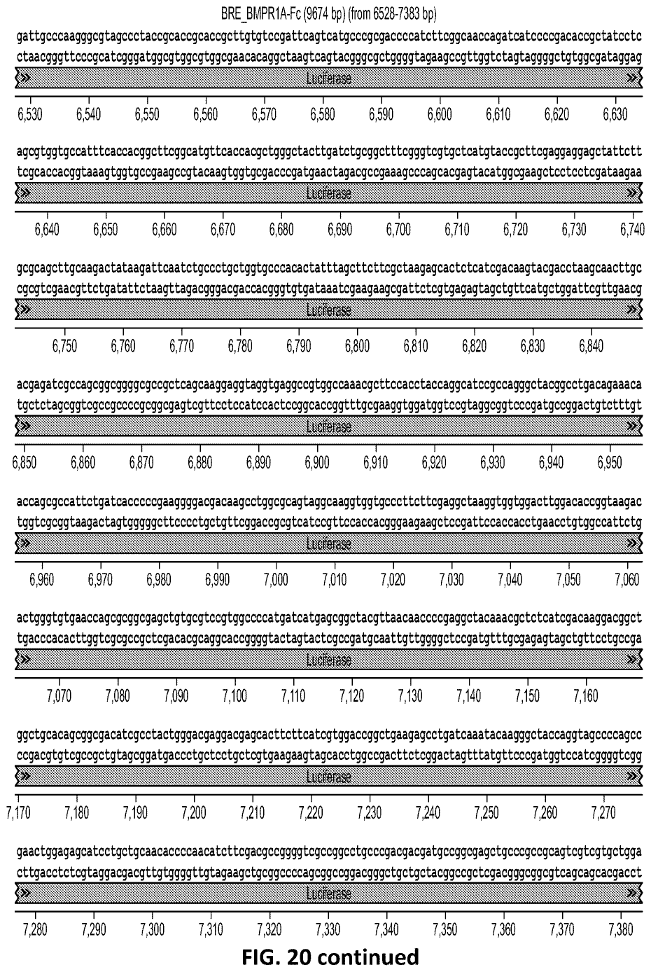

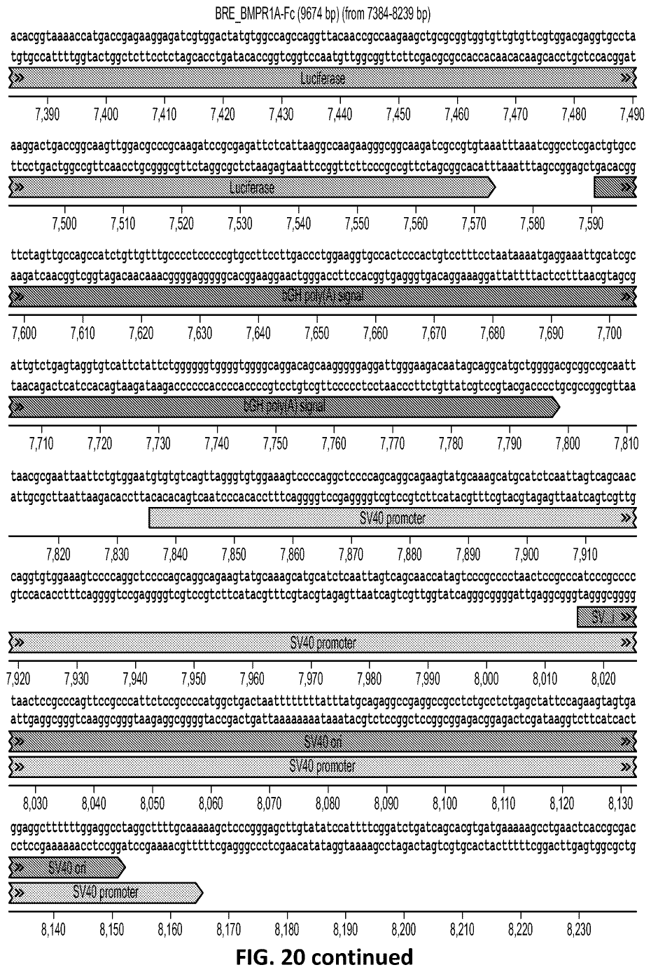

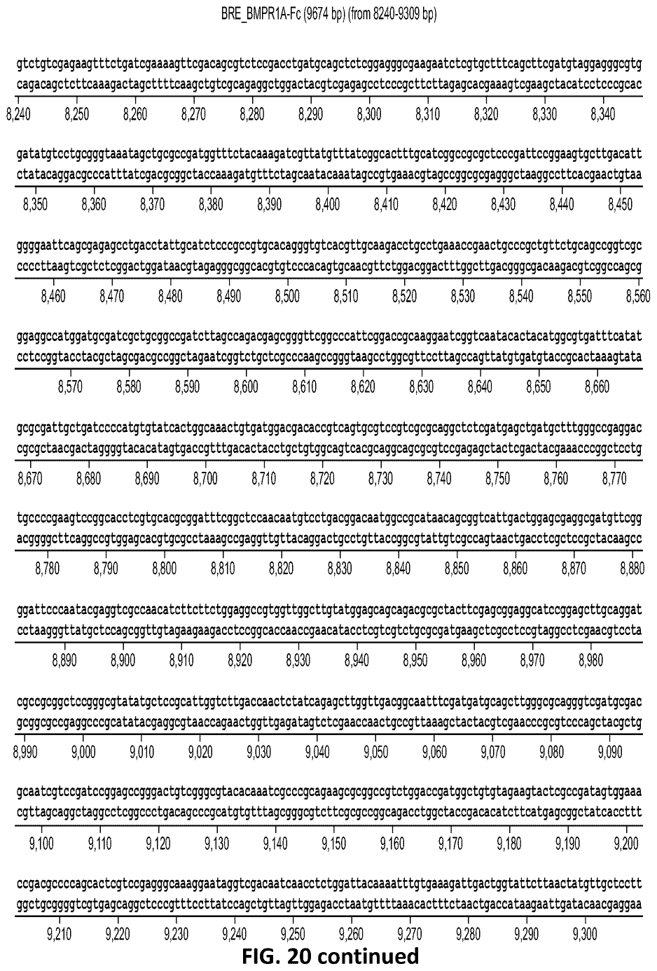

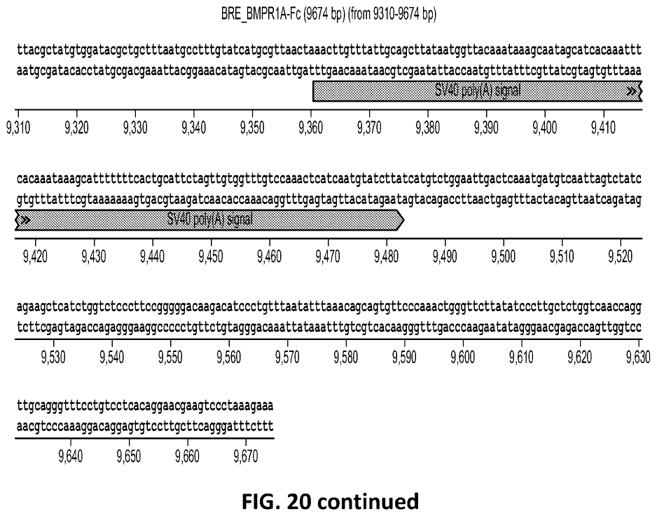

The inventors found a 2-fold increase in Treg presence in tails transplanted with adipocytes which received the plasmid and TNFα treatment relative to adipocytes transfected with null plasmid and treated with TNFα (FIG. 16). Treg number out of 10000 cells: Untransfected/TNFα treated—21; Transfected/TNFα treated—43; Transfected/TNFα untreated—14.

### Example 2

Described below is a gene circuit for treating patients with fibrodysplasia ossificans progressiva (FOP). Patients with FOP have a hyperactivating mutation in the ACVR1 gene (ACVR1 R206H mutation). As a result, cells undergo pathologic differentiation upon exposure to Activin A. The gene circuit utilizes the BMP responsive element (BRE), which is uniquely activated in cells with the ACVR1 R206H mutation in the presence of Activin A, to drive expression of ActRIIAFc. In the presence of Activin A, the cell expresses ActRIIA-Fc, thereby reducing the level of Activin A, and leading to gradual reduction in expression, rendering the gene circuit an autonomous therapy.

FOP manifests as a painful and disfiguring condition in which ectopic bony lesions form in soft tissues. Patients are affected at an early age, and develop lesions after minor inflammatory insults such as vaccinations, playground injuries, or viral illness. Eventually lesions significantly impair mobility and limit life expectancy (30-40 years).

Studies have established that global hyperactivating mutations in the type I bone morphogenetic protein (BMP) receptor ACVR1 are the underlying cause of FOP, of which the ACVR1R206H mutation is most well-studied. In contrast to wild type ACVR1, this mutation confers ACVR1 signaling hyperactivity in the presence of the pro-inflammatory, TGFβ superfamily ligand Activin A.

Using an Activin A ligand trap (ActRIIA-Fc), it has been confirmed that Activin A is a therapeutic target to prevent FOP lesions. The sporadic but life-long nature of lesion formation demands life-long systemic therapy. However, Activin A has important function in muscle maintenance and systemic inhibition may have adverse systemic consequences. An approach which enables directed, sustainable Activin A inhibition may be helpful to provide life-long treatment for patients.

Previous work has shown intense inflammatory infiltrates preceding osseous lesions in FOP. It has been shown that mTOR inhibitors, which are potent anti-inflammatory drugs, prevent FOP lesions in a mouse model. HSCs and HSC-derived cells traffic to sites of tissue inflammation and injury. Using a parabiosis mouse model, it has been shown that circulating macrophages traffic to sites of injury in trauma-induced heterotopic ossification. The presence of HSC-derived cells at the injury site of mice with FOP has been confirmed.

Economides et al. showed that HEK293/BRE-luc reporter cells which express ACVR1^{R206H}, unlike HEK293/BRE-luc with wild type ACVR1, exhibit bioluminescence upon Activin A exposure. Therefore, Activin A uniquely activates the BMP-responsive element (BRE) in cells with the ACVR1^{R206II} mutation. Therefore, the BRE promoter may be used to drive ActRIIAFc expression in ACVR1^{R206H} HSCs, thereby enabling Activin A-inducible ActRIIA-Fc expression.

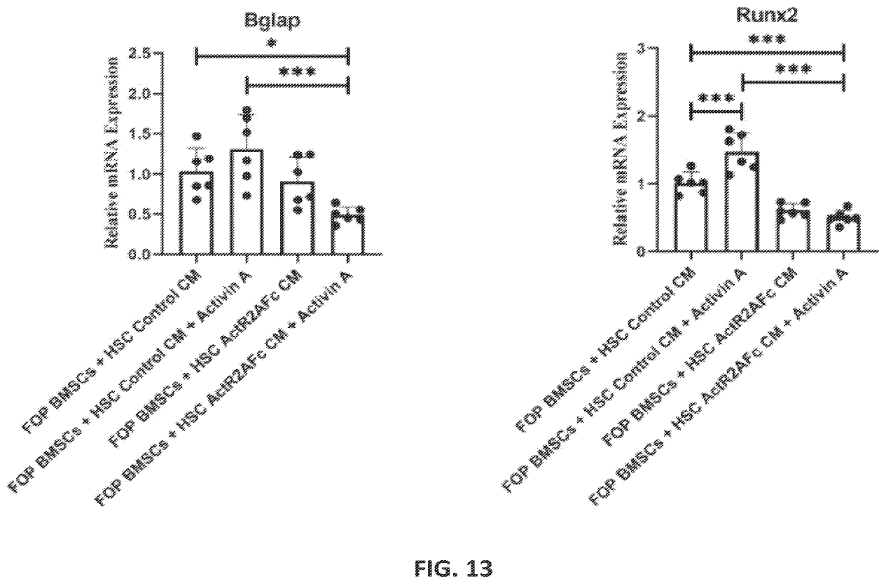

FIG. 13 demonstrates that coculturing FOP bone marrow-derived mesenchymal stem/stromal cells (BMSCs) and hematopoietic stem cells (HSCs) expressing ActR2Afc reduce expression of Bglap and Runx2. Relative mRNA expression is shown via qPCR.

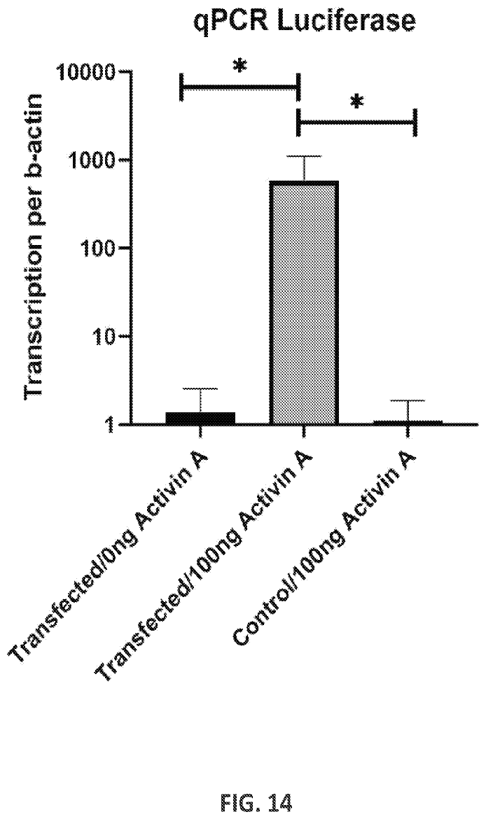

FIG. 14 demonstrates that expression of BRE-ActR2A-Fc is increased in the presence of Activin A.

### Example 3

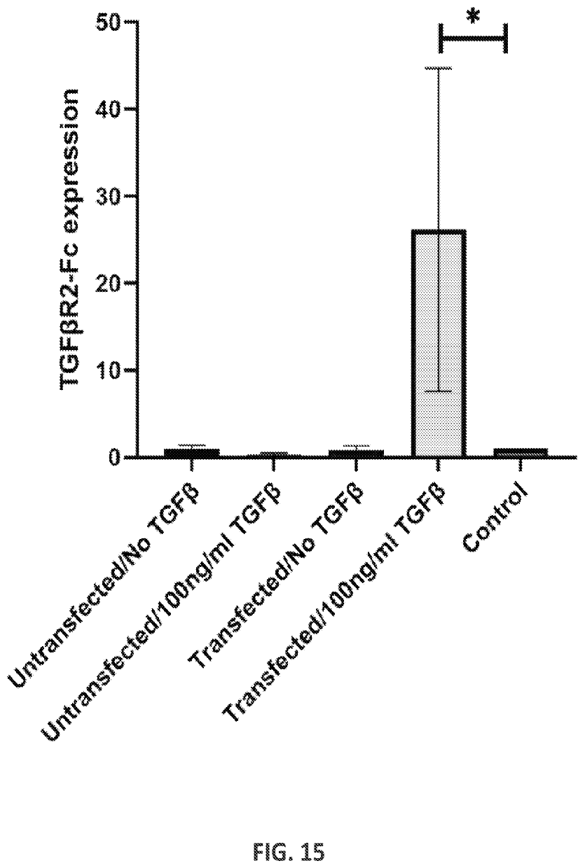

FIG. 15 demonstrates that expression of CAGA-TGFbR2-Fc is increased in the presence of TGFb (qPCR).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

TABLE 1
Informal Sequence Listing

| SEQ ID | | |
|---|---|---|
| NO: | Sequence | Description |
| 1 | CCGGGTCCGCTCAGCCACTGGCACGCATGTGAGA | CCL2-CCL22 construct |
| | GCGCCACTCTTTTTATCAAGAGTCTGCTGAAATG | |
| | AATTCTAAGGGCTTTCAGATTTTATCGCTTCGATC | |
| | ACACTGTTTGTGAAGAAATCTAAACCTGGAAAGC | |
| | TGAATTAAGCCAGACATTCCAGTTGGCTCACTCA | |
| | TGGTCCCTGGAAATCACGAAGGAAACAGGGCAG | |
| | AGAGCTACCAGGAGTAGTAAACGCTAACACACA | |
| | GGCTTCTAATTCATCATGGCTTAACCAAAAGGCA | |
| | TGTTTCTCCATGGCCCGTTAGCTGTCTGTTAGTTT | |
| | GAAGGTAATGTGATGATAGCCAGGCTGGAGCTAC | |
| | CTGGGTCCTGTTCCAGGGACTCTTAGGGCAGTTA | |
| | CCTACATGACCCTTCTGGACCTCAGCTGCCTGATC | |
| | TGAGAATGGGGAGTAACAGCATCTACTTACCAGA | |
| | GCTGGGGAAATTAGAGACTGCACTGTGAAGGGTT | |
| | CCAACCACTCCTGGCCCAGAGTAAGCACTAGGTG | |
| | AGTTTTATATAGAAATTTCTTCTGCACCATGAGCT | |
| | CATGTATATGGCTTTCCAGGTCTGGACTTTTACAA | |
| | CTCAGACAGTTCATATCAATGTCACAAAGCTTCT | |
| | TAGTGCTGCAAAATATCTGGTAATCACCAAGTGG | |
| | AGAGAATGCTGTATTCAACAAGGCCTGATAACCA | |
| | AGGACTCAGCCGACTTACTGGGGGTCCTTTCCCA | |
| | GCTGCAAGGTTCCTTGAGCCAGGGGCAAGCTAGG | |
| | CTGTGTTTCCAAGCATCTTCTTCCTAAGGACTTTA | |
| | GGTTCCTTGGCCACTTCCTTTTATTTCAGTGAAAG | |
| | CAGAGCCACTCCATTCACACTTGTGGTCACAGTA | |
| | GTACAATTACTGCCAATTCTTCCCTCTTTCCCCCC | |
| | CCCCCCCTACTCCCTGCGCAGCTTCATTTGCTCCC | |
| | AGGAGTGGCTAGAAAAATACCAAATTCCAACCC | |
| | ACAGTTTCTCTCTTCCACTTCCTGGAAACACCCGA | |
| | GGGCTCTGCACTTACTCAGCGGATTCAACTTCCA | |
| | CTTTCCATCACTTATCCAGGGTGATGCTACTCCTT | |
| | GGCACCAAGCACCCTGCCTGACTCCACCCCCCTG | |
| | GCTTACAATAAAAGGCTGCCTCAGAGCAGCCAGA | |
| | AGTGCACATGGATATGGATCGCCTGCAGACCGCG | |
| | CTGCTGGTGGTGCTGGTGCTGCTGGCGGTGGCGC | |
| | TGCAGGCGACCGAAGCGGGCCCGTATGGCGCGA | |
| | ACATGGAAGATAGCGTGTGCTGCCGCGATTATGT | |
| | GCGCTATCGCCTGCCGCTGCGCGTGGTGAAACAT | |
| | TTTTATTGGACCAGCGATAGCTGCCCGCGCCCGG | |
| | GCGTGGTGCTGCTGACCTTTCGCGATAAAGAAAT | |
| | TTGCGCGGATCCGCGCGTGCCGTGGGTGAAAATG | |
| | ATTCTGAACAAACTGAGCCAGT | |
| 2 | CTGCAGAACACGCAGCTAGATTAACCCTAGAAAG | CCL2-CCL22 |
| | ATAATCATATTGTGACGTACGTTAAAGATAATCA | transposon vector |
| | TGCGTAAAATTGACGCATGTGTTTTATCGGTCTGT | (CCL2-CCL22 construct |
| | ATATCGAGGTTTATTTATTAATTTGAATAGATATT | underlined, CCL2 |
| | AAGTTTTATTATATTTACACTTACATACTAATAAT | promoter bold, CCL22 |
| | AAATTCAACAAACAATTTATTTATGTTTATTTATT | gene italicized) |
| | TATTAAAAAAAAACAAAAACTCAAAATTTCTTCT | |
| | ATAAAGTAACAAAACTTTTATGAGGGACAGCCCC | |
| | CCCCCAAAGCCCCCAGGGATGTAATTACGTCCCT | |
| | CCCCCGCTAGTGGGCAGCAGCGAGCCGCCCGGG | |
| | GCTCCGCTCCGGTCCGGCGCTCCCCCCGCATCCC | |
| | CGAGCCGGCAGCGTGCGGGGACAGCCCGGGCAC | |
| | GGGGAAGGTGGCACGGGATCGCTTTCCTCTGAAC | |
| | GCTTCTCGCTGCTCTTTGAGCCTGCAGACACCTGG | |
| | GGGGATACGGGGAAAAGGCCTCCACGGCCAGAC | |
| | TAGTAATGTGTGTCAGTTAGGGTGTGGAAAGTCC | |
| | CCAGGCTCCCCAGCAGGCAGAAGTATGCAAAGC | |
| | ATGCATCTCAATTAGTCAGCAACCAGGTGTGGAA | |
| | AGTCCCCAGGCTCCCCAGCAGGCAGAAGTATGCA | |
| | AAGCATGCATCTCAATTAGTCAGCAACCATAGTC | |
| | CCGCCCCTAACTCCGCCCATCCCGCCCCTAACTCC | |
| | GCCCAGTTCCGCCCATTCTCCGCCCCATGGCTGA | |
| | CTAATTTTTTTTATTTATGCAGAGGCCGAGGCCGC | |
| | CTCTGCCTCTGAGCTATTCCAGAAGTAGTGAGGA | |
| | GGCTTTTTTGGAGGCCTAGGCTTTTGCAAAAAGC | |
| | TCCCGGGAGCTTGTATATCCATTTTCGGATCTGAT | |
| | CAGCACGTGTTGACAATTAATCATCGGCATAGTA | |
| | TATCGGCATAGTATAATACGACAAGGTGAGGAAC | |
| | TAAACCATGGCCAAGCCTTTGTCTCAAGAAGAAT | |
| | CCACCCTCATTGAAAGAGCAACGGCTACAATCAA | |
| | CAGCATCCCCATCTCTGAAGACTACAGCGTCGCC | |
| | AGCGCAGCTCTCTCTAGCGACGGCCGCATCTTCA | |
| | CTGGTGTCAATGTATATCATTTTACTGGGGGACCT | |
| | TGTGCAGAACTCGTGGTGCTGGGCACTGCTGCTG | |
| | CTGCGGCAGCTGGCAACCTGACTTGTATCGTCGC | |
| | GATCGGAAATGAGAACAGGGGCATCTTGAGCCC | |
| | CTGCGGACGGTGCCGACAGGTGCTTCTCGATCTG | |
| | CATCCTGGGATCAAAGCCATAGTGAAGGACAGTG | |
| | ATGGACAGCCGACGGCAGTTGGGATTCGTGAATT | |
| | GCTGCCCTCTGGTTATGTGTGGGAGGGCTAAGCA | |
| | CTTCGTGGCCGAGGAGCAGGACTGACACGTGCTA | |
| | CGAGATTTCGATTCCACCGCCGCCTTCTATGAAA | |
| | GGTTGGGCTTCGGAATCGTTTTCCGGGACGCCGG | |
| | CTGGATGATCCTCCAGCGCGGGGATCTCATGCTG | |
| | GAGTTCTTCGCCCACCCCAACTTGTTTATTGCAGC | |
| | TTATAATGGTTACAAATAAAGCAATAGCATCACA | |
| | AATTTCACAAATAAAGCATTTTTTTCACTGCATTC | |
| | TAGTTGTGGTTTGTCCAAACTCATCAATGTATCTT | |
| | ATCATGTCTGGCGGCCGCTCTTGAAAGGAGTGGG | |
| | AATTGGCTCCGGTGCCCGTCAGTGGGCAGAGCGC | |
| | ACATCGCCCACAGTCCCCGAGAAGTTGGGGGGA | |
| | GGGGTCGGCAATTGAACCGGTGCCTAGAGAAGG | |
| | TGGCGCGGGGTAAACTGGGAAAGTGATGTCGTGT | |
| | ACTGGCTCCGCCTTTTTCCCGAGGGTGGGGGAGA | |
| | ACCGTATATAAGTGCAGTAGTCGCCGTGAACGTT | |
| | CTTTTTCGCAACGGGTTTGCCGCCAGAACACAGG | |
| | TAAGTGCCGTGTGTGGTTCCCGCGGGCCTGGCCT | |
| | CTTTACGGGTTATGGCCCTTGCGTGCCTTGAATTA | |
| | CTTCCACCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGC | |
| | TTGAGTTGAGGCCTGGCCTGGGCGCTGGGGCCGC | |
| | CGCGTGCGAATCTGGTGGCACCTTCGCGCCTGTC | |
| | TCGCTGCTTTCGATAAGTCTCTAGCCATTTAAAAT | |
| | TTTTGATGACCTGCTGCGACGCTTTTTTTCTGGCA | |
| | AGATAGTCTTGTAAATGCGGGCCAAGATCTGCAC | |
| | ACTGGTATTTCGGTTTTTGGGGCCGCGGGCGGCG | |
| | ACGGGGCCCGTGCGTCCCAGCGCACATGTTCGGC | |
| | GAGGCGGGGCCTGCGAGCGCGGCCACCGAGAAT | |
| | CGGACGGGGGTAGTCTCAAGCTGGCCGGCCTGCT | |
| | CTGGTGCCTGGCCTCGCGCCGCCGTGTATCGCCC | |
| | CGCCCTGGGCGGCAAGGCTGGCCCGGTCGGCACC | |
| | AGTTGCGTGAGCGGAAAGATGGCCGCTTCCCGGC | |
| | CCTGCTGCAGGGAGCTCAAAATGGAGGACGCGG | |
| | CGCTCGGGAGAGCGGGCGGGTGAGTCACCCACA | |
| | CAAAGGAAAAGGGCCTTTCCGTCCTCAGCCGTCG | |
| | CTTCATGTGACTCCACGGAGTACCGGGCGCCGTC | |
| | CAGGCACCTCGATTAGTTCTCGAGCTTTTGGAGT | |
| | ACGTCGTCTTTAGGTTGGGGGGAGGGGTTTTATG | |
| | CGATGGAGTTTCCCCACACTGAGTGGGTGGAGAC | |
| | TGAAGTTAGGCCAGCTTGGCACTTGATGTAATTC | |
| | TCCTTGGAATTTGCCCTTTTTGAGTTTGGATCTTG | |
| | GTTCATTCTCAAGCCTCAGACAGTGGTTCAAAGT | |
| | TTTTTTCTTCCATTTCAGGTGTCGTGAGGAATTAG | |
| | CTAGCTTCGAGGGGCTCGCATCTCTCCTTCACGC | |
| | GCCCGCCGCCCTACCTGAGGCCGCCATCCACGCC | |
| | GGTTGAGTCGCGTTCTGCCGCCTCCCGCCTGTGGT | |
| | GCCTCCTGAACTGCGTCCGCCGTCTAGGTAAGTT | |
| | TAAAGCTCAGGTCGAGACCGGGCCTTTGTCCGGC | |
| | GCTCCCTTGGAGCCTACCTAGACTCAGCCGGCTC | |
| | TCCACGCTTTGCCTGACCCTGCTTGCTCAACTCTA | |
| | CGTCTTTGTTTCGTTTTCTGTTCTGCGCCGTTACA | |
| | GATCCAAGCTGTGACCGGCGCCTACGGTACCCCG | |
| | GGTCCGCTCAGCCACTGGCACGCATGTGAGAG | |
| | CGCCACTCTTTTTATCAAGAGTCTGCTGAAAT | |
| | GAATTCTAAGGGCTTTCAGATTTTATCGCTTCG | |
| | ATCACACTGTTTGTGAAGAAATCTAAACCTGG | |
| | AAAGCTGAATTAAGCCAGACATTCCAGTTGGC | |
| | TCACTCATGGTCCCTGGAAATCACGAAGGAAA | |
| | CAGGGCAGAGAGCTACCAGGAGTAGTAAACG | |
| | CTAACACACAGGCTTCTAATTCATCATGGCTTA | |
| | ACCAAAAGGCATGTTTCTCCATGGCCCGTTAG | |
| | CTGTCTGTTAGTTTGAAGGTAATGTGATGATA | |
| | GCCAGGCTGGAGCTACCTGGGTCCTGTTCCAG | |
| | GGACTCTTAGGGCAGTTACCTACATGACCCTT | |
| | CTGGACCTCAGCTGCCTGATCTGAGAATGGGG | |
| | AGTAACAGCATCTACTTACCAGAGCTGGGGAA | |
| | ATTAGAGACTGCACTGTGAAGGGTTCCAACCA | |
| | CTCCTGGCCCAGAGTAAGCACTAGGTGAGTTT | |
| | TATATAGAAATTTCTTCTGCACCATGAGCTCAT | |
| | GTATATGGCTTTCCAGGTCTGGACTTTTACAA | |
| | CTCAGACAGTTCATATCAATGTCACAAAGCTT | |
| | CTTAGTGCTGCAAAATATCTGGTAATCACCAA | |
| | GTGGAGAGAATGCTGTATTCAACAAGGCCTGA | |
| | TAACCAAGGACTCAGCCGACTTACTGGGGGTC | |
| | CTTTCCCAGCTGCAAGGTTCCTTGAGCCAGGG | |
| | GCAAGCTAGGCTGTGTTTCCAAGCATCTTCTT | |
| | CCTAAGGACTTTAGGTTCCTTGGCCACTTCCTT | |
| | TTATTTCAGTGAAAGCAGAGCCACTCCATTCA | |
| | CACTTGTGGTCACAGTAGTACAATTACTGCCA | |
| | ATTCTTCCCTCTTTCCCCCCCCCCCCCTACTCC | |
| | CTGCGCAGCTTCATTTGCTCCCAGGAGTGGCT | |
| | AGAAAAATACCAAATTCCAACCCACAGTTTCT | |
| | CTCTTCCACTTCCTGGAAACACCCGAGGGCTC | |
| | TGCACTTACTCAGCGGATTCAACTTCCACTTTC | |
| | CATCACTTATCCAGGGTGATGCTACTCCTTGG | |
| | CACCAAGCACCCTGCCTGACTCCACCCCCCTG | |
| | GCTTACAATAAAAGGCTGCCTCAGAGCAGCCA | |
| | GAAGTGCACATGGATATGGATCGCCTGCAGACCG | |
| | CGCTGCTGGTGGTGCTGGTGCTGCTGGCGGTGGCG | |
| | CTGCAGGCGACCGAAGCGGGCCCGTATGGCGCGAA | |
| | CATGGAAGATAGCGTGTGCTGCCGCGATTATGTGCG | |
| | CTATCGCCTGCCGCTGCGCGTGGTGAAACATTTTTAT | |
| | TGGACCAGCGATAGCTGCCCGCGCCCGGGCGTGGT | |
| | GCTGCTGACCTTTCGCGATAAAGAAATTTGCGCGGA | |
| | TCCGCGCGTGCCGTGGGTGAAAATGATTCTGAACAA | |
| | ACTGAGCCAGTAAGAATTCGTCGACTGTACAACG | |
| | CGTGTTTAAACTTCGAATCTAGATACGTACATAT | |
| | GATTTAAATGATCTAACTAACTAAACTTGTTTATT | |
| | GCACTTATAATGGTTACAAATAAAGCAATAGCAT | |
| | CACAAATTTCACAAATAAAGCATTTTTTTCACTGC | |
| | ATTCTAGTTGTGGTTTGTCCAAACTCATCAATGTA | |
| | TCTTATCATGTCTGGAATGACTCAAATGATGTCA | |
| | ATTAGTCTATCAGAAGCTCATCTGGTCTCCCTTCC | |
| | GGGGGACAAGACATCCCTGTTTAATATTTAAACA | |
| | GCAGTGTTCCCAAACTGGGTTCTTATATCCCTTGC | |
| | TCTGGTCAACCAGGTTGCAGGGTTTCCTGTCCTCA | |
| | CAGGAACGAAGTCCCTAAAGAAACAGTGGCAGC | |
| | CAGGTTTAGCCCCGGAATTGACTGGATTCCTTTTT | |
| | TAGGGCCCATTGGTATGGCTTTTTCCCCGTATCCC | |
| | CCCAGGTGTCTGCAGGCTCAAAGAGCAGCGAGA | |
| | AGCGTTCAGAGGAAAGCGATCCCGTGCCACCTTC | |
| | CCCGTGCCCGGGCTGTCCCCGCACGCTGCCGGCT | |
| | CGGGGATGCGGGGGGAGCGCCGGACCGGAGCGG | |
| | AGCCCCGGGCGGCTCGCTGCTGCCCCCTAGCGGG | |
| | GGAGGGACGTAATTACATCCCTGGGGGCTTTGGG | |
| | GGGGGGCTGTCCCTGATATCTATAACAAGAAAAT | |
| | ATATATATAATAAGTTATCACGTAAGTAGAACAT | |
| | GAAATAACAATATAATTATCGTATGAGTTAAATC | |
| | TTAAAAGTCACGTAAAAGATAATCATGCGTCATT | |
| | TTGACTCACGCGGTCGTTATAGTTCAAAATCAGT | |
| | GACACTTACCGCATTGACAAGCACGCCTCACGGG | |
| | AGCTCCAAGCGGCGACTGAGATGTCCTAAATGCA | |
| | CAGCGACGGATTCGCGCTATTTAGAAAGAGAGA | |
| | GCAATATTTCAAGAATGCATGCGTCAATTTTACG | |
| | CAGACTATCTTTCTAGGGTTAATCTAGCTGCATCA | |
| | GGATCATATCGTCGGGTCTTTTTTCCGGCTCAGTC | |
| | ATCGCCCAAGCTGGCGCTATCTGGGCATCGGGGA | |
| | GGAAGAAGCCCGTGCCTTTTCCCGCGAGGTTGAA | |
| | GCGGCATGGAAAGAGTTTGCCGAGGATGACTGCT | |
| | GCTGCATTGACGTTGAGCGAAAACGCACGTTTAC | |
| | CATGATGATTCGGGAAGGTGTGGCCATGCACGCC | |
| | TTTAACGGTGAACTGTTCGTTCAGGCCACCTGGG | |
| | ATACCAGTTCGTCGCGGCTTTTCCGGACACAGTT | |
| | CCGGATGGTCAGCCCGAAGCGCATCAGCAACCCG | |
| | AACAATACCGGCGACAGCCGGAACTGCCGTGCC | |
| | GGTGTGCAGATTAATGACAGCGGTGCGGCGCTGG | |
| | GATATTACGTCAGCGAGGACGGGTATCCTGGCTG | |
| | GATGCCGCAGAAATGGACATGGATACCCCGTGA | |
| | GTTACCCGGCGGGCGCGCTTGGCGTAATCATGGT | |
| | CATAGCTGTTTCCTGTGTGAAATTGTTATCCGCTC | |
| | ACAATTCCACACAACATACGAGCCGGAAGCATA | |
| | AAGTGTAAAGCCTGGGGTGCCTAATGAGTGAGCT | |
| | AACTCACATTAATTGCGTTGCGCTCACTGCCCGCT | |
| | TTCCAGTCGGGAAACCTGTCGTGCCAGCTGCATT | |
| | AATGAATCGGCCAACGCGCGGGGAGAGGCGGTT | |
| | TGCGTATTGGGCGCTCTTCCGCTTCCTCGCTCACT | |
| | GACTCGCTGCGCTCGGTCGTTCGGCTGCGGCGAG | |
| | CGGTATCAGCTCACTCAAAGGCGGTAATACGGTT | |
| | ATCCACAGAATCAGGGGATAACGCAGGAAAGAA | |
| | CATGTGAGCAAAAGGCCAGCAAAAGGCCAGGAA | |
| | CCGTAAAAAGGCCGCGTTGCTGGCGTTTTTCCAT | |
| | AGGCTCCGCCCCCCTGACGAGCATCACAAAAATC | |
| | GACGCTCAAGTCAGAGGTGGCGAAACCCGACAG | |
| | GACTATAAAGATACCAGGCGTTTCCCCCTGGAAG | |
| | CTCCCTCGTGCGCTCTCCTGTTCCGACCCTGCCGC | |
| | TTACCGGATACCTGTCCGCCTTTCTCCCTTCGGGA | |
| | AGCGTGGCGCTTTCTCATAGCTCACGCTGTAGGT | |
| | ATCTCAGTTCGGTGTAGGTCGTTCGCTCCAAGCT | |
| | GGGCTGTGTGCACGAACCCCCCGTTCAGCCCGAC | |
| | CGCTGCGCCTTATCCGGTAACTATCGTCTTGAGTC | |
| | CAACCCGGTAAGACACGACTTATCGCCACTGGCA | |
| | GCAGCCACTGGTAACAGGATTAGCAGAGCGAGG | |
| | TATGTAGGCGGTGCTACAGAGTTCTTGAAGTGGT | |
| | GGCCTAACTACGGCTACACTAGAAGGACAGTATT | |
| | TGGTATCTGCGCTCTGCTGAAGCCAGTTACCTTCG | |
| | GAAAAAGAGTTGGTAGCTCTTGATCCGGCAAACA | |
| | AACCACCGCTGGTAGCGGTGGTTTTTTTGTTTGCA | |
| | AGCAGCAGATTACGCGCAGAAAAAAAGGATCTC | |
| | AAGAAGATCCTTTGATCTTTTCTACGGGGTCTGA | |
| | CGCTCAGTGGAACGAAAACTCACGTTAAGGGATT | |
| | TTGGTCATGAGATTATCAAAAAGGATCTTCACCT | |
| | AGATCCTTTTAAATTAAAAATGAAGTTTTAAATC | |
| | AATCTAAAGTATATATGAGTAAACTTGGTCTGAC | |
| | AGTTACCAATGCTTAATCAGTGAGGCACCTATCT | |
| | CAGCGATCTGTCTATTTCGTTCATCCATAGTTGCC | |
| | TGACTCCCCGTCGTGTAGATAACTACGATACGGG | |
| | AGGGCTTACCATCTGGCCCCAGTGCTGCAATGAT | |
| | ACCGCGAGACCCACGCTCACCGGCTCCAGATTTA | |
| | TCAGCAATAAACCAGCCAGCCGGAAGGGCCGAG | |
| | CGCAGAAGTGGTCCTGCAACTTTATCCGCCTCCA | |
| | TCCAGTCTATTAATTGTTGCCGGGAAGCTAGAGT | |
| | AAGTAGTTCGCCAGTTAATAGTTTGCGCAACGTT | |
| | GTTGCCATTGCTACAGGCATCGTGGTGTCACGCT | |
| | CGTCGTTTGGTATGGCTTCATTCAGCTCCGGTTCC | |
| | CAACGATCAAGGCGAGTTACATGATCCCCCATGT | |
| | TGTGCAAAAAAGCGGTTAGCTCCTTCGGTCCTCC | |
| | GATCGTTGTCAGAAGTAAGTTGGCCGCAGTGTTA | |
| | TCACTCATGGTTATGGCAGCACTGCATAATTCTCT | |
| | TACTGTCATGCCATCCGTAAGATGCTTTTCTGTGA | |
| | CTGGTGAGTGTACCAGCCAGGACAGAAATGCCTC | |
| | GACTTCGCTGCTACCCAAGGTTGCCGGGTGACGC | |
| | ACACCGTGGAAACGGATGAAGGCACGAACCCAG | |
| | TGGACATAAGCCTGTTCGGTTCGTAAGCTGTAAT | |
| | GCAAGTAGCGTATGCGCTCACGCAACTGGTCCAG | |
| | AACCTTGACCGAACGCAGCGGTGGTAACGGCGC | |
| | AGTGGCGGTTTTCATGGCTTGTTATGACTGTTTTT | |
| | TTGGGGTACAGTCTATGCCTCGGGCATCCAAGCA | |
| | GCAAGCGCGTTACGCCGTGGGTCGATGTTTGATG | |
| | TTATGGAGCAGCAACGATGTTACGCAGCAGGGCA | |
| | GTCGCCCTAAAACAAAGTTAAACATTATGAGGGA | |
| | AGCGGTGATCGCCGAAGTATCGACTCAACTATCA | |
| | GAGGTAGTTGGCGTCATCGAGCGCCATCTCGAAC | |
| | CGACGTTGCTGGCCGTACATTTGTACGGCTCCGC | |
| | AGTGGATGGCGGCCTGAAGCCACACAGTGATATT | |
| | GATTTGCTGGTTACGGTGACCGTAAGGCTTGATG | |
| | AAACAACGCGGCGAGCTTTGATCAACGACCTTTT | |
| | GGAAACTTCGGCTTCCCCTGGAGAGAGCGAGATT | |
| | CTCCGCGCTGTAGAAGTCACCATTGTTGTGCACG | |
| | ACGACATCATTCCGTGGCGTTATCCAGCTAAGCG | |
| | CGAACTGCAATTTGGAGAATGGCAGCGCAATGAC | |
| | ATTCTTGCAGGTATCTTCGAGCCAGCCACGATCG | |
| | ACATTGATCTGGCTATCTTGCTGACAAAAGCAAG | |
| | AGAACATAGCGTTGCCTTGGTAGGTCCAGCGGCG | |
| | GAGGAACTCTTTGATCCGGTTCCTGAACAGGATC | |
| | TATTTGAGGCGCTAAATGAAACCTTAACGCTATG | |
| | GAACTCGCCGCCCGACTGGGCTGGCGATGAGCGA | |
| | AATGTAGTGCTTACGTTGTCCCGCATTTGGTACA | |
| | GCGCAGTAACCGGCAAAATCGCGCCGAAGGATG | |
| | TCGCTGCCGACTGGGCAATGGAGCGCCTGCCGGC | |
| | CCAGTATCAGCCCGTCATACTTGAAGCTAGACAG | |
| | GCTTATCTTGGACAAGAAGAAGATCGCTTGGCCT | |
| | CGCGCGCAGATCAGTTGGAAGAATTTGTCCACTA | |
| | CGTGAAAGGCGAGATCACCAAGGTAGTCGGCAA | |
| | ATAACCCTCGAGCCACCCATGACCAAAATCCCTT | |
| | AACGTGAGTTA | |
| 3 | TTAATTAAGCTAGCTCAGACCGTTAGACGCCAGG | BRE-ActRIIAFc |
| | ACGGGCTGTCAGGCTGGCGCCGCGGCGCCAGCCT | construct |
| | GACAGCCCGTCCTGGCGTCTAACGGTCTGAGCTA | |
| | GCCCGGGCTCGAGATCTGGGCTATAAAAGGGGGT | |
| | GGGGGCGCGTTCGTCCTCACTCTCTTCCAAGCTTG | |
| | GCATTCCGGTACTGTTGGTAAAGCCACCATGGGC | |
| | GCGGCGGCGAAACTGGCGTTTGCGGTGTTTCTGA | |
| | TTAGCTGCAGCAGCGGCGCGATTCTGGGCCGCAG | |
| | CGAAACCCAGGAATGCCTGTTTTTTAACGCGAAC | |
| | TGGGAAAAAGATCGCACCAACCAGACCGGCGTG | |
| | GAACCGTGCTATGGCGATAAAGATAAACGCCGCC | |
| | ATTGCTTTGCGACCTGGAAAAACATTAGCGGCAG | |
| | CATTGAAATTGTGAAACAGGGCTGCTGGCTGGAT | |
| | GATATTAACTGCTATGATCGCACCGATTGCGTGG | |
| | AAAAAAAAGATAGCCCGGAAGTGTATTTTTGCTG | |
| | CTGCGAAGGCAACATGTGCAACGAAAAATTTAGC | |
| | TATTTTCCGGAAATGGAAGTGACCCAGCCGACCA | |
| | GCAACCCGGTGACCCCGAAACCGCCGATTGAAG | |
| | GCCGCATGGATCCGAAAAGCTGCGATAAAACCC | |
| | ATACCTGCCCGCCGTGCCCGGCGCCGGAACTGCT | |
| | GGGCGGCCCGAGCGTGTTTCTGTTTCCGCCGAAA | |
| | CCGAAAGATACCCTGATGATTAGCCGCACCCCGG | |
| | AAGTGACCTGCGTGGTGGTGGATGTGAGCCATGA | |
| | AGATCCGGAAGTGAAATTTAACTGGTATGTGGAT | |
| | GGCGTGGAAGTGCATAACGCGAAAACCAAACCG | |
| | CGCGAAGAACAGTATAACAGCACCTATCGCGTGG | |
| | TGAGCGTGCTGACCGTGCTGCATCAGGATTGGCT | |
| | GAACGGCAAAGAATATAAATGCAAAGTGAGCAA | |
| | CAAAGCGCTGCCGGCGCCGATTGAAAAAACCATT | |
| | AGCAAAGCGAAAGGCCAGCCGCGCGAACCGCAG | |
| | GTGTATACCCTGCCGCCGAGCCGCGATGAACTGA | |
| | CCAAAAACCAGGTGAGCCTGACCTGCCTGGTGAA | |
| | AGGCTTTTATCCGAGCGATATTGCGGTGGAATGG | |
| | GAAAGCAACGGCCAGCCGGAAAACAACTATAAA | |
| | ACCACCCCGCCGGTGCTGGATAGCGATGGCAGCT | |
| | TTTTTCTGTATAGCAAACTGACCGTGGATAAAAG | |
| | CCGCTGGCAGCAGGGCAACGTGTTTAGCTGCAGC | |
| | GTGATGCATGAAGCGCTGCATAACCATTATACCC | |
| | AGAAAAGCCTGAGCCTGAGCCCGGGCAAAG | |
| 4 | CAGTGGCAGCCAGGTTTAGCCCCGGAATTGACTG | BRE-ActRIIAFc |
| | GATTCCTTTTTTAGGGCCCATTGGTATGGCTTTTT | transposon vector |
| | CCCCGTATCCCCCCAGGTGTCTGCAGGCTCAAAG | (BRE-ActRIIAFc |
| | AGCAGCGAGAAGCGTTCAGAGGAAAGCGATCCC | construct underlined, |
| | GTGCCACCTTCCCCGTGCCCGGGCTGTCCCCGCA | BRE promoter bold, |
| | CGCTGCCGGCTCGGGGATGCGGGGGGAGCGCCG | ActRIIAFc gene |
| | GACCGGAGCGGAGCCCCGGGCGGCTCGCTGCTGC | italicized) |
| | CCCCTAGCGGGGGAGGGACGTAATTACATCCCTG | |
| | GGGGCTTTGGGGGGGGGCTGTCCCTGATATCTAT | |
| | AACAAGAAAATATATATATAATAAGTTATCACGT | |
| | AAGTAGAACATGAAATAACAATATAATTATCGTA | |
| | TGAGTTAAATCTTAAAAGTCACGTAAAAGATAAT | |
| | CATGCGTCATTTTGACTCACGCGGTCGTTATAGTT | |
| | CAAAATCAGTGACACTTACCGCATTGACAAGCAC | |
| | GCCTCACGGGAGCTCCAAGCGGCGACTGAGATGT | |
| | CCTAAATGCACAGCGACGGATTCGCGCTATTTAG | |
| | AAAGAGAGAGCAATATTTCAAGAATGCATGCGTC | |
| | AATTTTACGCAGACTATCTTTCTAGGGTTAATCTA | |
| | GCTGCATCAGGATCATATCGTCGGGTCTTTTTTCC | |
| | GGCTCAGTCATCGCCCAAGCTGGCGCTATCTGGG | |
| | CATCGGGGAGGAAGAAGCCCGTGCCTTTTCCCGC | |
| | GAGGTTGAAGCGGCATGGAAAGAGTTTGCCGAG | |
| | GATGACTGCTGCTGCATTGACGTTGAGCGAAAAC | |
| | GCACGTTTACCATGATGATTCGGGAAGGTGTGGC | |
| | CATGCACGCCTTTAACGGTGAACTGTTCGTTCAG | |
| | GCCACCTGGGATACCAGTTCGTCGCGGCTTTTCC | |
| | GGACACAGTTCCGGATGGTCAGCCCGAAGCGCAT | |
| | CAGCAACCCGAACAATACCGGCGACAGCCGGAA | |
| | CTGCCGTGCCGGTGTGCAGATTAATGACAGCGGT | |
| | GCGGCGCTGGGATATTACGTCAGCGAGGACGGGT | |
| | ATCCTGGCTGGATGCCGCAGAAATGGACATGGAT | |
| | ACCCCGTGAGTTACCCGGCGGGCGCGCTTGGCGT | |
| | AATCATGGTCATAGCTGTTTCCTGTGTGAAATTGT | |
| | TATCCGCTCACAATTCCACACAACATACGAGCCG | |
| | GAAGCATAAAGTGTAAAGCCTGGGGTGCCTAATG | |
| | AGTGAGCTAACTCACATTAATTGCGTTGCGCTCA | |
| | CTGCCCGCTTTCCAGTCGGGAAACCTGTCGTGCC | |
| | AGCTGCATTAATGAATCGGCCAACGCGCGGGGA | |
| | GAGGCGGTTTGCGTATTGGGCGCTCTTCCGCTTCC | |
| | TCGCTCACTGACTCGCTGCGCTCGGTCGTTCGGCT | |
| | GCGGCGAGCGGTATCAGCTCACTCAAAGGCGGTA | |
| | ATACGGTTATCCACAGAATCAGGGGATAACGCAG | |
| | GAAAGAACATGTGAGCAAAAGGCCAGCAAAAGG | |
| | CCAGGAACCGTAAAAAGGCCGCGTTGCTGGCGTT | |
| | TTTCCATAGGCTCCGCCCCCCTGACGAGCATCAC | |
| | AAAAATCGACGCTCAAGTCAGAGGTGGCGAAAC | |
| | CCGACAGGACTATAAAGATACCAGGCGTTTCCCC | |
| | CTGGAAGCTCCCTCGTGCGCTCTCCTGTTCCGACC | |
| | CTGCCGCTTACCGGATACCTGTCCGCCTTTCTCCC | |
| | TTCGGGAAGCGTGGCGCTTTCTCATAGCTCACGC | |
| | TGTAGGTATCTCAGTTCGGTGTAGGTCGTTCGCTC | |
| | CAAGCTGGGCTGTGTGCACGAACCCCCCGTTCAG | |
| | CCCGACCGCTGCGCCTTATCCGGTAACTATCGTCT | |
| | TGAGTCCAACCCGGTAAGACACGACTTATCGCCA | |
| | CTGGCAGCAGCCACTGGTAACAGGATTAGCAGA | |
| | GCGAGGTATGTAGGCGGTGCTACAGAGTTCTTGA | |
| | AGTGGTGGCCTAACTACGGCTACACTAGAAGGAC | |
| | AGTATTTGGTATCTGCGCTCTGCTGAAGCCAGTT | |
| | ACCTTCGGAAAAAGAGTTGGTAGCTCTTGATCCG | |
| | GCAAACAAACCACCGCTGGTAGCGGTGGTTTTTT | |
| | TGTTTGCAAGCAGCAGATTACGCGCAGAAAAAA | |
| | AGGATCTCAAGAAGATCCTTTGATCTTTTCTACG | |
| | GGGTCTGACGCTCAGTGGAACGAAAACTCACGTT | |
| | AAGGGATTTTGGTCATGAGATTATCAAAAAGGAT | |
| | CTTCACCTAGATCCTTTTAAATTAAAAATGAAGTT | |
| | TTAAATCAATCTAAAGTATATATGAGTAAACTTG | |
| | GTCTGACAGTTACCAATGCTTAATCAGTGAGGCA | |
| | CCTATCTCAGCGATCTGTCTATTTCGTTCATCCAT | |
| | AGTTGCCTGACTCCCCGTCGTGTAGATAACTACG | |
| | ATACGGGAGGGCTTACCATCTGGCCCCAGTGCTG | |
| | CAATGATACCGCGAGACCCACGCTCACCGGCTCC | |
| | AGATTTATCAGCAATAAACCAGCCAGCCGGAAG | |
| | GGCCGAGCGCAGAAGTGGTCCTGCAACTTTATCC | |
| | GCCTCCATCCAGTCTATTAATTGTTGCCGGGAAG | |
| | CTAGAGTAAGTAGTTCGCCAGTTAATAGTTTGCG | |
| | CAACGTTGTTGCCATTGCTACAGGCATCGTGGTG | |
| | TCACGCTCGTCGTTTGGTATGGCTTCATTCAGCTC | |
| | CGGTTCCCAACGATCAAGGCGAGTTACATGATCC | |
| | CCCATGTTGTGCAAAAAAGCGGTTAGCTCCTTCG | |
| | GTCCTCCGATCGTTGTCAGAAGTAAGTTGGCCGC | |
| | AGTGTTATCACTCATGGTTATGGCAGCACTGCAT | |
| | AATTCTCTTACTGTCATGCCATCCGTAAGATGCTT | |
| | TTCTGTGACTGGTGAGTACTCAACCAAGTCATTCT | |
| | GAGAATAGTGTATGCGGCGACCGAGTTGCTCTTG | |
| | CCCGGCGTCAATACGGGATAATACCGCGCCACAT | |
| | AGCAGAACTTTAAAAGTGCTCATCATTGGAAAAC | |
| | GTTCTTCGGGGCGAAAACTCTCAAGGATCTTACC | |
| | GCTGTTGAGATCCAGTTCGATGTAACCCACTCGT | |
| | GCACCCAACTGATCTTCAGCATCTTTTACTTTCAC | |
| | CAGCGTTTCTGGGTGAGCAAAAACAGGAAGGCA | |
| | AAATGCCGCAAAAAAGGGAATAAGGGCGACACG | |
| | GAAATGTTGAATACTCATACTCTTCCTTTTTCAAT | |
| | ATTATTGAAGCATTTATCAGGGTTATTGTCTCATG | |
| | AGCGGATACATATTTGAATGTATTTAGAAAAATA | |
| | AACAAATAGGGGTTCCGCGCACATTTCCCCGAAA | |
| | AGTGCCACCTAAATTGTAAGCGTTAATATTTTGTT | |
| | AAAATTCGCGTTAAATTTTTGTTAAATCAGCTCAT | |
| | TTTTTAACCAATAGGCCGAAATCGGCAAAATCCC | |
| | TTATAAATCAAAAGAATAGACCGAGATAGGGTTG | |
| | AGTGTTGTTCCAGTTTGGAACAAGAGTCCACTAT | |
| | TAAAGAACGTGGACTCCAACGTCAAAGGGCGAA | |
| | AAACCGTCTATCAGGGCGATGGCCCACTACGTGA | |
| | ACCATCACCCTAATCAAGTTTTTTGGGGTCGAGG | |
| | TGCCGTAAAGCACTAAATCGGAACCCTAAAGGG | |
| | AGCCCCCGATTTAGAGCTTGACGGGGAAAGCCGG | |
| | CGAACGTGGCGAGAAAGGAAGGGAAGAAAGCGA | |
| | AAGGAGCGGGCGCTAGGGCGCTGGCAAGTGTAG | |
| | CGGTCACGCTGCGCGTAACCACCACACCCGCCGC | |
| | GCTTAATGCGCCGCTACAGGGCGCGTCCCATTCG | |
| | CCATTCAGGCTGCGCAACTGTTGGGAAGGGCGAT | |
| | CGGTGCGGGCCTCTTCGCTATTACGCCAGCTGGC | |
| | GAAAGGGGGATGTGCTGCAAGGCGATTAAGTTG | |
| | GGTAACGCCAGGGTTTTCCCAGTCACGACGTTGT | |
| | AAAACGACGGCCAGTGAGCGCGCCTCGTTCATTC | |
| | ACGTTTTTGAACCCGTGGAGGACGGGCAGACTCG | |
| | CGGTGCAAATGTGTTTTACAGCGTGATGGAGCAG | |
| | ATGAAGATGCTCGACACGCTGCAGAACACGCAG | |
| | CTAGATTAACCCTAGAAAGATAATCATATTGTGA | |
| | CGTACGTTAAAGATAATCATGTGTAAAATTGACG | |
| | CATGTGTTTTATCGGTCTGTATATCGAGGTTTATT | |
| | TATTAATTTGAATAGATATTAAGTTTTATTATATT | |
| | TACACTTACATACTAATAATAAATTCAACAAACA | |
| | ATTTATTTATGTTTATTTATTTATTAAAAAAAACA | |
| | AAAACTCAAAATTTCTTCTATAAAGTAACAAAAC | |
| | TTTTATGAGGGACAGCCCCCCCCCAAAGCCCCCA | |
| | GGGATGTAATTACGTCCCTCCCCCGCTAGGGGGC | |
| | AGCAGCGAGCCGCCCGGGGCTCCGCTCCGGTCCG | |
| | GCGCTCCCCCCGCATCCCCGAGCCGGCAGCGTGC | |
| | GGGGACAGCCCGGGCACGGGGAAGGTGGCACGG | |
| | GATCGCTTTCCTCTGAACGCTTCTCGCTGCTCTTT | |
| | GAGCCTGCAGACACCTGGGGGGATACGGGGAAA | |
| | AGGCCTCCACGGCCTTGCGTTTAAACAGATCTAC | |
| | GCGTTTAATTAAGCTAGCTCAGACCGTTAGAC | |
| | GCCAGGACGGGCTGTCAGGCTGGCGCCGCGG | |
| | CGCCAGCCTGACAGCCCGTCCTGGCGTCTAAC | |
| | GGTCTGAGCTAGCCCGGGCTCGAGATCTGGGC | |
| | TATAAAAGGGGGTGGGGGCGCGTTCGTCCTCA | |
| | CTCTCTTCCAAGCTTGGCATTCCGGTACTGTT | |
| | GGTAAAGCCACCATGGGCGCGGCGGCGAAACTG | |
| | GCGTTTGCGGTGTTTCTGATTAGCTGCAGCAGCGGC | |
| | GCGATTCTGGGCCGCAGCGAAACCCAGGAATGCCT | |
| | GTTTTTTAACGCGAACTGGGAAAAAGATCGCACCAAC | |
| | CAGACCGGCGTGGAACCGTGCTATGGCGATAAAGAT | |
| | AAACGCCGCCATTGCTTTGCGACCTGGAAAAACATTA | |
| | GCGGCAGCATTGAAATTGTGAAACAGGGCTGCTGGC | |
| | TGGATGATATTAACTGCTATGATCGCACCGATTGCGT | |
| | GGAAAAAAAAGATAGCCCGGAAGTGTATTTTTGCTGC | |
| | TGCGAAGGCAACATGTGCAACGAAAAATTTAGCTATT | |
| | TTCCGGAAATGGAAGTGACCCAGCCGACCAGCAACC | |
| | CGGTGACCCCGAAACCGCCGATTGAAGGCCGCATG | |
| | GATCCGAAAAGCTGCGATAAAACCCATACCTGCCCG | |
| | CCGTGCCCGGCGCCGGAACTGCTGGGCGGCCCGA | |
| | GCGTGTTTCTGTTTCCGCCGAAACCGAAAGATACCC | |
| | TGATGATTAGCCGCACCCCGGAAGTGACCTGCGTGG | |
| | TGGTGGATGTGAGCCATGAAGATCCGGAAGTGAAAT | |
| | TTAACTGGTATGTGGATGGCGTGGAAGTGCATAACG | |
| | CGAAAACCAAACCGCGCGAAGAACAGTATAACAGCA | |
| | CCTATCGCGTGGTGAGCGTGCTGACCGTGCTGCATC | |
| | AGGATTGGCTGAACGGCAAAGAATATAAATGCAAAG | |
| | TGAGCAACAAAGCGCTGCCGGCGCCGATTGAAAAAA | |
| | CCATTAGCAAAGCGAAAGGCCAGCCGCGCGAACCG | |
| | CAGGTGTATACCCTGCCGCCGAGCCGCGATGAACT | |
| | GACCAAAAACCAGGTGAGCCTGACCTGCCTGGTGAA | |
| | AGGCTTTTATCCGAGCGATATTGCGGIGGAATGGGA | |
| | AAGCAACGGCCAGCCGGAAAACAACTATAAAACCAC | |
| | CCCGCCGGTGCTGGATAGCGATGGCAGCTTTTTTCT | |
| | GTATAGCAAACTGACCGTGGATAAAAGCCGCTGGCA | |
| | GCAGGGCAACGTGTTTAGCTGCAGCGTGATGCATGA | |
| | AGCGCTGCATAACCATTATACCCAGAAAAGCCTGAG | |
| | CCTGAGCCCGGGCAAAGCTACTAACTTCAGCCTGC | |
| | TGAAGCAGGCTGGCGACGTGGAGGAGAACCCTG | |
| | GACCTATGGAAGATGCCAAAAACATTAAGAAGG | |
| | GCCCAGCGCCATTCTACCCACTCGAAGACGGGAC | |
| | CGCCGGCGAGCAGCTGCACAAAGCCATGAAGCG | |
| | CTACGCCCTGGTGCCCGGCACCATCGCCTTTACC | |
| | GACGCACATATCGAGGTGGACATTACCTACGCCG | |
| | AGTACTTCGAGATGAGCGTTCGGCTGGCAGAAGC | |
| | TATGAAGCGCTATGGGCTGAATACAAACCATCGG | |
| | ATCGTGGTGTGCAGCGAGAATAGCTTGCAGTTCT | |
| | TCATGCCCGTGTTGGGTGCCCTGTTCATCGGTGTG | |
| | GCTGTGGCCCCAGCTAACGACATCTACAACGAGC | |
| | GCGAGCTGCTGAACAGCATGGGCATCAGCCAGCC | |
| | CACCGTCGTATTCGTGAGCAAGAAAGGGCTGCAA | |
| | AAGATCCTCAACGTGCAAAAGAAGCTACCGATCA | |
| | TACAAAAGATCATCATCATGGATAGCAAGACCGA | |
| | CTACCAGGGCTTCCAAAGCATGTACACCTTCGTG | |
| | ACTTCCCATTTGCCACCCGGCTTCAACGAGTACG | |
| | ACTTCGTGCCCGAGAGCTTCGACCGGGACAAAAC | |
| | CATCGCCCTGATCATGAACAGTAGTGGCAGTACC | |
| | GGATTGCCCAAGGGCGTAGCCCTACCGCACCGCA | |
| | CCGCTTGTGTCCGATTCAGTCATGCCCGCGACCC | |
| | CATCTTCGGCAACCAGATCATCCCCGACACCGCT | |
| | ATCCTCAGCGTGGTGCCATTTCACCACGGCTTCG | |
| | GCATGTTCACCACGCTGGGCTACTTGATCTGCGG | |
| | CTTTCGGGTCGTGCTCATGTACCGCTTCGAGGAG | |
| | GAGCTATTCTTGCGCAGCTTGCAAGACTATAAGA | |
| | TTCAATCTGCCCTGCTGGTGCCCACACTATTTAGC | |
| | TTCTTCGCTAAGAGCACTCTCATCGACAAGTACG | |
| | ACCTAAGCAACTTGCACGAGATCGCCAGCGGCGG | |
| | GGCGCCGCTCAGCAAGGAGGTAGGTGAGGCCGT | |
| | GGCCAAACGCTTCCACCTACCAGGCATCCGCCAG | |
| | GGCTACGGCCTGACAGAAACAACCAGCGCCATTC | |
| | TGATCACCCCCGAAGGGGACGACAAGCCTGGCG | |
| | CAGTAGGCAAGGTGGTGCCCTTCTTCGAGGCTAA | |
| | GGTGGTGGACTTGGACACCGGTAAGACACTGGGT | |
| | GTGAACCAGCGCGGCGAGCTGTGCGTCCGTGGCC | |
| | CCATGATCATGAGCGGCTACGTTAACAACCCCGA | |
| | GGCTACAAACGCTCTCATCGACAAGGACGGCTGG | |
| | CTGCACAGCGGCGACATCGCCTACTGGGACGAGG | |
| | ACGAGCACTTCTTCATCGTGGACCGGCTGAAGAG | |
| | CCTGATCAAATACAAGGGCTACCAGGTAGCCCCA | |
| | GCCGAACTGGAGAGCATCCTGCTGCAACACCCCA | |
| | ACATCTTCGACGCCGGGGTCGCCGGCCTGCCCGA | |
| | CGACGATGCCGGCGAGCTGCCCGCCGCAGTCGTC | |
| | GTGCTGGAACACGGTAAAACCATGACCGAGAAG | |
| | GAGATCGTGGACTATGTGGCCAGCCAGGTTACAA | |
| | CCGCCAAGAAGCTGCGCGGTGGTGTTGTGTTCGT | |
| | GGACGAGGTGCCTAAAGGACTGACCGGCAAGTT | |
| | GGACGCCCGCAAGATCCGCGAGATTCTCATTAAG | |
| | GCCAAGAAGGGCGGCAAGATCGCCGTGTAAATTT | |
| | AAATCGGCCTCGACTGTGCCTTCTAGTTGCCAGC | |
| | CATCTGTTGTTTGCCCCTCCCCCGTGCCTTCCTTG | |
| | ACCCTGGAAGGTGCCACTCCCACTGTCCTTTCCTA | |
| | ATAAAATGAGGAAATTGCATCGCATTGTCTGAGT | |
| | AGGTGTCATTCTATTCTGGGGGGTGGGGGGGGC | |
| | AGGACAGCAAGGGGGAGGATTGGGAAGACAATA | |
| | GCAGGCATGCTGGGGACGCGGCCGCAATTTAACG | |
| | CGAATTAATTCTGTGGAATGTGTGTCAGTTAGGG | |
| | TGTGGAAAGTCCCCAGGCTCCCCAGCAGGCAGAA | |
| | GTATGCAAAGCATGCATCTCAATTAGTCAGCAAC | |
| | CAGGTGTGGAAAGTCCCCAGGCTCCCCAGCAGGC | |
| | AGAAGTATGCAAAGCATGCATCTCAATTAGTCAG | |
| | CAACCATAGTCCCGCCCCTAACTCCGCCCATCCC | |
| | GCCCCTAACTCCGCCCAGTTCCGCCCATTCTCCGC | |
| | CCCATGGCTGACTAATTTTTTTTATTTATGCAGAG | |
| | GCCGAGGCCGCCTCTGCCTCTGAGCTATTCCAGA | |
| | AGTAGTGAGGAGGCTTTTTTGGAGGCCTAGGCTT | |
| | TTGCAAAAAGCTCCCGGGAGCTTGTATATCCATT | |
| | TTCGGATCTGATCAGCACGTGATGAAAAAGCCTG | |
| | AACTCACCGCGACGTCTGTCGAGAAGTTTCTGAT | |
| | CGAAAAGTTCGACAGCGTCTCCGACCTGATGCAG | |
| | CTCTCGGAGGGCGAAGAATCTCGTGCTTTCAGCT | |
| | TCGATGTAGGAGGGCGTGGATATGTCCTGCGGGT | |
| | AAATAGCTGCGCCGATGGTTTCTACAAAGATCGT | |
| | TATGTTTATCGGCACTTTGCATCGGCCGCGCTCCC | |
| | GATTCCGGAAGTGCTTGACATTGGGGAATTCAGC | |
| | GAGAGCCTGACCTATTGCATCTCCCGCCGTGCAC | |
| | AGGGTGTCACGTTGCAAGACCTGCCTGAAACCGA | |
| | ACTGCCCGCTGTTCTGCAGCCGGTCGCGGAGGCC | |
| | ATGGATGCGATCGCTGCGGCCGATCTTAGCCAGA | |
| | CGAGCGGGTTCGGCCCATTCGGACCGCAAGGAAT | |
| | CGGTCAATACACTACATGGCGTGATTTCATATGC | |
| | GCGATTGCTGATCCCCATGTGTATCACTGGCAAA | |
| | CTGTGATGGACGACACCGTCAGTGCGTCCGTCGC | |
| | GCAGGCTCTCGATGAGCTGATGCTTTGGGCCGAG | |
| | GACTGCCCCGAAGTCCGGCACCTCGTGCACGCGG | |
| | ATTTCGGCTCCAACAATGTCCTGACGGACAATGG | |
| | CCGCATAACAGCGGTCATTGACTGGAGCGAGGCG | |
| | ATGTTCGGGGATTCCCAATACGAGGTCGCCAACA | |
| | TCTTCTTCTGGAGGCCGTGGTTGGCTTGTATGGAG | |
| | CAGCAGACGCGCTACTTCGAGCGGAGGCATCCGG | |
| | AGCTTGCAGGATCGCCGCGGCTCCGGGCGTATAT | |
| | GCTCCGCATTGGTCTTGACCAACTCTATCAGAGC | |
| | TTGGTTGACGGCAATTTCGATGATGCAGCTTGGG | |
| | CGCAGGGTCGATGCGACGCAATCGTCCGATCCGG | |
| | AGCCGGGACTGTCGGGCGTACACAAATCGCCCGC | |
| | AGAAGCGCGGCCGTCTGGACCGATGGCTGTGTAG | |
| | AAGTACTCGCCGATAGTGGAAACCGACGCCCCAG | |
| | CACTCGTCCGAGGGCAAAGGAATAGGTCGACAA | |
| | TCAACCTCTGGATTACAAAATTTGTGAAAGATTG | |
| | ACTGGTATTCTTAACTATGTTGCTCCTTTTACGCT | |
| | ATGTGGATACGCTGCTTTAATGCCTTTGTATCATG | |
| | CGTTAACTAAACTTGTTTATTGCAGCTTATAATGG | |
| | TTACAAATAAAGCAATAGCATCACAAATTTCACA | |
| | AATAAAGCATTTTTTTCACTGCATTCTAGTTGTGG | |
| | TTTGTCCAAACTCATCAATGTATCTTATCATGTCT | |
| | GGAATTGACTCAAATGATGTCAATTAGTCTATCA | |
| | GAAGCTCATCTGGTCTCCCTTCCGGGGGACAAGA | |
| | CATCCCTGTTTAATATTTAAACAGCAGTGTTCCCA | |
| | AACTGGGTTCTTATATCCCTTGCTCTGGTCAACCA | |
| | GGTTGCAGGGTTTCCTGTCCTCACAGGAACGAAG | |
| | TCCCTAAAGAAA | |
| 5 | CCCGGGCTCGAGAGCCAGACAAAAAGCCAGACA | CAGA-TGFbRIIFc |
| | TTTAGCCAGACACTCGAGAGCCAGACAAAAAGC | construct |
| | CAGACATTTAGCCAGACACTCGAGAGCCAGACA | |
| | AAAAGCCAGACATTTAGCCAGACACTCGAGTGTC | |
| | TGGCTAAATGTCTGGCTTTTTGTCTGGCTCTCGAG | |
| | ATCTGGGCTATAAAAGGGGGTGGGGGCGCGTTCG | |
| | TCCTCACTCTCTTCCAAGCTTAGGCGTGTACGGTG | |
| | GGAGGCCTATATAAGCAGAGCTCGTTTAGTGAAC | |
| | CGTCAGATCGCACTAGTCCAAGTTTGTACAAAAA | |
| | AGCAGGCTGCCACCATGGGCCGCGGCCTGCTGCG | |
| | CGGCCTGTGGCCGCTGCATATTGTGCTGTGGACC | |
| | CGCATTGCGAGCACCATTCCGCCGCATGTGCAGA | |
| | AAAGCGTGAACAACGATATGATTGTGACCGATAA | |
| | CAACGGCGCGGTGAAATTTCCGCAGCTGTGCAAA | |
| | TTTTGCGATGTGCGCTTTAGCACCTGCGATAACC | |
| | AGAAAAGCTGCATGAGCAACTGCAGCATTACCA | |
| | GCATTTGCGAAAAACCGCAGGAAGTGTGCGTGGC | |
| | GGTGTGGCGCAAAAACGATGAAAACATTACCCTG | |
| | GAAACCGTGTGCCATGATCCGAAACTGCCGTATC | |
| | ATGATTTTATTCTGGAAGATGCGGCGAGCCCGAA | |
| | ATGCATTATGAAAGAAAAAAAAAAACCGGGCGA | |
| | AACCTTTTTTATGTGCAGCTGCAGCAGCGATGAA | |
| | TGCAACGATAACATTATTTTTAGCGAAGAATATA | |
| | ACACCAGCAACCCGGATATGGATCCGAAAAGCT | |
| | GCGATAAAACCCATACCTGCCCGCCGTGCCCGGC | |
| | GCCGGAACTGCTGGGCGGCCCGAGCGTGTTTCTG | |
| | TTTCCGCCGAAACCGAAAGATACCCTGATGATTA | |
| | GCCGCACCCCGGAAGTGACCTGCGTGGTGGTGGA | |
| | TGTGAGCCATGAAGATCCGGAAGTGAAATTTAAC | |
| | TGGTATGTGGATGGCGTGGAAGTGCATAACGCGA | |
| | AAACCAAACCGCGCGAAGAACAGTATAACAGCA | |
| | CCTATCGCGTGGTGAGCGTGCTGACCGTGCTGCA | |
| | TCAGGATTGGCTGAACGGCAAAGAATATAAATGC | |
| | AAAGTGAGCAACAAAGCGCTGCCGGCGCCGATT | |
| | GAAAAAACCATTAGCAAAGCGAAAGGCCAGCCG | |
| | CGCGAACCGCAGGTGTATACCCTGCCGCCGAGCC | |
| | GCGATGAACTGACCAAAAACCAGGTGAGCCTGA | |
| | CCTGCCTGGTGAAAGGCTTTTATCCGAGCGATAT | |
| | TGCGGTGGAATGGGAAAGCAACGGCCAGCCGGA | |
| | AAACAACTATAAAACCACCCCGCCGGTGCTGGAT | |
| | AGCGATGGCAGCTTTTTTCTGTATAGCAAACTGA | |
| | CCGTGGATAAAAGCCGCTGGCAGCAGGGCAACG | |
| | TGTTTAGCTGCAGCGTGATGCATGAAGCGCTGCA | |
| | TAACCATTATACCCAGAAAAGCCTGAGCCTGAGC | |
| | CCGGGCAAA | |
| 6 | TTAACCCTAGAAAGATAGTCTGCGTAAAATTGAC | CAGA-TGFbRIIFc |
| | GCATGCATTCTTGAAATATTGCTCTCTCTTTCTAA | transposon vector |
| | ATAGCGCGAATCCGTCGCTGTGCATTTAGGACAT | (CAGA-TGFbRIIFc |
| | CTCAGTCGCCGCTTGGAGCTCCCGTGAGGCGTGC | construct underlined, |
| | TTGTCAATGCGGTAAGTGTCACTGATTTTGAACT | CAGA promoter bold, |
| | ATAACGACCGCGTGAGTCAAAATGACGCATGATT | TGFbRIIFc gene |
| | ATCTTTTACGTGACTTTTAAGATTTAACTCATACG | italicized) |
| | ATAATTATATTGTTATTTCATGTTCTACTTACGTG | |
| | ATAACTTATTATATATATATTTTCTTGTTATAGAT | |
| | ATCATCAACTTTGTATAGAAAAGTTGCCCGGGCT | |
| | CGAGAGCCAGACAAAAAGCCAGACATTTAGCC | |
| | AGACACTCGAGAGCCAGACAAAAAGCCAGACA | |
| | TTTAGCCAGACACTCGAGAGCCAGACAAAAAG | |
| | CCAGACATTTAGCCAGACACTCGAGTGTCTGG | |
| | CTAAATGTCTGGCTTTTTGTCTGGCTCTCGAG | |
| | ATCTGGGCTATAAAAGGGGGTGGGGGCGCGT | |
| | TCGTCCTCACTCTCTTCCAAGCTTAGGCGTGT | |
| | ACGGTGGGAGGCCTATATAAGCAGAGCTCGTT | |
| | TAGTGAACCGTCAGATCGCACTAGTCCAAGTTT | |
| | GTACAAAAAAGCAGGCTGCCACCATGGGCCGCGG | |
| | CCTGCTGCGCGGCCTGTGGCCGCTGCATATTGTGCT | |
| | GTGGACCCGCATTGCGAGCACCATTCCGCCGCATGT | |
| | GCAGAAAAGCGTGAACAACGATATGATTGTGACCGA | |
| | TAACAACGGCGCGGTGAAATTTCCGCAGCTGTGCAA | |
| | ATTTTGCGATGTGCGCTTTAGCACCTGCGATAACCAG | |
| | AAAAGCTGCATGAGCAACTGCAGCATTACCAGCATTT | |
| | GCGAAAAACCGCAGGAAGTGTGCGTGGCGGTGTGG | |
| | CGCAAAAACGATGAAAACATTACCCTGGAAACCGTG | |
| | TGCCATGATCCGAAACTGCCGTATCATGATTTTATTC | |
| | TGGAAGATGCGGCGAGCCCGAAATGCATTATGAAAG | |
| | AAAAAAAAAAACCGGGCGAAACCTTTTTTATGTGCAG | |
| | CTGCAGCAGCGATGAATGCAACGATAACATTATTTTT | |
| | AGCGAAGAATATAACACCAGCAACCCGGATATGGAT | |
| | CCGAAAAGCTGCGATAAAACCCATACCTGCCCGCCG | |
| | TGCCCGGCGCCGGAACTGCTGGGCGGCCCGAGCG | |
| | TGTTTCTGTTTCCGCCGAAACCGAAAGATACCCTGAT | |
| | GATTAGCCGCACCCCGGAAGTGACCTGCGTGGTGG | |
| | TGGATGTGAGCCATGAAGATCCGGAAGTGAAATTTA | |
| | ACTGGTATGTGGATGGCGTGGAAGTGCATAACGCGA | |
| | AAACCAAACCGCGCGAAGAACAGTATAACAGCACCT | |
| | ATCGCGTGGTGAGCGTGCTGACCGTGCTGCATCAG | |
| | GATTGGCTGAACGGCAAAGAATATAAATGCAAAGTG | |
| | AGCAACAAAGCGCTGCCGGCGCCGATTGAAAAAACC | |
| | ATTAGCAAAGCGAAAGGCCAGCCGCGCGAACCGCA | |
| | GGTGTATACCCTGCCGCCGAGCCGCGATGAACTGA | |
| | CCAAAAACCAGGTGAGCCTGACCTGCCTGGTGAAAG | |
| | GCTTTTATCCGAGCGATATTGCGGTGGAATGGGAAA | |
| | GCAACGGCCAGCCGGAAAACAACTATAAAACCACCC | |
| | CGCCGGTGCTGGATAGCGATGGCAGCTTTTTTCTGT | |
| | ATAGCAAACTGACCGTGGATAAAAGCCGCTGGCAGC | |
| | AGGGCAACGTGTTTAGCTGCAGCGTGATGCATGAAG | |
| | CGCTGCATAACCATTATACCCAGAAAAGCCTGAGCC | |
| | TGAGCCCGGGCAAAGGAAGCGGAGCCACGAACTT | |
| | CTCTCTGTTAAAGCAAGCAGGAGATGTTGAAGAA | |
| | AACCCCGGGCCTATGGTGAGCAAGGGCGAGGAG | |
| | CTGTTCACCGGGGTGGTGCCCATCCTGGTCGAGC | |
| | TGGACGGCGACGTAAACGGCCACAAGTTCAGCGT | |
| | GTCCGGCGAGGGCGAGGGCGATGCCACCTACGG | |
| | CAAGCTGACCCTGAAGTTCATCTGCACCACCGGC | |
| | AAGCTGCCCGTGCCCTGGCCCACCCTCGTGACCA | |
| | CCCTGACCTACGGCGTGCAGTGCTTCAGCCGCTA | |
| | CCCCGACCACATGAAGCAGCACGACTTCTTCAAG | |
| | TCCGCCATGCCCGAAGGCTACGTCCAGGAGCGCA | |
| | CCATCTTCTTCAAGGACGACGGCAACTACAAGAC | |
| | CCGCGCCGAGGTGAAGTTCGAGGGCGACACCCTG | |
| | GTGAACCGCATCGAGCTGAAGGGCATCGACTTCA | |
| | AGGAGGACGGCAACATCCTGGGGCACAAGCTGG | |
| | AGTACAACTACAACAGCCACAACGTCTATATCAT | |
| | GGCCGACAAGCAGAAGAACGGCATCAAGGTGAA | |
| | CTTCAAGATCCGCCACAACATCGAGGACGGCAGC | |
| | GTGCAGCTCGCCGACCACTACCAGCAGAACACCC | |
| | CCATCGGCGACGGCCCCGTGCTGCTGCCCGACAA | |
| | CCACTACCTGAGCACCCAGTCCGCCCTGAGCAAA | |
| | GACCCCAACGAGAAGCGCGATCACATGGTCCTGC | |
| | TGGAGTTCGTGACCGCCGCCGGGATCACTCTCGG | |
| | CATGGACGAGCTGTACAAGTAAACCCAGCTTTCT | |
| | TGTACAAAGTGGTGATCCTCAGGTGCAGGCTGCC | |
| | TATCAGAAGGTGGTGGCTGGTGTGGCCAATGCCC | |
| | TGGCTCACAAATACCACTGAGATCTTTTTCCCTCT | |
| | GCCAAAAATTATGGGGACATCATGAAGCCCCTTG | |
| | AGCATCTGACTTCTGGCTAATAAAGGAAATTTAT | |
| | TTTCATTGCAATAGTGTGTTGGAATTTTTTGTGTC | |
| | TCTCACTCGGAAGGACATATGGGAGGGCAAATCA | |
| | TTTAAAACATCAGAATGAGTATTTGGTTTAGAGT | |
| | TTGGCAACATATGCCCATATGCTGGCTGCCATGA | |
| | ACAAAGGTTGGCTATAAAGAGGTCATCAGTATAT | |
| | GAAACAGCCCCCTGCTGTCCATTCCTTATTCCATA | |
| | GAAAAGCCTTGACTTGAGGTTAGATTTTTTTTATA | |
| | TTTTGTTTTGTGTTATTTTTTTCTTTAACATCCCTA | |
| | AAATTTTCCTTACATGTTTTACTAGCCAGATTTTT | |
| | CCTCCTCTCCTGACTACTCCCAGTCATAGCTGTCC | |
| | CTCTTCTCTTATGGAGATCCCTCGACCTGCAGCCC | |
| | AAGCTTCGCGTTGACATTGATTATTGACTAGTTAT | |
| | TAATAGTAATCAATTACGGGGTCATTAGTTCATA | |
| | GCCCATATATGGAGTTCCGCGTTACATAACTTAC | |
| | GGTAAATGGCCCGCCTGGCTGACCGCCCAACGAC | |
| | CCCCGCCCATTGACGTCAATAATGACGTATGTTC | |
| | CCATAGTAACGCCAATAGGGACTTTCCATTGACG | |
| | TCAATGGGTGGAGTATTTACGGTAAACTGCCCAC | |
| | TTGGCAGTACATCAAGTGTATCATATGCCAAGTA | |
| | CGCCCCCTATTGACGTCAATGACGGTAAATGGCC | |
| | CGCCTGGCATTATGCCCAGTACATGACCTTATGG | |
| | GACTTTCCTACTTGGCAGTACATCTACGTATTAGT | |
| | CATCGCTATTACCATGGTGATGCGGTTTTGGCAG | |
| | TACATCAATGGGCGTGGATAGCGGTTTGACTCAC | |
| | GGGGATTTCCAAGTCTCCACCCCATTGACGTCAA | |
| | TGGGAGTTTGTTTTGGCACCAAAATCAACGGGAC | |
| | TTTCCAAAATGTCGTAACAACTCCGCCCCATTGA | |
| | CGCAAATGGGCGGTAGGCGTGTACGGTGGGAGG | |
| | TCTATATAAGCAGAGCTCTCTGGCTAACTAGAGA | |
| | ACCCACTGCGCCACCATGGCCAAGCCTTTGTCTC | |
| | AAGAAGAATCCACCCTCATTGAAAGAGCAACGG | |
| | CTACAATCAACAGCATCCCCATCTCTGAAGACTA | |
| | CAGCGTCGCCAGCGCAGCTCTCTCTAGCGACGGC | |
| | CGCATCTTCACTGGTGTCAATGTATATCATTTTAC | |
| | TGGGGGACCTTGTGCAGAACTCGTGGTGCTGGGC | |
| | ACTGCTGCTGCTGCGGCAGCTGGCAACCTGACTT | |
| | GTATCGTCGCGATCGGAAATGAGAACAGGGGCA | |
| | TCTTGAGCCCCTGCGGACGGTGCCGACAGGTGCT | |
| | TCTCGATCTGCATCCTGGGATCAAAGCCATAGTG | |
| | AAGGACAGTGATGGACAGCCGACGGCAGTTGGG | |
| | ATTCGTGAATTGCTGCCCTCTGGTTATGTGTGGGA | |
| | GGGCTAACTCGAGTCTAGAGGGCCCGTTTAAACC | |
| | CGCTGATCAGCCTCGACTGTGCCTTCTAGTTGCCA | |
| | GCCATCTGTTGTTTGCCCCTCCCCCGTGCCTTCCT | |
| | TGACCCTGGAAGGTGCCACTCCCACTGTCCTTTCC | |
| | TAATAAAATGAGGAAATTGCATCGCATTGTCTGA | |
| | GTAGGTGTCATTCTATTCTGGGGGGTGGGGTGGG | |
| | GCAGGACAGCAAGGGGGAGGATTGGGAAGACAA | |
| | TAGCAGGCATGCTGGGGATGCGGTGGGCTCTATG | |
| | GCTCGAGTTAATTAACGAGAGCATAATATTGATA | |
| | TGTGCCAAAGTTGTTTCTGACTGACTAATAAGTA | |
| | TAATTTGTTTCTATTATGTATAGGTTAAGCTAATT | |
| | ACTTATTTTATAATACAACATGACTGTTTTTAAAG | |
| | TACAAAATAAGTTTATTTTTGTAAAAGAGAGAAT | |
| | GTTTAAAAGTTTTGTTACTTTATAGAAGAAATTTT | |
| | GAGTTTTTGTTTTTTTTTAATAAATAAATAAACAT | |
| | AAATAAATTGTTTGTTGAATTTATTATTAGTATGT | |
| | AAGTGTAAATATAATAAAACTTAATATCTATTCA | |
| | AATTAATAAATAAACCTCGATATACAGACCGATA | |
| | AAACACATGCGTCAATTTTACGCATGATTATCTTT | |
| | AACGTACGTCACAATATGATTATCTTTCTAGGGTT | |
| | AAATAATAGTTTCTAATTTTTTTATTATTCAGCCT | |
| | GCTGTCGTGAATACCGAGCTCCAATTCGCCCTAT | |
| | AGTGAGTCGTATTACAATTCACTGGCCGTCGTTTT | |
| | ACAACGTCGTGACTGGGAAAACCCTGGCGTTACC | |
| | CAACTTAATCGCCTTGCAGCACATCCCCCTTTCGC | |
| | CAGCTGGCGTAATAGCGAAGAGGCCCGCACCGA | |
| | TCGCCCTTCCCAACAGTTGCGCAGCCTGAATGGC | |
| | GAATGGGACGCGCCCTGTAGCGGCGCATTAAGCG | |
| | CGGCGGGTGTGGTGGTTACGCGCAGCGTGACCGC | |
| | TACACTTGCCAGCGCCCTAGCGCCCGCTCCTTTCG | |
| | CTTTCTTCCCTTCCTTTCTCGCCACGTTCGCCGGC | |
| | TTTCCCCGTCAAGCTCTAAATCGGGGGCTCCCTTT | |
| | AGGGTTCCGATTTAGTGCTTTACGGCACCTCGAC | |
| | CCCAAAAAACTTGATTAGGGTGATGGTTCACGTA | |
| | GTGGGCCATCGCCCTGATAGACGGTTTTTCGCCC | |
| | TTTGACGTTGGAGTCCACGTTCTTTAATAGTGGAC | |
| | TCTTGTTCCAAACTGGAACAACACTCAACCCTAT | |
| | CTCGGTCTATTCTTTTGATTTATAAGGGATTTTGC | |
| | CGATTTCGGCCTATTGGTTAAAAAATGAGCTGAT | |
| | TTAACAAAAATTTAACGCGAATTTTAACAAAATA | |
| | TTAACGCTTACAATTTAGGTGGCACTTTTCGGGG | |
| | AAATGTGCGCGGAACCCCTATTTGTTTATTTTTCT | |
| | AAATACATTCAAATATGTATCCGCTCATGAGACA | |
| | ATAACCCTGATAAATGCTTCAATAATATTGAAAA | |
| | AGGAAGAGTATGAGTATTCAACATTTCCGTGTCG | |
| | CCCTTATTCCCTTTTTTGCGGCATTTTGCCTTCCTG | |
| | TTTTTGCTCACCCAGAAACGCTGGTGAAAGTAAA | |
| | AGATGCTGAAGATCAGTTGGGTGCACGAGTGGGT | |
| | TACATCGAACTGGATCTCAACAGCGGTAAGATCC | |
| | TTGAGAGTTTTCGCCCCGAAGAACGTTTTCCAAT | |
| | GATGAGCACTTTTAAAGTTCTGCTATGTGGCGCG | |
| | GTATTATCCCGTATTGACGCCGGGCAAGAGCAAC | |
| | TCGGTCGCCGCATACACTATTCTCAGAATGACTT | |
| | GGTTGAGTACTCACCAGTCACAGAAAAGCATCTT | |
| | ACGGATGGCATGACAGTAAGAGAATTATGCAGT | |
| | GCTGCCATAACCATGAGTGATAACACTGCGGCCA | |
| | ACTTACTTCTGACAACGATCGGAGGACCGAAGGA | |
| | GCTAACCGCTTTTTTGCACAACATGGGGGATCAT | |
| | GTAACTCGCCTTGATCGTTGGGAACCGGAGCTGA | |
| | ATGAAGCCATACCAAACGACGAGCGTGACACCA | |
| | CGATGCCTGTAGCAATGGCAACAACGTTGCGCAA | |
| | ACTATTAACTGGCGAACTACTTACTCTAGCTTCCC | |
| | GGCAACAATTAATAGACTGGATGGAGGCGGATA | |
| | AAGTTGCAGGACCACTTCTGCGCTCGGCCCTTCC | |
| | GGCTGGCTGGTTTATTGCTGATAAATCTGGAGCC | |
| | GGTGAGCGTGGGTCTCGCGGTATCATTGCAGCAC | |
| | TGGGGCCAGATGGTAAGCCCTCCCGTATCGTAGT | |
| | TATCTACACGACGGGGAGTCAGGCAACTATGGAT | |
| | GAACGAAATAGACAGATCGCTGAGATAGGTGCC | |
| | TCACTGATTAAGCATTGGTAACTGTCAGACCAAG | |
| | TTTACTCATATATACTTTAGATTGATTTAAAACTT | |
| | CATTTTTAATTTAAAAGGATCTAGGTGAAGATCC | |
| | TTTTTGATAATCTCATGACCAAAATCCCTTAACGT | |
| | GAGTTTTCGTTCCACTGAGCGTCAGACCCCGTAG | |
| | AAAAGATCAAAGGATCTTCTTGAGATCCTTTTTTT | |
| | CTGCGCGTAATCTGCTGCTTGCAAACAAAAAAAC | |
| | CACCGCTACCAGCGGTGGTTTGTTTGCCGGATCA | |
| | AGAGCTACCAACTCTTTTTCCGAAGGTAACTGGC | |
| | TTCAGCAGAGCGCAGATACCAAATACTGTTCTTC | |
| | TAGTGTAGCCGTAGTTAGGCCACCACTTCAAGAA | |
| | CTCTGTAGCACCGCCTACATACCTCGCTCTGCTAA | |
| | TCCTGTTACCAGTGGCTGCTGCCAGTGGCGATAA | |
| | GTCGTGTCTTACCGGGTTGGACTCAAGACGATAG | |
| | TTACCGGATAAGGCGCAGCGGTCGGGCTGAACG | |
| | GGGGGTTCGTGCACACAGCCCAGCTTGGAGCGAA | |
| | CGACCTACACCGAACTGAGATACCTACAGCGTGA | |
| | GCTATGAGAAAGCGCCACGCTTCCCGAAGGGAG | |
| | AAAGGCGGACAGGTATCCGGTAAGCGGCAGGGT | |
| | CGGAACAGGAGAGCGCACGAGGGAGCTTCCAGG | |
| | GGGAAACGCCTGGTATCTTTATAGTCCTGTCGGG | |
| | TTTCGCCACCTCTGACTTGAGCGTCGATTTTTGTG | |
| | ATGCTCGTCAGGGGGGCGGAGCCTATGGAAAAA | |
| | CGCCAGCAACGCGGCCTTTTTACGGTTCCTGGCC | |
| | TTTTGCTGGCCTTTTGCTCACATGTTCTTTCCTGC | |
| | GTTATCCCCTGATTCTGTGGATAACCGTATTACCG | |
| | CCTTTGAGTGAGCTGATACCGCTCGCCGCAGCCG | |
| | AACGACCGAGCGCAGCGAGTCAGTGAGCGAGGA | |
| | AGCGGAAGAGCGCCCAATACGCAAACCGCCTCTC | |
| | CCCGCGCGTTGGCCGATTCATTAATGCAGCTGGC | |
| | ACGACAGGTTTCCCGACTGGAAAGCGGGCAGTG | |
| | AGCGCAACGCAATTAATGTGAGTTAGCTCACTCA | |
| | TTAGGCACCCCAGGCTTTACACTTTATGCTTCCGG | |
| | CTCGTATGTTGTGTGGAATTGTGAGCGGATAACA | |
| | ATTTCACACAGGAAACAGCTATGACCATGATTAC | |
| | GCCAAGCTCGAAATTAACCCTCACTAAAGGGAAC | |
| | AAAAGCTGGTACCTCGCGCGACTTGGTTTGCCAT | |
| | TCTTTAGCGCGCGTCGCGTCACACAGCTTGGCCA | |
| | CAATGTGGTTTTTGTCAAACGAAGATTCTATGAC | |
| | GTGTTTAAAGTTTAGGTCGAGTAAAGCGCAAATC | |
| | TTTT | |
| 7 | CCGGGTCCGCTCAGCCACTGGCACGCATGTGAGA | CCL2 promoter |
| | GCGCCACTCTTTTTATCAAGAGTCTGCTGAAATG | |
| | AATTCTAAGGGCTTTCAGATTTTATCGCTTCGATC | |
| | ACACTGTTTGTGAAGAAATCTAAACCTGGAAAGC | |
| | TGAATTAAGCCAGACATTCCAGTTGGCTCACTCA | |
| | TGGTCCCTGGAAATCACGAAGGAAACAGGGCAG | |
| | AGAGCTACCAGGAGTAGTAAACGCTAACACACA | |
| | GGCTTCTAATTCATCATGGCTTAACCAAAAGGCA | |
| | TGTTTCTCCATGGCCCGTTAGCTGTCTGTTAGTTT | |
| | GAAGGTAATGTGATGATAGCCAGGCTGGAGCTAC | |
| | CTGGGTCCTGTTCCAGGGACTCTTAGGGCAGTTA | |
| | CCTACATGACCCTTCTGGACCTCAGCTGCCTGATC | |
| | TGAGAATGGGGAGTAACAGCATCTACTTACCAGA | |
| | GCTGGGGAAATTAGAGACTGCACTGTGAAGGGTT | |
| | CCAACCACTCCTGGCCCAGAGTAAGCACTAGGTG | |
| | AGTTTTATATAGAAATTTCTTCTGCACCATGAGCT | |
| | CATGTATATGGCTTTCCAGGTCTGGACTTTTACAA | |
| | CTCAGACAGTTCATATCAATGTCACAAAGCTTCT | |
| | TAGTGCTGCAAAATATCTGGTAATCACCAAGTGG | |
| | AGAGAATGCTGTATTCAACAAGGCCTGATAACCA | |
| | AGGACTCAGCCGACTTACTGGGGGTCCTTTCCCA | |
| | GCTGCAAGGTTCCTTGAGCCAGGGGCAAGCTAGG | |
| | CTGTGTTTCCAAGCATCTTCTTCCTAAGGACTTTA | |
| | GGTTCCTTGGCCACTTCCTTTTATTTCAGTGAAAG | |
| | CAGAGCCACTCCATTCACACTTGTGGTCACAGTA | |
| | GTACAATTACTGCCAATTCTTCCCTCTTTCCCCCC | |
| | CCCCCCCTACTCCCTGCGCAGCTTCATTTGCTCCC | |
| | AGGAGTGGCTAGAAAAATACCAAATTCCAACCC | |
| | ACAGTTTCTCTCTTCCACTTCCTGGAAACACCCGA | |
| | GGGCTCTGCACTTACTCAGCGGATTCAACTTCCA | |
| | CTTTCCATCACTTATCCAGGGTGATGCTACTCCTT | |
| | GGCACCAAGCACCCTGCCTGACTCCACCCCCCTG | |
| | GCTTACAATAAAAGGCTGCCTCAGAGCAGCCAGA | |
| | AGTGCAC | |
| 8 | ATGGATCGCCTGCAGACCGCGCTGCTGGTGGTGC | CCL22 |
| | TGGTGCTGCTGGCGGTGGCGCTGCAGGCGACCGA | |
| | AGCGGGCCCGTATGGCGCGAACATGGAAGATAG | |
| | CGTGTGCTGCCGCGATTATGTGCGCTATCGCCTG | |
| | CCGCTGCGCGTGGTGAAACATTTTTATTGGACCA | |
| | GCGATAGCTGCCCGCGCCCGGGCGTGGTGCTGCT | |
| | GACCTTTCGCGATAAAGAAATTTGCGCGGATCCG | |
| | CGCGTGCCGTGGGTGAAAATGATTCTGAACAAAC | |
| | TGAGCCAGT | |
| 9 | TTAATTAAGCTAGCTCAGACCGTTAGACGCCAGG | BRE promoter |
| | ACGGGCTGTCAGGCTGGCGCCGCGGCGCCAGCCT | |
| | GACAGCCCGTCCTGGCGTCTAACGGTCTGAGCTA | |
| | GCCCGGGCTCGAGATCTGGGCTATAAAAGGGGGT | |
| | GGGGGCGCGTTCGTCCTCACTCTCTTCCAAGCTTG | |
| | GCATTCCGGTACTGTTGGTAAAGCCACC | |
| 10 | ATGGGCGCGGCGGCGAAACTGGCGTTTGCGGTGT | ActRIIA-Fc |
| | TTCTGATTAGCTGCAGCAGCGGCGCGATTCTGGG | |
| | CCGCAGCGAAACCCAGGAATGCCTGTTTTTTAAC | |
| | GCGAACTGGGAAAAAGATCGCACCAACCAGACC | |
| | GGCGTGGAACCGTGCTATGGCGATAAAGATAAA | |
| | CGCCGCCATTGCTTTGCGACCTGGAAAAACATTA | |
| | GCGGCAGCATTGAAATTGTGAAACAGGGCTGCTG | |
| | GCTGGATGATATTAACTGCTATGATCGCACCGAT | |
| | TGCGTGGAAAAAAAAGATAGCCCGGAAGTGTAT | |
| | TTTTGCTGCTGCGAAGGCAACATGTGCAACGAAA | |
| | AATTTAGCTATTTTCCGGAAATGGAAGTGACCCA | |
| | GCCGACCAGCAACCCGGTGACCCCGAAACCGCC | |
| | GATTGAAGGCCGCATGGATCCGAAAAGCTGCGAT | |
| | AAAACCCATACCTGCCCGCCGTGCCCGGCGCCGG | |
| | AACTGCTGGGCGGCCCGAGCGTGTTTCTGTTTCC | |
| | GCCGAAACCGAAAGATACCCTGATGATTAGCCGC | |
| | ACCCCGGAAGTGACCTGCGTGGTGGTGGATGTGA | |
| | GCCATGAAGATCCGGAAGTGAAATTTAACTGGTA | |
| | TGTGGATGGCGTGGAAGTGCATAACGCGAAAAC | |
| | CAAACCGCGCGAAGAACAGTATAACAGCACCTA | |
| | TCGCGTGGTGAGCGTGCTGACCGTGCTGCATCAG | |
| | GATTGGCTGAACGGCAAAGAATATAAATGCAAA | |
| | GTGAGCAACAAAGCGCTGCCGGCGCCGATTGAA | |
| | AAAACCATTAGCAAAGCGAAAGGCCAGCCGCGC | |
| | GAACCGCAGGTGTATACCCTGCCGCCGAGCCGCG | |
| | ATGAACTGACCAAAAACCAGGTGAGCCTGACCTG | |
| | CCTGGTGAAAGGCTTTTATCCGAGCGATATTGCG | |
| | GTGGAATGGGAAAGCAACGGCCAGCCGGAAAAC | |
| | AACTATAAAACCACCCCGCCGGTGCTGGATAGCG | |
| | ATGGCAGCTTTTTTCTGTATAGCAAACTGACCGT | |
| | GGATAAAAGCCGCTGGCAGCAGGGCAACGTGTTT | |
| | AGCTGCAGCGTGATGCATGAAGCGCTGCATAACC | |
| | ATTATACCCAGAAAAGCCTGAGCCTGAGCCCGGG | |
| | CAAAG | |
| 11 | CCCGGGCTCGAGAGCCAGACAAAAAGCCAGACA | CAGA promoter |
| | TTTAGCCAGACACTCGAGAGCCAGACAAAAAGC | |
| | CAGACATTTAGCCAGACACTCGAGAGCCAGACA | |
| | AAAAGCCAGACATTTAGCCAGACACTCGAGTGTC | |
| | TGGCTAAATGTCTGGCTTTTTGTCTGGCTCTCGAG | |
| | ATCTGGGCTATAAAAGGGGGTGGGGGCGCGTTCG | |
| | TCCTCACTCTCTTCCAAGCTTAGGCGTGTACGGTG | |
| | GGAGGCCTATATAAGCAGAGCTCGTTTAGTGAAC | |
| | CGTCAGATCGCACTAGTC | |
| 12 | ATGGGCCGCGGCCTGCTGCGCGGCCTGTGGCCGC | TGFBRII-Fc |
| | TGCATATTGTGCTGTGGACCCGCATTGCGAGCAC | |
| | CATTCCGCCGCATGTGCAGAAAAGCGTGAACAAC | |
| | GATATGATTGTGACCGATAACAACGGCGCGGTGA | |
| | AATTTCCGCAGCTGTGCAAATTTTGCGATGTGCG | |
| | CTTTAGCACCTGCGATAACCAGAAAAGCTGCATG | |
| | AGCAACTGCAGCATTACCAGCATTTGCGAAAAAC | |
| | CGCAGGAAGTGTGCGTGGCGGTGTGGCGCAAAA | |
| | ACGATGAAAACATTACCCTGGAAACCGTGTGCCA | |
| | TGATCCGAAACTGCCGTATCATGATTTTATTCTGG | |
| | AAGATGCGGCGAGCCCGAAATGCATTATGAAAG | |
| | AAAAAAAAAAACCGGGCGAAACCTTTTTTATGTG | |
| | CAGCTGCAGCAGCGATGAATGCAACGATAACATT | |
| | ATTTTTAGCGAAGAATATAACACCAGCAACCCGG | |
| | ATATGGATCCGAAAAGCTGCGATAAAACCCATAC | |
| | CTGCCCGCCGTGCCCGGCGCCGGAACTGCTGGGC | |
| | GGCCCGAGCGTGTTTCTGTTTCCGCCGAAACCGA | |
| | AAGATACCCTGATGATTAGCCGCACCCCGGAAGT | |
| | GACCTGCGTGGTGGTGGATGTGAGCCATGAAGAT | |
| | CCGGAAGTGAAATTTAACTGGTATGTGGATGGCG | |
| | TGGAAGTGCATAACGCGAAAACCAAACCGCGCG | |
| | AAGAACAGTATAACAGCACCTATCGCGTGGTGAG | |
| | CGTGCTGACCGTGCTGCATCAGGATTGGCTGAAC | |
| | GGCAAAGAATATAAATGCAAAGTGAGCAACAAA | |
| | GCGCTGCCGGCGCCGATTGAAAAAACCATTAGCA | |
| | AAGCGAAAGGCCAGCCGCGCGAACCGCAGGTGT | |
| | ATACCCTGCCGCCGAGCCGCGATGAACTGACCAA | |
| | AAACCAGGTGAGCCTGACCTGCCTGGTGAAAGGC | |
| | TTTTATCCGAGCGATATTGCGGTGGAATGGGAAA | |
| | GCAACGGCCAGCCGGAAAACAACTATAAAACCA | |
| | CCCCGCCGGTGCTGGATAGCGATGGCAGCTTTTT | |
| | TCTGTATAGCAAACTGACCGTGGATAAAAGCCGC | |
| | TGGCAGCAGGGCAACGTGTTTAGCTGCAGCGTGA | |
| | TGCATGAAGCGCTGCATAACCATTATACCCAGAA | |
| | AAGCCTGAGCCTGAGCCCGGGCAAA | |
| 13 | TTAATTAAGCTAGCTCAGACCGTTAGACGCCAGG | BRE-BMPRIA |
| | ACGGGCTGTCAGGCTGGCGCCGCGGCGCCAGCCT | construct |
| | GACAGCCCGTCCTGGCGTCTAACGGTCTGAGCTA | |
| | GCCCGGGCTCGAGATCTGGGCTATAAAAGGGGGT | |
| | GGGGGCGCGTTCGTCCTCACTCTCTTCCAAGCTTG | |
| | GCATTCCGGTACTGTTGGTAAAGCCACCATGCCG | |
| | CAGCTGTATATTTATATTCGCCTGCTGGGCGCGTA | |
| | TCTGTTTATTATTAGCCGCGTGGGCGGCCAGAAC | |
| | CTGGATAGCATGCTGCATGGCACCGGCATGAAAA | |
| | GCGATAGCGATCAGAAAAAAAGCGAAAACGGCG | |
| | TGACCCTGGCGCCGGAAGATACCCTGCCGTTTCT | |
| | GAAATGCTATTGCAGCGGCCATTGCCCGGATGAT | |
| | GCGATTAACAACACCTGCATTACCAACGGCCATT | |
| | GCTTTGCGATTATTGAAGAAGATGATCAGGGCGA | |
| | AACCACCCTGGCGAGCGGCTGCATGAAATATGAA | |
| | GGCAGCGATTTTCAGTGCAAAGATAGCCCGAAAG | |
| | CGCAGCTGCGCCGCACCATTGAATGCTGCCGCAC | |
| | CAACCTGTGCAACCAGTATCTGCAGCCGACCCTG | |
| | CCGCCGGTGGTGATTGGCCCGTTTTTTGATGGCA | |
| | GCATTCGCATTGAAGGCCGCATGGATCCGAAAAG | |
| | CTGCGATAAAACCCATACCTGCCCGCCGTGCCCG | |
| | GCGCCGGAACTGCTGGGCGGCCCGAGCGTGTTTC | |
| | TGTTTCCGCCGAAACCGAAAGATACCCTGATGAT | |
| | TAGCCGCACCCCGGAAGTGACCTGCGTGGTGGTG | |
| | GATGTGAGCCATGAAGATCCGGAAGTGAAATTTA | |
| | ACTGGTATGTGGATGGCGTGGAAGTGCATAACGC | |
| | GAAAACCAAACCGCGCGAAGAACAGTATAACAG | |
| | CACCTATCGCGTGGTGAGCGTGCTGACCGTGCTG | |
| | CATCAGGATTGGCTGAACGGCAAAGAATATAAAT | |
| | GCAAAGTGAGCAACAAAGCGCTGCCGGCGCCGA | |
| | TTGAAAAAACCATTAGCAAAGCGAAAGGCCAGC | |
| | CGCGCGAACCGCAGGTGTATACCCTGCCGCCGAG | |
| | CCGCGATGAACTGACCAAAAACCAGGTGAGCCT | |
| | GACCTGCCTGGTGAAAGGCTTTTATCCGAGCGAT | |
| | ATTGCGGTGGAATGGGAAAGCAACGGCCAGCCG | |
| | GAAAACAACTATAAAACCACCCCGCCGGTGCTGG | |
| | ATAGCGATGGCAGCTTTTTTCTGTATAGCAAACT | |
| | GACCGTGGATAAAAGCCGCTGGCAGCAGGGCAA | |
| | CGTGTTTAGCTGCAGCGTGATGCATGAAGCGCTG | |
| | CATAACCATTATACCCAGAAAAGCCTGAGCCTGA | |
| | GCCCGGGCAAAG | |
| 14 | CAGTGGCAGCCAGGTTTAGCCCCGGAATTGACTG | BRE-BMPRIA |
| | GATTCCTTTTTTAGGGCCCATTGGTATGGCTTTTT | transposon vector |
| | CCCCGTATCCCCCCAGGTGTCTGCAGGCTCAAAG | (BRE-BMPRIA |
| | AGCAGCGAGAAGCGTTCAGAGGAAAGCGATCCC | construct underlined, |
| | GTGCCACCTTCCCCGTGCCCGGGCTGTCCCCGCA | BRE promoter bold, |
| | CGCTGCCGGCTCGGGGATGCGGGGGGAGCGCCG | BMPRIA-Fc gene |
| | GACCGGAGCGGAGCCCCGGGCGGCTCGCTGCTGC | italicized) |
| | CCCCTAGCGGGGGAGGGACGTAATTACATCCCTG | |
| | GGGGCTTTGGGGGGGGGCTGTCCCTGATATCTAT | |
| | AACAAGAAAATATATATATAATAAGTTATCACGT | |
| | AAGTAGAACATGAAATAACAATATAATTATCGTA | |
| | TGAGTTAAATCTTAAAAGTCACGTAAAAGATAAT | |
| | CATGCGTCATTTTGACTCACGCGGTCGTTATAGTT | |
| | CAAAATCAGTGACACTTACCGCATTGACAAGCAC | |
| | GCCTCACGGGAGCTCCAAGCGGCGACTGAGATGT | |
| | CCTAAATGCACAGCGACGGATTCGCGCTATTTAG | |
| | AAAGAGAGAGCAATATTTCAAGAATGCATGCGTC | |
| | AATTTTACGCAGACTATCTTTCTAGGGTTAATCTA | |
| | GCTGCATCAGGATCATATCGTCGGGTCTTTTTTCC | |
| | GGCTCAGTCATCGCCCAAGCTGGCGCTATCTGGG | |
| | CATCGGGGAGGAAGAAGCCCGTGCCTTTTCCCGC | |
| | GAGGTTGAAGCGGCATGGAAAGAGTTTGCCGAG | |
| | GATGACTGCTGCTGCATTGACGTTGAGCGAAAAC | |
| | GCACGTTTACCATGATGATTCGGGAAGGTGTGGC | |
| | CATGCACGCCTTTAACGGTGAACTGTTCGTTCAG | |
| | GCCACCTGGGATACCAGTTCGTCGCGGCTTTTCC | |
| | GGACACAGTTCCGGATGGTCAGCCCGAAGCGCAT | |
| | CAGCAACCCGAACAATACCGGCGACAGCCGGAA | |
| | CTGCCGTGCCGGTGTGCAGATTAATGACAGCGGT | |
| | GCGGCGCTGGGATATTACGTCAGCGAGGACGGGT | |
| | ATCCTGGCTGGATGCCGCAGAAATGGACATGGAT | |
| | ACCCCGTGAGTTACCCGGCGGGCGCGCTTGGCGT | |
| | AATCATGGTCATAGCTGTTTCCTGTGTGAAATTGT | |
| | TATCCGCTCACAATTCCACACAACATACGAGCCG | |
| | GAAGCATAAAGTGTAAAGCCTGGGGTGCCTAATG | |
| | AGTGAGCTAACTCACATTAATTGCGTTGCGCTCA | |
| | CTGCCCGCTTTCCAGTCGGGAAACCTGTCGTGCC | |
| | AGCTGCATTAATGAATCGGCCAACGCGCGGGGA | |
| | GAGGCGGTTTGCGTATTGGGCGCTCTTCCGCTTCC | |
| | TCGCTCACTGACTCGCTGCGCTCGGTCGTTCGGCT | |
| | GCGGCGAGCGGTATCAGCTCACTCAAAGGCGGTA | |
| | ATACGGTTATCCACAGAATCAGGGGATAACGCAG | |
| | GAAAGAACATGTGAGCAAAAGGCCAGCAAAAGG | |
| | CCAGGAACCGTAAAAAGGCCGCGTTGCTGGCGTT | |
| | TTTCCATAGGCTCCGCCCCCCTGACGAGCATCAC | |
| | AAAAATCGACGCTCAAGTCAGAGGTGGCGAAAC | |
| | CCGACAGGACTATAAAGATACCAGGCGTTTCCCC | |
| | CTGGAAGCTCCCTCGTGCGCTCTCCTGTTCCGACC | |
| | CTGCCGCTTACCGGATACCTGTCCGCCTTTCTCCC | |
| | TTCGGGAAGCGTGGCGCTTTCTCATAGCTCACGC | |
| | TGTAGGTATCTCAGTTCGGTGTAGGTCGTTCGCTC | |
| | CAAGCTGGGCTGTGTGCACGAACCCCCCGTTCAG | |
| | CCCGACCGCTGCGCCTTATCCGGTAACTATCGTCT | |
| | TGAGTCCAACCCGGTAAGACACGACTTATCGCCA | |
| | CTGGCAGCAGCCACTGGTAACAGGATTAGCAGA | |
| | GCGAGGTATGTAGGCGGTGCTACAGAGTTCTTGA | |
| | AGTGGTGGCCTAACTACGGCTACACTAGAAGGAC | |
| | AGTATTTGGTATCTGCGCTCTGCTGAAGCCAGTT | |
| | ACCTTCGGAAAAAGAGTTGGTAGCTCTTGATCCG | |
| | GCAAACAAACCACCGCTGGTAGCGGTGGTTTTTT | |
| | TGTTTGCAAGCAGCAGATTACGCGCAGAAAAAA | |
| | AGGATCTCAAGAAGATCCTTTGATCTTTTCTACG | |
| | GGGTCTGACGCTCAGTGGAACGAAAACTCACGTT | |
| | AAGGGATTTTGGTCATGAGATTATCAAAAAGGAT | |
| | CTTCACCTAGATCCTTTTAAATTAAAAATGAAGTT | |
| | TTAAATCAATCTAAAGTATATATGAGTAAACTTG | |
| | GTCTGACAGTTACCAATGCTTAATCAGTGAGGCA | |
| | CCTATCTCAGCGATCTGTCTATTTCGTTCATCCAT | |
| | AGTTGCCTGACTCCCCGTCGTGTAGATAACTACG | |
| | ATACGGGAGGGCTTACCATCTGGCCCCAGTGCTG | |
| | CAATGATACCGCGAGACCCACGCTCACCGGCTCC | |
| | AGATTTATCAGCAATAAACCAGCCAGCCGGAAG | |
| | GGCCGAGCGCAGAAGTGGTCCTGCAACTTTATCC | |
| | GCCTCCATCCAGTCTATTAATTGTTGCCGGGAAG | |
| | CTAGAGTAAGTAGTTCGCCAGTTAATAGTTTGCG | |
| | CAACGTTGTTGCCATTGCTACAGGCATCGTGGTG | |
| | TCACGCTCGTCGTTTGGTATGGCTTCATTCAGCTC | |
| | CGGTTCCCAACGATCAAGGCGAGTTACATGATCC | |
| | CCCATGTTGTGCAAAAAAGCGGTTAGCTCCTTCG | |
| | GTCCTCCGATCGTTGTCAGAAGTAAGTTGGCCGC | |
| | AGTGTTATCACTCATGGTTATGGCAGCACTGCAT | |
| | AATTCTCTTACTGTCATGCCATCCGTAAGATGCTT | |
| | TTCTGTGACTGGTGAGTACTCAACCAAGTCATTCT | |
| | GAGAATAGTGTATGCGGCGACCGAGTTGCTCTTG | |
| | CCCGGCGTCAATACGGGATAATACCGCGCCACAT | |
| | AGCAGAACTTTAAAAGTGCTCATCATTGGAAAAC | |
| | GTTCTTCGGGGCGAAAACTCTCAAGGATCTTACC | |
| | GCTGTTGAGATCCAGTTCGATGTAACCCACTCGT | |
| | GCACCCAACTGATCTTCAGCATCTTTTACTTTCAC | |
| | CAGCGTTTCTGGGTGAGCAAAAACAGGAAGGCA | |
| | AAATGCCGCAAAAAAGGGAATAAGGGCGACACG | |
| | GAAATGTTGAATACTCATACTCTTCCTTTTTCAAT | |
| | ATTATTGAAGCATTTATCAGGGTTATTGTCTCATG | |
| | AGCGGATACATATTTGAATGTATTTAGAAAAATA | |
| | AACAAATAGGGGTTCCGCGCACATTTCCCCGAAA | |
| | AGTGCCACCTAAATTGTAAGCGTTAATATTTTGTT | |
| | AAAATTCGCGTTAAATTTTTGTTAAATCAGCTCAT | |
| | TTTTTAACCAATAGGCCGAAATCGGCAAAATCCC | |
| | TTATAAATCAAAAGAATAGACCGAGATAGGGTTG | |
| | AGTGTTGTTCCAGTTTGGAACAAGAGTCCACTAT | |
| | TAAAGAACGTGGACTCCAACGTCAAAGGGCGAA | |
| | AAACCGTCTATCAGGGCGATGGCCCACTACGTGA | |
| | ACCATCACCCTAATCAAGTTTTTTGGGGTCGAGG | |
| | TGCCGTAAAGCACTAAATCGGAACCCTAAAGGG | |
| | AGCCCCCGATTTAGAGCTTGACGGGGAAAGCCGG | |
| | CGAACGTGGCGAGAAAGGAAGGGAAGAAAGCGA | |
| | AAGGAGCGGGCGCTAGGGCGCTGGCAAGTGTAG | |
| | CGGTCACGCTGCGCGTAACCACCACACCCGCCGC | |
| | GCTTAATGCGCCGCTACAGGGCGCGTCCCATTCG | |
| | CCATTCAGGCTGCGCAACTGTTGGGAAGGGCGAT | |
| | CGGTGCGGGCCTCTTCGCTATTACGCCAGCTGGC | |
| | GAAAGGGGGATGTGCTGCAAGGCGATTAAGTTG | |
| | GGTAACGCCAGGGTTTTCCCAGTCACGACGTTGT | |
| | AAAACGACGGCCAGTGAGCGCGCCTCGTTCATTC | |
| | ACGTTTTTGAACCCGTGGAGGACGGGCAGACTCG | |
| | CGGTGCAAATGTGTTTTACAGCGTGATGGAGCAG | |
| | ATGAAGATGCTCGACACGCTGCAGAACACGCAG | |
| | CTAGATTAACCCTAGAAAGATAATCATATTGTGA | |
| | CGTACGTTAAAGATAATCATGTGTAAAATTGACG | |
| | CATGTGTTTTATCGGTCTGTATATCGAGGTTTATT | |
| | TATTAATTTGAATAGATATTAAGTTTTATTATATT | |
| | TACACTTACATACTAATAATAAATTCAACAAACA | |
| | ATTTATTTATGTTTATTTATTTATTAAAAAAAACA | |
| | AAAACTCAAAATTTCTTCTATAAAGTAACAAAAC | |
| | TTTTATGAGGGACAGCCCCCCCCCAAAGCCCCCA | |
| | GGGATGTAATTACGTCCCTCCCCCGCTAGGGGGC | |
| | AGCAGCGAGCCGCCCGGGGCTCCGCTCCGGTCCG | |
| | GCGCTCCCCCCGCATCCCCGAGCCGGCAGCGTGC | |
| | GGGGACAGCCCGGGCACGGGGAAGGTGGCACGG | |
| | GATCGCTTTCCTCTGAACGCTTCTCGCTGCTCTTT | |
| | GAGCCTGCAGACACCTGGGGGGATACGGGGAAA | |
| | AGGCCTCCACGGCCTTGCGTTTAAACAGATCTAC | |
| | GCGTTTAATTAAGCTAGCTCAGACCGTTAGAC | |
| | GCCAGGACGGGCTGTCAGGCTGGCGCCGCGG | |
| | CGCCAGCCTGACAGCCCGTCCTGGCGTCTAAC | |
| | GGTCTGAGCTAGCCCGGGCTCGAGATCTGGGC | |
| | TATAAAAGGGGGTGGGGGCGCGTTCGTCCTCA | |
| | CTCTCTTCCAAGCTTGGCATTCCGGTACTGTT | |
| | GGTAAAGCCACCATGCCGCAGCTGTATATTTATAT | |
| | TCGCCTGCTGGGCGCGTATCTGTTTATTATTAGCCG | |
| | CGTGGGCGGCCAGAACCTGGATAGCATGCTGCATG | |
| | GCACCGGCATGAAAAGCGATAGCGATCAGAAAAAAA | |
| | GCGAAAACGGCGTGACCCTGGCGCCGGAAGATACC | |
| | CTGCCGTTTCTGAAATGCTATTGCAGCGGCCATTGC | |
| | CCGGATGATGCGATTAACAACACCTGCATTACCAAC | |
| | GGCCATTGCTTTGCGATTATTGAAGAAGATGATCAGG | |
| | GCGAAACCACCCTGGCGAGCGGCTGCATGAAATATG | |
| | AAGGCAGCGATTTTCAGTGCAAAGATAGCCCGAAAG | |
| | CGCAGCTGCGCCGCACCATTGAATGCTGCCGCACC | |
| | AACCTGTGCAACCAGTATCTGCAGCCGACCCTGCCG | |
| | CCGGTGGTGATTGGCCCGTTTTTTGATGGCAGCATT | |
| | CGCATTGAAGGCCGCATGGATCCGAAAAGCTGCGAT | |
| | AAAACCCATACCTGCCCGCCGTGCCCGGCGCCGGA | |
| | ACTGCTGGGCGGCCCGAGCGTGTTTCTGTTTCCGCC | |
| | GAAACCGAAAGATACCCTGATGATTAGCCGCACCCC | |
| | GGAAGTGACCTGCGTGGTGGTGGATGTGAGCCATG | |
| | AAGATCCGGAAGTGAAATTTAACTGGTATGTGGATG | |
| | GCGTGGAAGTGCATAACGCGAAAACCAAACCGCGC | |
| | GAAGAACAGTATAACAGCACCTATCGCGTGGTGAGC | |
| | GTGCTGACCGTGCTGCATCAGGATTGGCTGAACGGC | |
| | AAAGAATATAAATGCAAAGTGAGCAACAAAGCGCTG | |
| | CCGGCGCCGATTGAAAAAACCATTAGCAAAGCGAAA | |
| | GGCCAGCCGCGCGAACCGCAGGTGTATACCCTGCC | |
| | GCCGAGCCGCGATGAACTGACCAAAAACCAGGTGA | |
| | GCCTGACCTGCCTGGTGAAAGGCTTTTATCCGAGCG | |
| | ATATTGCGGTGGAATGGGAAAGCAACGGCCAGCCG | |
| | GAAAACAACTATAAAACCACCCCGCCGGTGCTGGAT | |
| | AGCGATGGCAGCTTTTTTCTGTATAGCAAACTGACCG | |
| | TGGATAAAAGCCGCTGGCAGCAGGGCAACGTGTTTA | |
| | GCTGCAGCGTGATGCATGAAGCGCTGCATAACCATT | |
| | ATACCCAGAAAAGCCTGAGCCTGAGCCCGGGCAAA | |
| | GCTACTAACTTCAGCCTGCTGAAGCAGGCTGGCG | |
| | ACGTGGAGGAGAACCCTGGACCTATGGAAGATG | |
| | CCAAAAACATTAAGAAGGGCCCAGCGCCATTCTA | |
| | CCCACTCGAAGACGGGACCGCCGGCGAGCAGCT | |
| | GCACAAAGCCATGAAGCGCTACGCCCTGGTGCCC | |
| | GGCACCATCGCCTTTACCGACGCACATATCGAGG | |
| | TGGACATTACCTACGCCGAGTACTTCGAGATGAG | |
| | CGTTCGGCTGGCAGAAGCTATGAAGCGCTATGGG | |
| | CTGAATACAAACCATCGGATCGTGGTGTGCAGCG | |
| | AGAATAGCTTGCAGTTCTTCATGCCCGTGTTGGG | |
| | TGCCCTGTTCATCGGTGTGGCTGTGGCCCCAGCT | |
| | AACGACATCTACAACGAGCGCGAGCTGCTGAAC | |
| | AGCATGGGCATCAGCCAGCCCACCGTCGTATTCG | |
| | TGAGCAAGAAAGGGCTGCAAAAGATCCTCAACG | |
| | TGCAAAAGAAGCTACCGATCATACAAAAGATCAT | |
| | CATCATGGATAGCAAGACCGACTACCAGGGCTTC | |
| | CAAAGCATGTACACCTTCGTGACTTCCCATTTGCC | |
| | ACCCGGCTTCAACGAGTACGACTTCGTGCCCGAG | |
| | AGCTTCGACCGGGACAAAACCATCGCCCTGATCA | |
| | TGAACAGTAGTGGCAGTACCGGATTGCCCAAGGG | |
| | CGTAGCCCTACCGCACCGCACCGCTTGTGTCCGA | |
| | TTCAGTCATGCCCGCGACCCCATCTTCGGCAACC | |
| | AGATCATCCCCGACACCGCTATCCTCAGCGTGGT | |
| | GCCATTTCACCACGGCTTCGGCATGTTCACCACG | |
| | CTGGGCTACTTGATCTGCGGCTTTCGGGTCGTGCT | |
| | CATGTACCGCTTCGAGGAGGAGCTATTCTTGCGC | |
| | AGCTTGCAAGACTATAAGATTCAATCTGCCCTGC | |
| | TGGTGCCCACACTATTTAGCTTCTTCGCTAAGAGC | |
| | ACTCTCATCGACAAGTACGACCTAAGCAACTTGC | |
| | ACGAGATCGCCAGCGGCGGGGCGCCGCTCAGCA | |
| | AGGAGGTAGGTGAGGCCGTGGCCAAACGCTTCC | |
| | ACCTACCAGGCATCCGCCAGGGCTACGGCCTGAC | |
| | AGAAACAACCAGCGCCATTCTGATCACCCCCGAA | |
| | GGGGACGACAAGCCTGGCGCAGTAGGCAAGGTG | |
| | GTGCCCTTCTTCGAGGCTAAGGTGGTGGACTTGG | |
| | ACACCGGTAAGACACTGGGTGTGAACCAGCGCG | |
| | GCGAGCTGTGCGTCCGTGGCCCCATGATCATGAG | |
| | CGGCTACGTTAACAACCCCGAGGCTACAAACGCT | |
| | CTCATCGACAAGGACGGCTGGCTGCACAGCGGCG | |
| | ACATCGCCTACTGGGACGAGGACGAGCACTTCTT | |
| | CATCGTGGACCGGCTGAAGAGCCTGATCAAATAC | |
| | AAGGGCTACCAGGTAGCCCCAGCCGAACTGGAG | |
| | AGCATCCTGCTGCAACACCCCAACATCTTCGACG | |
| | CCGGGGTCGCCGGCCTGCCCGACGACGATGCCGG | |
| | CGAGCTGCCCGCCGCAGTCGTCGTGCTGGAACAC | |
| | GGTAAAACCATGACCGAGAAGGAGATCGTGGAC | |
| | TATGTGGCCAGCCAGGTTACAACCGCCAAGAAGC | |
| | TGCGCGGTGGTGTTGTGTTCGTGGACGAGGTGCC | |
| | TAAAGGACTGACCGGCAAGTTGGACGCCCGCAA | |
| | GATCCGCGAGATTCTCATTAAGGCCAAGAAGGGC | |
| | GGCAAGATCGCCGTGTAAATTTAAATCGGCCTCG | |
| | ACTGTGCCTTCTAGTTGCCAGCCATCTGTTGTTTG | |
| | CCCCTCCCCCGTGCCTTCCTTGACCCTGGAAGGTG | |
| | CCACTCCCACTGTCCTTTCCTAATAAAATGAGGA | |
| | AATTGCATCGCATTGTCTGAGTAGGTGTCATTCTA | |
| | TTCTGGGGGGTGGGGTGGGGCAGGACAGCAAGG | |
| | GGGAGGATTGGGAAGACAATAGCAGGCATGCTG | |
| | GGGACGCGGCCGCAATTTAACGCGAATTAATTCT | |
| | GTGGAATGTGTGTCAGTTAGGGTGTGGAAAGTCC | |
| | CCAGGCTCCCCAGCAGGCAGAAGTATGCAAAGC | |
| | ATGCATCTCAATTAGTCAGCAACCAGGTGTGGAA | |
| | AGTCCCCAGGCTCCCCAGCAGGCAGAAGTATGCA | |
| | AAGCATGCATCTCAATTAGTCAGCAACCATAGTC | |
| | CCGCCCCTAACTCCGCCCATCCCGCCCCTAACTCC | |
| | GCCCAGTTCCGCCCATTCTCCGCCCCATGGCTGA | |
| | CTAATTTTTTTTATTTATGCAGAGGCCGAGGCCGC | |
| | CTCTGCCTCTGAGCTATTCCAGAAGTAGTGAGGA | |
| | GGCTTTTTTGGAGGCCTAGGCTTTTGCAAAAAGC | |
| | TCCCGGGAGCTTGTATATCCATTTTCGGATCTGAT | |
| | CAGCACGTGATGAAAAAGCCTGAACTCACCGCG | |
| | ACGTCTGTCGAGAAGTTTCTGATCGAAAAGTTCG | |
| | ACAGCGTCTCCGACCTGATGCAGCTCTCGGAGGG | |
| | CGAAGAATCTCGTGCTTTCAGCTTCGATGTAGGA | |
| | GGGCGTGGATATGTCCTGCGGGTAAATAGCTGCG | |
| | CCGATGGTTTCTACAAAGATCGTTATGTTTATCGG | |
| | CACTTTGCATCGGCCGCGCTCCCGATTCCGGAAG | |
| | TGCTTGACATTGGGGAATTCAGCGAGAGCCTGAC | |
| | CTATTGCATCTCCCGCCGTGCACAGGGTGTCACG | |
| | TTGCAAGACCTGCCTGAAACCGAACTGCCCGCTG | |
| | TTCTGCAGCCGGTCGCGGAGGCCATGGATGCGAT | |
| | CGCTGCGGCCGATCTTAGCCAGACGAGCGGGTTC | |
| | GGCCCATTCGGACCGCAAGGAATCGGTCAATACA | |
| | CTACATGGCGTGATTTCATATGCGCGATTGCTGA | |
| | TCCCCATGTGTATCACTGGCAAACTGTGATGGAC | |
| | GACACCGTCAGTGCGTCCGTCGCGCAGGCTCTCG | |
| | ATGAGCTGATGCTTTGGGCCGAGGACTGCCCCGA | |
| | AGTCCGGCACCTCGTGCACGCGGATTTCGGCTCC | |
| | AACAATGTCCTGACGGACAATGGCCGCATAACAG | |
| | CGGTCATTGACTGGAGCGAGGCGATGTTCGGGGA | |
| | TTCCCAATACGAGGTCGCCAACATCTTCTTCTGG | |
| | AGGCCGTGGTTGGCTTGTATGGAGCAGCAGACGC | |
| | GCTACTTCGAGCGGAGGCATCCGGAGCTTGCAGG | |
| | ATCGCCGCGGCTCCGGGCGTATATGCTCCGCATT | |
| | GGTCTTGACCAACTCTATCAGAGCTTGGTTGACG | |
| | GCAATTTCGATGATGCAGCTTGGGCGCAGGGTCG | |
| | ATGCGACGCAATCGTCCGATCCGGAGCCGGGACT | |
| | GTCGGGCGTACACAAATCGCCCGCAGAAGCGCG | |
| | GCCGTCTGGACCGATGGCTGTGTAGAAGTACTCG | |
| | CCGATAGTGGAAACCGACGCCCCAGCACTCGTCC | |
| | GAGGGCAAAGGAATAGGTCGACAATCAACCTCT | |
| | GGATTACAAAATTTGTGAAAGATTGACTGGTATT | |
| | CTTAACTATGTTGCTCCTTTTACGCTATGTGGATA | |
| | CGCTGCTTTAATGCCTTTGTATCATGCGTTAACTA | |
| | AACTTGTTTATTGCAGCTTATAATGGTTACAAATA | |
| | AAGCAATAGCATCACAAATTTCACAAATAAAGCA | |
| | TTTTTTTCACTGCATTCTAGTTGTGGTTTGTCCAA | |
| | ACTCATCAATGTATCTTATCATGTCTGGAATTGAC | |
| | TCAAATGATGTCAATTAGTCTATCAGAAGCTCAT | |
| | CTGGTCTCCCTTCCGGGGGACAAGACATCCCTGT | |
| | TTAATATTTAAACAGCAGTGTTCCCAAACTGGGT | |
| | TCTTATATCCCTTGCTCTGGTCAACCAGGTTGCAG | |
| | GGTTTCCTGTCCTCACAGGAACGAAGTCCCTAAA | |
| | GAAA | |
| 15 | ATGCCGCAGCTGTATATTTATATTCGCCTGCTGGG | BMPRIA-Fc |
| | CGCGTATCTGTTTATTATTAGCCGCGTGGGCGGC | |
| | CAGAACCTGGATAGCATGCTGCATGGCACCGGCA | |
| | TGAAAAGCGATAGCGATCAGAAAAAAAGCGAAA | |
| | ACGGCGTGACCCTGGCGCCGGAAGATACCCTGCC | |
| | GTTTCTGAAATGCTATTGCAGCGGCCATTGCCCG | |
| | GATGATGCGATTAACAACACCTGCATTACCAACG | |
| | GCCATTGCTTTGCGATTATTGAAGAAGATGATCA | |
| | GGGCGAAACCACCCTGGCGAGCGGCTGCATGAA | |
| | ATATGAAGGCAGCGATTTTCAGTGCAAAGATAGC | |
| | CCGAAAGCGCAGCTGCGCCGCACCATTGAATGCT | |
| | GCCGCACCAACCTGTGCAACCAGTATCTGCAGCC | |
| | GACCCTGCCGCCGGTGGTGATTGGCCCGTTTTTTG | |
| | ATGGCAGCATTCGCATTGAAGGCCGCATGGATCC | |
| | GAAAAGCTGCGATAAAACCCATACCTGCCCGCCG | |
| | TGCCCGGCGCCGGAACTGCTGGGCGGCCCGAGCG | |
| | TGTTTCTGTTTCCGCCGAAACCGAAAGATACCCT | |
| | GATGATTAGCCGCACCCCGGAAGTGACCTGCGTG | |
| | GTGGTGGATGTGAGCCATGAAGATCCGGAAGTG | |
| | AAATTTAACTGGTATGTGGATGGCGTGGAAGTGC | |
| | ATAACGCGAAAACCAAACCGCGCGAAGAACAGT | |
| | ATAACAGCACCTATCGCGTGGTGAGCGTGCTGAC | |
| | CGTGCTGCATCAGGATTGGCTGAACGGCAAAGAA | |
| | TATAAATGCAAAGTGAGCAACAAAGCGCTGCCG | |
| | GCGCCGATTGAAAAAACCATTAGCAAAGCGAAA | |
| | GGCCAGCCGCGCGAACCGCAGGTGTATACCCTGC | |
| | CGCCGAGCCGCGATGAACTGACCAAAAACCAGG | |
| | TGAGCCTGACCTGCCTGGTGAAAGGCTTTTATCC | |
| | GAGCGATATTGCGGTGGAATGGGAAAGCAACGG | |
| | CCAGCCGGAAAACAACTATAAAACCACCCCGCC | |
| | GGTGCTGGATAGCGATGGCAGCTTTTTTCTGTAT | |
| | AGCAAACTGACCGTGGATAAAAGCCGCTGGCAG | |
| | CAGGGCAACGTGTTTAGCTGCAGCGTGATGCATG | |
| | AAGCGCTGCATAACCATTATACCCAGAAAAGCCT | |
| | GAGCCTGAGCCCGGGCAAAG | |

We claim:

1. A transposon vector comprising a construct, the construct comprising:
a gene encoding a therapeutic protein operably linked to a heterologous promoter;
wherein the therapeutic protein inhibits an activator protein; and
wherein the activator protein activates transcription of the gene encoding the therapeutic protein.

2. The transposon vector of claim 1, wherein the activator protein is an inflammatory protein; and wherein the therapeutic protein is an anti-inflammatory protein.

3. The transposon vector of claim 2, wherein the promoter is selected from a C-C motif chemokine ligand 2 (CCL2) promoter, a bone morphogenetic protein responsive element (BRE) promoter, and a CAGA promoter.

4. The transposon vector of claim 2, wherein the therapeutic protein is selected from C-C motif chemokine ligand 22 (CCL22), an activin receptor type IIA-Fc fusion protein (ActRIIA-Fc), a transforming growth factor beta receptor II-Fc fusion protein (TGFβRII-Fc), and a BMPRIA-Fc fusion protein.

5. The transposon vector of claim 1, wherein the heterologous promoter is a C-C motif chemokine ligand 2 (CCL2) promoter; and wherein the therapeutic protein is C-C motif chemokine ligand 22 (CCL22).

6. The transposon vector of claim 5, wherein the construct comprises a sequence having at least 95% identity to SEQ ID NO: 1.

7. The transposon vector of claim 1, wherein the heterologous promoter is a bone morphogenetic protein responsive element (BRE) promoter; and wherein the therapeutic protein is activin receptor type IIA-Fc fusion protein (ActRIIA-Fc).

8. The transposon vector of claim 7, wherein the construct comprises a sequence having at least 95% identity to SEQ ID NO: 3.

9. The transposon vector of claim 1, wherein the heterologous promoter is a CAGA promoter; and wherein the therapeutic protein is transforming growth factor beta receptor II-Fc fusion protein (TGFβRII-Fc).

10. The transposon vector of claim 9, wherein the construct comprises a sequence having at least 95% identity to SEQ ID NO: 5.

11. The transposon vector of claim 1, wherein the heterologous promoter is a BRE promoter; and wherein the therapeutic protein is a BMPRIA-Fc fusion protein.

12. The transposon vector of claim 11, wherein the construct comprises a sequence having at least 95% identity to SEQ ID NO: 13.

13. A composition comprising the transposon vector of any one of claims 1-12; and a transposase enzyme.

14. A kit comprising the transposon vector of any one of claims 1-12; and a transposase enzyme; wherein the transposon vector and transposase enzyme are provided in separate containers.

15. The kit of claim 14, further comprising reagents for electroporation.

16. A construct comprising a sequence having at least 95% identity to SEQ ID NO: 1.

17. A construct comprising a sequence having at least 95% identity to SEQ ID NO: 3.

18. A construct comprising a sequence having at least 95% identity to SEQ ID NO: 5.

19. A construct comprising a sequence having at least 95% identity to SEQ ID NO: 13.

20. A host cell transfected with the transposon vector of any one of claims 1-12 or the construct of any one of claims 16-19; wherein the cell is capable of secreting the therapeutic protein.

21. The host cell of claim 20, wherein the host cell is a human cell or a mouse cell.

22. The host cell of claim 20, wherein the host cell is selected from an adipocyte, a bone marrow-derived cell, and a hematopoietic stem cell.

23. The host cell of claim 20, wherein the host cell is a primary cell.

24. The host cell of claim 20, wherein the host cell is an immortalized cell.

25. The host cell of claim 24, wherein the host cell is a human embryonic kidney cell.


SEQUENCE LISTING (15 sequences)

SEQ ID NO: 1 - DNA, 1423 bp, synthetic construct
  source 1..1423: mol_type = other DNA
ccgggtccgctcagccactggcacgcatgtgagagcgccactctttttatcaagagtctgctgaaatgaattctaagggctttcagattttatcgcttcgatcacactgtttgtgaagaaatctaaacctggaaagctgaattaagccagacattccagttggctcactcatggtccctggaaatcacgaaggaaacagggcagagagctaccaggagtagtaaacgctaacacacaggcttctaattcatcatggcttaaccaaaaggcatgtttctccatggcccgttagctgtctgttagtttgaaggtaatgtgatgatagccaggctggagctacctgggtcctgttccagggactcttagggcagttacctacatgacccttctggacctcagctgcctgatctgagaatggggagtaacagcatctacttaccagagctggggaaattagagactgcactgtgaagggttccaaccactcctggcccagagtaagcactaggtgagttttatatagaaatttcttctgcaccatgagctcatgtatatggctttccaggtctggacttttacaactcagacagttcatatcaatgtcacaaagcttcttagtgctgcaaaatatctggtaatcaccaagtggagagaatgctgtattcaacaaggcctgataaccaaggactcagccgacttactgggggtcctttcccagctgcaaggttccttgagccaggggcaagctaggctgtgtttccaagcatcttcttcctaaggactttaggttccttggccacttccttttatttcagtgaaagcagagccactccattcacacttgtggtcacagtagtacaattactgccaattcttccctctttccccccccccccctactccctgcgcagcttcatttgctcccaggagtggctagaaaaataccaaattccaacccacagtttctctcttccacttcctggaaacacccgagggctctgcacttactcagcggattcaacttccactttccatcacttatccagggtgatgctactccttggcaccaagcaccctgcctgactccacccccctggcttacaataaaaggctgcctcagagcagccagaagtgcacatggatatggatcgcctgcagaccgcgctgctggtggtgctggtgctgctggcggtggcgctgcaggcgaccgaagcgggcccgtatggcgcgaacatggaagatagcgtgtgctgccgcgattatgtgcgctatcgcctgccgctgcgcgtggtgaaacatttttattggaccagcgatagctgcccgcgcccgggcgtggtgctgctgacctttcgcgataaagaaatttgcgcggatccgcgcgtgccgtgggtgaaaatgattctgaacaaactgagccagt

SEQ ID NO: 2 - DNA, 8984 bp, synthetic construct
  source 1..8984: mol_type = other DNA
ctgcagaacacgcagctagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgagggacagcccccccccaaagcccccagggatgtaattacgtccctcccccgctagtgggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccagactagtaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggcggccgctcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagctagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggtaccccgggtccgctcagccactggcacgcatgtgagagcgccactctttttatcaagagtctgctgaaatgaattctaagggctttcagattttatcgcttcgatcacactgtttgtgaagaaatctaaacctggaaagctgaattaagccagacattccagttggctcactcatggtccctggaaatcacgaaggaaacagggcagagagctaccaggagtagtaaacgctaacacacaggcttctaattcatcatggcttaaccaaaaggcatgtttctccatggcccgttagctgtctgttagtttgaaggtaatgtgatgatagccaggctggagctacctgggtcctgttccagggactcttagggcagttacctacatgacccttctggacctcagctgcctgatctgagaatggggagtaacagcatctacttaccagagctggggaaattagagactgcactgtgaagggttccaaccactcctggcccagagtaagcactaggtgagttttatatagaaatttcttctgcaccatgagctcatgtatatggctttccaggtctggacttttacaactcagacagttcatatcaatgtcacaaagcttcttagtgctgcaaaatatctggtaatcaccaagtggagagaatgctgtattcaacaaggcctgataaccaaggactcagccgacttactgggggtcctttcccagctgcaaggttccttgagccaggggcaagctaggctgtgtttccaagcatcttcttcctaaggactttaggttccttggccacttccttttatttcagtgaaagcagagccactccattcacacttgtggtcacagtagtacaattactgccaattcttccctctttccccccccccccctactccctgcgcagcttcatttgctcccaggagtggctagaaaaataccaaattccaacccacagtttctctcttccacttcctggaaacacccgagggctctgcacttactcagcggattcaacttccactttccatcacttatccagggtgatgctactccttggcaccaagcaccctgcctgactccacccccctggcttacaataaaaggctgcctcagagcagccagaagtgcacatggatatggatcgcctgcagaccgcgctgctggtggtgctggtgctgctggcggtggcgctgcaggcgaccgaagcgggcccgtatggcgcgaacatggaagatagcgtgtgctgccgcgattatgtgcgctatcgcctgccgctgcgcgtggtgaaacatttttattggaccagcgatagctgcccgcgcccgggcgtggtgctgctgacctttcgcgataaagaaatttgcgcggatccgcgcgtgccgtgggtgaaaatgattctgaacaaactgagccagtaagaattcgtcgactgtacaacgcgtgtttaaacttcgaatctagatacgtacatatgatttaaatgatctaactaactaaacttgtttattgcacttataatggttacaaataaagcaatagcatcacaaatttcacaaataaagcatttttttcactgcattctagttgtggtttgtccaaactcatcaatgtatcttatcatgtctggaatgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtgtaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagtta

SEQ ID NO: 3 - DNA, 1316 bp, synthetic construct
  source 1..1316: mol_type = other DNA
ttaattaagctagctcagaccgttagacgccaggacgggctgtcaggctggcgccgcggcgccagcctgacagcccgtcctggcgtctaacggtctgagctagcccgggctcgagatctgggctataaaagggggtgggggcgcgttcgtcctcactctcttccaagcttggcattccggtactgttggtaaagccaccatgggcgcggcggcgaaactggcgtttgcggtgtttctgattagctgcagcagcggcgcgattctgggccgcagcgaaacccaggaatgcctgttttttaacgcgaactgggaaaaagatcgcaccaaccagaccggcgtggaaccgtgctatggcgataaagataaacgccgccattgctttgcgacctggaaaaacattagcggcagcattgaaattgtgaaacagggctgctggctggatgatattaactgctatgatcgcaccgattgcgtggaaaaaaaagatagcccggaagtgtatttttgctgctgcgaaggcaacatgtgcaacgaaaaatttagctattttccggaaatggaagtgacccagccgaccagcaacccggtgaccccgaaaccgccgattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaag

SEQ ID NO: 4 - DNA, 9623 bp, synthetic construct
  source 1..9623: mol_type = other DNA
cagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccttgcgtttaaacagatctacgcgtttaattaagctagctcagaccgttagacgccaggacgggctgtcaggctggcgccgcggcgccagcctgacagcccgtcctggcgtctaacggtctgagctagcccgggctcgagatctgggctataaaagggggtgggggcgcgttcgtcctcactctcttccaagcttggcattccggtactgttggtaaagccaccatgggcgcggcggcgaaactggcgtttgcggtgtttctgattagctgcagcagcggcgcgattctgggccgcagcgaaacccaggaatgcctgttttttaacgcgaactgggaaaaagatcgcaccaaccagaccggcgtggaaccgtgctatggcgataaagataaacgccgccattgctttgcgacctggaaaaacattagcggcagcattgaaattgtgaaacagggctgctggctggatgatattaactgctatgatcgcaccgattgcgtggaaaaaaaagatagcccggaagtgtatttttgctgctgcgaaggcaacatgtgcaacgaaaaatttagctattttccggaaatggaagtgacccagccgaccagcaacccggtgaccccgaaaccgccgattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctactaacttcagcctgctgaagcaggctggcgacgtggaggagaaccctggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaatttaaatcggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgcggccgcaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaa

SEQ ID NO: 5 - DNA, 1495 bp, synthetic construct
  source 1..1495: mol_type = other DNA
cccgggctcgagagccagacaaaaagccagacatttagccagacactcgagagccagacaaaaagccagacatttagccagacactcgagagccagacaaaaagccagacatttagccagacactcgagtgtctggctaaatgtctggctttttgtctggctctcgagatctgggctataaaagggggtgggggcgcgttcgtcctcactctcttccaagcttaggcgtgtacggtgggaggcctatataagcagagctcgtttagtgaaccgtcagatcgcactagtccaagtttgtacaaaaaagcaggctgccaccatgggccgcggcctgctgcgcggcctgtggccgctgcatattgtgctgtggacccgcattgcgagcaccattccgccgcatgtgcagaaaagcgtgaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggatatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 6 - DNA, 7929 bp, synthetic construct
  source 1..7929: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgcccgggctcgagagccagacaaaaagccagacatttagccagacactcgagagccagacaaaaagccagacatttagccagacactcgagagccagacaaaaagccagacatttagccagacactcgagtgtctggctaaatgtctggctttttgtctggctctcgagatctgggctataaaagggggtgggggcgcgttcgtcctcactctcttccaagcttaggcgtgtacggtgggaggcctatataagcagagctcgtttagtgaaccgtcagatcgcactagtccaagtttgtacaaaaaagcaggctgccaccatgggccgcggcctgctgcgcggcctgtggccgctgcatattgtgctgtggacccgcattgcgagcaccattccgccgcatgtgcagaaaagcgtgaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggatatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaaggaagcggagccacgaacttctctctgttaaagcaagcaggagatgttgaagaaaaccccgggcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 7 - DNA, 1137 bp, synthetic construct
  source 1..1137: mol_type = other DNA
ccgggtccgctcagccactggcacgcatgtgagagcgccactctttttatcaagagtctgctgaaatgaattctaagggctttcagattttatcgcttcgatcacactgtttgtgaagaaatctaaacctggaaagctgaattaagccagacattccagttggctcactcatggtccctggaaatcacgaaggaaacagggcagagagctaccaggagtagtaaacgctaacacacaggcttctaattcatcatggcttaaccaaaaggcatgtttctccatggcccgttagctgtctgttagtttgaaggtaatgtgatgatagccaggctggagctacctgggtcctgttccagggactcttagggcagttacctacatgacccttctggacctcagctgcctgatctgagaatggggagtaacagcatctacttaccagagctggggaaattagagactgcactgtgaagggttccaaccactcctggcccagagtaagcactaggtgagttttatatagaaatttcttctgcaccatgagctcatgtatatggctttccaggtctggacttttacaactcagacagttcatatcaatgtcacaaagcttcttagtgctgcaaaatatctggtaatcaccaagtggagagaatgctgtattcaacaaggcctgataaccaaggactcagccgacttactgggggtcctttcccagctgcaaggttccttgagccaggggcaagctaggctgtgtttccaagcatcttcttcctaaggactttaggttccttggccacttccttttatttcagtgaaagcagagccactccattcacacttgtggtcacagtagtacaattactgccaattcttccctctttccccccccccccctactccctgcgcagcttcatttgctcccaggagtggctagaaaaataccaaattccaacccacagtttctctcttccacttcctggaaacacccgagggctctgcacttactcagcggattcaacttccactttccatcacttatccagggtgatgctactccttggcaccaagcaccctgcctgactccacccccctggcttacaataaaaggctgcctcagagcagccagaagtgcac

SEQ ID NO: 8 - DNA, 280 bp, synthetic construct
  source 1..280: mol_type = other DNA
atggatcgcctgcagaccgcgctgctggtggtgctggtgctgctggcggtggcgctgcaggcgaccgaagcgggcccgtatggcgcgaacatggaagatagcgtgtgctgccgcgattatgtgcgctatcgcctgccgctgcgcgtggtgaaacatttttattggaccagcgatagctgcccgcgcccgggcgtggtgctgctgacctttcgcgataaagaaatttgcgcggatccgcgcgtgccgtgggtgaaaatgattctgaacaaactgagccagt

SEQ ID NO: 9 - DNA, 199 bp, synthetic construct
  source 1..199: mol_type = other DNA
ttaattaagctagctcagaccgttagacgccaggacgggctgtcaggctggcgccgcggcgccagcctgacagcccgtcctggcgtctaacggtctgagctagcccgggctcgagatctgggctataaaagggggtgggggcgcgttcgtcctcactctcttccaagcttggcattccggtactgttggtaaagccacc

SEQ ID NO: 10 - DNA, 1117 bp, synthetic construct
  source 1..1117: mol_type = other DNA
atgggcgcggcggcgaaactggcgtttgcggtgtttctgattagctgcagcagcggcgcgattctgggccgcagcgaaacccaggaatgcctgttttttaacgcgaactgggaaaaagatcgcaccaaccagaccggcgtggaaccgtgctatggcgataaagataaacgccgccattgctttgcgacctggaaaaacattagcggcagcattgaaattgtgaaacagggctgctggctggatgatattaactgctatgatcgcaccgattgcgtggaaaaaaaagatagcccggaagtgtatttttgctgctgcgaaggcaacatgtgcaacgaaaaatttagctattttccggaaatggaagtgacccagccgaccagcaacccggtgaccccgaaaccgccgattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaag

SEQ ID NO: 11 - DNA, 290 bp, synthetic construct
  source 1..290: mol_type = other DNA
cccgggctcgagagccagacaaaaagccagacatttagccagacactcgagagccagacaaaaagccagacatttagccagacactcgagagccagacaaaaagccagacatttagccagacactcgagtgtctggctaaatgtctggctttttgtctggctctcgagatctgggctataaaagggggtgggggcgcgttcgtcctcactctcttccaagcttaggcgtgtacggtgggaggcctatataagcagagctcgtttagtgaaccgtcagatcgcactagtcc

SEQ ID NO: 12 - DNA, 1176 bp, synthetic construct
  source 1..1176: mol_type = other DNA
atgggccgcggcctgctgcgcggcctgtggccgctgcatattgtgctgtggacccgcattgcgagcaccattccgccgcatgtgcagaaaagcgtgaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggatatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 13 - DNA, 1367 bp, synthetic construct
  source 1..1367: mol_type = other DNA
ttaattaagctagctcagaccgttagacgccaggacgggctgtcaggctggcgccgcggcgccagcctgacagcccgtcctggcgtctaacggtctgagctagcccgggctcgagatctgggctataaaagggggtgggggcgcgttcgtcctcactctcttccaagcttggcattccggtactgttggtaaagccaccatgccgcagctgtatatttatattcgcctgctgggcgcgtatctgtttattattagccgcgtgggcggccagaacctggatagcatgctgcatggcaccggcatgaaaagcgatagcgatcagaaaaaaagcgaaaacggcgtgaccctggcgccggaagataccctgccgtttctgaaatgctattgcagcggccattgcccggatgatgcgattaacaacacctgcattaccaacggccattgctttgcgattattgaagaagatgatcagggcgaaaccaccctggcgagcggctgcatgaaatatgaaggcagcgattttcagtgcaaagatagcccgaaagcgcagctgcgccgcaccattgaatgctgccgcaccaacctgtgcaaccagtatctgcagccgaccctgccgccggtggtgattggcccgttttttgatggcagcattcgcattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaag

SEQ ID NO: 14 - DNA, 9674 bp, synthetic construct
  source 1..9674: mol_type = other DNA
cagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccttgcgtttaaacagatctacgcgtttaattaagctagctcagaccgttagacgccaggacgggctgtcaggctggcgccgcggcgccagcctgacagcccgtcctggcgtctaacggtctgagctagcccgggctcgagatctgggctataaaagggggtgggggcgcgttcgtcctcactctcttccaagcttggcattccggtactgttggtaaagccaccatgccgcagctgtatatttatattcgcctgctgggcgcgtatctgtttattattagccgcgtgggcggccagaacctggatagcatgctgcatggcaccggcatgaaaagcgatagcgatcagaaaaaaagcgaaaacggcgtgaccctggcgccggaagataccctgccgtttctgaaatgctattgcagcggccattgcccggatgatgcgattaacaacacctgcattaccaacggccattgctttgcgattattgaagaagatgatcagggcgaaaccaccctggcgagcggctgcatgaaatatgaaggcagcgattttcagtgcaaagatagcccgaaagcgcagctgcgccgcaccattgaatgctgccgcaccaacctgtgcaaccagtatctgcagccgaccctgccgccggtggtgattggcccgttttttgatggcagcattcgcattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctactaacttcagcctgctgaagcaggctggcgacgtggaggagaaccctggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaatttaaatcggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgcggccgcaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtcgacaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaa

SEQ ID NO: 15 - DNA, 1168 bp, synthetic construct
  source 1..1168: mol_type = other DNA
atgccgcagctgtatatttatattcgcctgctgggcgcgtatctgtttattattagccgcgtgggcggccagaacctggatagcatgctgcatggcaccggcatgaaaagcgatagcgatcagaaaaaaagcgaaaacggcgtgaccctggcgccggaagataccctgccgtttctgaaatgctattgcagcggccattgcccggatgatgcgattaacaacacctgcattaccaacggccattgctttgcgattattgaagaagatgatcagggcgaaaccaccctggcgagcggctgcatgaaatatgaaggcagcgattttcagtgcaaagatagcccgaaagcgcagctgcgccgcaccattgaatgctgccgcaccaacctgtgcaaccagtatctgcagccgaccctgccgccggtggtgattggcccgttttttgatggcagcattcgcattgaaggccgcatggatccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaag